(19) United States
(12) Patent Application Publication
(10) Pub. No.: US 2016/0237074 A1
(43) Pub. Date: Aug. 18, 2016

(54) COMPOUNDS, COMPOSITIONS AND METHODS FOR THE TREATMENT OF DEGENERATIVE DISEASES AND DISORDERS

(71) Applicant: MUSC FOUNDATION FOR RESEARCH DEVELOPMENT, Charleston, SC (US)

(72) Inventors: Craig C. BEESON, Charleston, SC (US); Baerbel ROHRER, Charleston, SC (US); Nathan Perron, Charleston, SC (US)

(73) Assignee: MUSC FOUNDATION FOR RESEARCH DEVELOPMENT (10 pp.)

(21) Appl. No.: 14/996,662

(22) Filed: Jan. 15, 2016

Related U.S. Application Data
(63) Continuation of application No. 13/636,754, filed on Feb. 7, 2013, now abandoned.
(60) Provisional application No. 61/317,185, filed on Mar. 24, 2010.

(51) Int. Cl. C07D 417/04

(52) U.S. Cl. CPC C07D 417/04

(57) ABSTRACT

Disclosed are compounds or pharmaceutically acceptable salts thereof, having the structure of formula I. Also disclosed are methods of preventing and/or treating degenerative disease in a subject, comprising administering to said subject a therapeutically effective amount of a compound of formula I, or a pharmaceutically acceptable salt thereof. Also disclosed are pharmaceutical compositions for preventing and/or treating degenerative disease in a subject comprising a therapeutically effective amount of a compound of formula I, or a pharmaceutically acceptable salt thereof.

### I. BACKGROUND

Degenerative diseases are diseases in which the function or structure of the affected tissues or organs will progressively deteriorate over time. Some examples of degenerative diseases are retinal degenerative disease, e.g., age-related mascular degeneration, Stargardt's disease, glaucoma, retinitis pigmnentosa, and optic nerve degeneration; Amyotrophic Lateral Sclerosis (ALS), e.g., Lou Gehrig's Disease; Alzheimer's disease; Parkinson's Disease; Multiple system atrophy; Niemann Pick disease; Atherosclerosis; Progressive supranuclear palsy; Cancer; Tay-Sachs Disease; Diabetes; Heart Disease; Keratoconus; Inflammatory Bowel Disease (IBD); Prostatitis; Osteoarthritis; Osteoporosis; Rheumatoid Arthritis; and Huntingtons Disease. It has been known that mitochondrial damage and/or dysfunction causes degenerative diseases.

Mitochondria are cellular organelles present in most eukaryotic cells. One of their primary functions is oxidative phosphorylation, a process through which energy derived from metabolism of fuels like glucose or fatty acids is converted to ATP, which is then used to drive various energy-requiring biosynthetic reactions and other metabolic activities. Mitochondria have their own genomes, separate from nuclear DNA, comprising rings of DNA with about 16,000 base pairs in human cells. Each mitochondrion may have multiple copies of its genome, and individual cells may have hundreds of mitochondria. In addition to supplying cellular energy, mitochondria are involved in a range of other processes, such as signaling, cellular differentiation, cell death, as well as the control of the cell cycle and cell growth (McBride et al., Curr. Biol., 2006, 16 (14): R551).

As mitochondria produce ATP, they simultaneously yield reactive oxygen species (ROS), which are harmful free radicals that circulate throughout the cell, the mitochondria, and the body, causing more damage. The circulation of ROS leads to the activation of reactive nitrogen compounds, which in turn induce, or activate, genes in the DNA that are associated with many degenerative diseases. The DNA for each mitochondrion (mtDNA) remains unprotected within the membrane of the mitochondrion itself. In comparison to the DNA in the nucleus of the cell (nDNA), mtDNA is easily damaged by free radicals and the ROS that it produces. Freely floating mtDNA lacks protective measures associated with nDNA, and therefore suffers from multiple mutations. It has been estimated that the lack of protective measures results in mutations to mtDNA occurring 10 to 20 times more frequently than mutations to nDNA.

Mitochondrial damage and/or dysfunction contribute to various disease states. Some diseases are due to mutations or deletions in the mitochondrial genome. Mitochondria divide and proliferate with a faster turnover rate than their host cells, and their replication is under control of the nuclear genome. If a threshold proportion of mitochondria in a cell is defective, and if a threshold proportion of such cells within a tissue have defective mitochondria, symptoms of tissue or organ dysfunction can result. Practically any tissue can be affected, and a large variety of symptoms can be present, depending on the extent to which different tissues are involved.

A fertilized ovum might contain both normal and genetically defective mitochondria. The segregation of defective mitochondria into different tissues during division of this ovum is a stochastic process, as will be the ratio of defective to normal mitochondria within a given tissue or cell (although there can be positive or negative selection for defective mitochondrial genomes during mitochondrial turnover within cells). Thus, a variety of different pathologic phenotypes can emerge out of a particular point mutation in mitochondrial DNA. Conversely, similar phenotypes can emerge from mutations or deletions affecting different genes within mitochondrial DNA. Clinical symptoms in congenital mitochondrial diseases often manifest in postmitotic tissues with high energy demands like brain, muscle, optic nerve, and myocardium, but other tissues including endocrine glands, liver, gastrointestinal tract, kidney, and hematopoietic tissue are also involved, again depending in part on the segregation of mitochondria during development, and on the dynamics of mitochondrial turnover over time.

In addition to congenital disorders involving inherited defective mitochondria, acquired mitochondrial damage and/or dysfunction contribute to diseases, particularly neurodegenerative disorders associated with aging like Parkinson's, Alzheimer's, Huntington's Diseases. The incidence of somatic mutations in mitochondrial DNA rises exponentially with age; and diminished respiratory chain activity is found universally in aging people. Mitochondrial dysfunction is also implicated in excitotoxic neuronal injury, such as that associated with seizures or ischemia.

Other pathologies with etiology involving mitochondrial damage and/or dysfunction include schizophrenia, bipolar disorder, dementia, epilepsy, stroke, cardiovascular disease, retinal degenerative disease (e.g., age-related mascular degeneration, Stargardt's disease, glaucoma, retinitis pigmnentosa, and optic nerve degeneration), and diabetes mellitus. A common thread thought to link these seemingly-unrelated conditions is cellular damage causing oxidative stress. Oxidative stress is caused by an imbalance between the production of reactive oxygen and a biological system's ability to readily detoxify the reactive intermediates or easily repair the resulting damage. All forms of life maintain a reducing environment within their cells. This reducing environment is preserved by enzymes that maintain the reduced state through a constant input of metabolic energy. Disturbances in this normal redox state can cause toxic effects through the production of peroxides and free radicals that damage all components of the cell, including proteins, lipids, and DNA.

Treatment of degenerative diseases involving mitochondrial damage and/or dysfunction has heretofore involved administration of vitamins and cofactors used by particular elements of the mitochondrial respiratory chain. Coenzyme Q (ubiquinone), nicotinamide, riboflavin, carnitine, biotin, and lipoic acid are used in patients with occasional benefit, especially in disorders directly stemming from primary deficiencies of one of these cofactors. However, while useful in isolated cases, no such metabolic cofactors or vitamins have been shown to have general utility in clinical practice in treating degenerative diseases involving mitochondrial damage and/or dysfunction. Therefore, there is a need existing for new drug therapies for the treatment of subjects suffering from or susceptible to the above disorders or conditions associated with mitochondrial damage and/or dysfunction. In particular, a need still exists for new drugs having one or more improved properties (such as safety profile, efficacy, or physical properties) relative to those currently available.

### II. SUMMARY

Disclosed are compositions and methods for the prevention and treatment of degenerative diseases. For example, disclosed are a class of compounds, including the pharmaceutically acceptable salts of the compounds, having the structure of formula I:

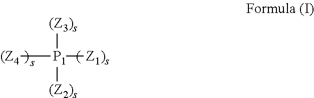

wherein:

P₁ is a pharmacophore where the structure of said pharmacophore comprising one or more hydrogen bond donor and/or acceptor groups;

Z₁ is (L₁)_{s′}-P₂;

Z₂ is -(L₂)_{s′}-P₃-(L₃)_{s′}-P₄;

Z₃ is -(L₄)_{s′}-P₅;

Z₄ is -(L₅)_{s′}-P₆;

s and s′ are each independently a subscript selected from 0 to 5;

P₂ is a pharmacophore where the structure of said pharmacophore comprising one or more hydrogen bond donor and/or acceptor groups; and/or one or more hydrophobic groups;

P₃ is a pharmacophore where the structure of said pharmacophore comprising one or more hydrogen bond donor and/or acceptor groups;

P₄ is a pharmacophore where the structure of said pharmacophore comprising one or more hydrophobic groups;

P₅ is a pharmacophore where the structure of said pharmacophore comprising one or more hydrophobic groups;

P₆ is a pharmacophore where the structure of said pharmacophore comprising one or more hydrophobic groups;

L₁, L₂, L₃, L₄ and L₅ are each independently a linker selected from the group consisting of —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰² wherein each of said group is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰²;

R¹⁰¹ and R¹⁰² are each independently selected from the group consisting of hydrogen, alkyl, alkenyl, alkynyl, cycloalkyl, aryl, heterocycloalkyl and heteroaryl; wherein each R¹⁰¹ and R¹⁰² alkyl, alkenyl, alkynyl, cycloalkyl, aryl, heterocycloalkyl or heteroaryl is optionally independently substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl;

wherein said one or more hydrogen bond donor and/or acceptor groups, and said one or more hydrophobic groups are each optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰² wherein each of said substitutent is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰²;

wherein each of L₁, L₂, L₃, L₄ and L₅ is optionally fused with the adjacent one or more pharmacophores.

Also disclosed are methods of preventing and/or treating degenerative disease in a subject, comprising administering to said subject a therapeutically effective amount of a compound of formula I, or a pharmaceutically acceptable salt thereof. Also disclosed are pharmaceutical compositions for preventing and/or treating degenerative disease in a subject comprising a therapeutically effective amount of a compound of formula I, or a pharmaceutically acceptable salt thereof.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows an in vitro efficacy study of compounds CB11, CB11a, CB11b, CB11c, CB11d and IBMX against the calcium-induced mitochondrial damage assay.

FIG. 2 shows an in vitro efficacy study of compounds CB12, CB12a, CB12b, CB12c, CB12d and IBMX against the calcium-induced mitochondrial damage assay.

FIG. 3 shows an in vitro efficacy study of compound CB11 against rd1 Mouse retinal organ culture assay (image result).

FIG. 4 shows an in vitro efficacy study of compound CB11 against rd1 Mouse retinal organ culture assay (quantitative result from the image).

FIG. 5 shows an in vitro efficacy study of compound CB12 against rd1 Mouse retinal organ culture assay (image result).

FIG. 6 shows an in vitro efficacy study of compound CB12 against rd1 Mouse retinal organ culture assay (quantitative result from the image).

FIG. 7 shows an in vivo efficacy study of compound CB11 against light damage by administering daily eyedrops of CB11 solution (10 μL of 1 mM stock) during 10 days light damage (continuous exposure) in mice.

FIG. 8 shows an in vivo efficacy study of compound CB11 against light damage by administering daily eyedrops of CB11 solution (10 μL of 1 mM stock) during 10 days light damage (continuous exposure) in mice (Electroretinography).

FIG. 9 shows an in vivo efficacy study of compound CB11 against light damage by administering daily eyedrops of CB11 solution (10 μL of 1 mM stock) during 10 days light damage (continuous exposure) in mice (quantitative result from Electroretinography).

FIG. 10 shows an overlap of liphophilic and electronegative properties between compounds CB11 and CB12.

FIG. 11 shows a spatial overlap of physicochemical features such as hydrophobicity, hydrogen bond donor/acceptors, polar regions between CB11 and CB12.

FIG. 12 shows a seven point consensus pharmacophore between CB11 and CB12.

FIG. 13 shows a spatial connection and arrangement of the seven point consensus pharmacophores between CB11 and CB12.

FIG. 14. Pharmacophore overlap analyses of structural variants for molecules of formula II. The upper left shows a thiazole that maps to the pharmacophore and was tested. The other five successive images show how different structural variants of thiazoles, oxazoles, and pyrazoles overlap with the pharmacophore-overlap is designated with meshed spheres.

FIG. 15. Pharmacophore overlap analyses of structural variants for molecules of formulae II. The images show how different structural variants of thiazoles, oxazoles, and pyrazoles overlap with the pharmacophore-overlap is designated with meshed spheres. Shown also is how the MOE software can rank goodness of ft based on both #-dimensional overlap and path lengths between functional groups that led to the definition of formula I.

FIG. 16 shows a seven point consensus pharmacophore between CB11, CB12, CB12_1 and CB11_3.

FIG. 17. Calcium and oxidative stress attenuate mitochondrial capacity. Shown are 661W cell OCR responses to 1 μM FCCP after treatment with listed concentrations of (A) A23187 and (B) tBuOOH. In A OCR rates are given as pmol/min, in B the rates for each well have been normalized to the basal rates measured prior to FCCP (which standardizes comparisons between data measured on different days). 661W cells analyzed by XF24 were incubated for 24 h and viability determined using ethidium bromide/acridine orange. Images of cells treated with (C) 1 M A23187 and (D) control represent 47% and >95% viability.

FIG. 18. Calcium and oxidative stress produce metabolic phenotypes in 661W cells that are correlated with cell death. Shown in panels A and B are multivariate analyses of ECAR vs OCR with viability represented by color. The concentrations of A23187 and tBuOOH increase from right to left and the metabolic rates are plotted as percent change from vehicle control cells. 6-PFK mRNA levels, surrogate measures for ECAR in retinas, are plotted against time for rd1(C) and light damaged retinas (D) Please refer to Appendix 3 for more information [16].

FIG. 19. Mild calcium or oxidative stress reduces metabolic capacity in a photoreceptor cell line. (Calcium stress, left panel) 661W cells were treated with vehicle control (dark blue) or 1.0 mM IBMX (turquoise), followed by treatment with 1 μM FCCP. The direct effects of IBMX upon respiratory capacity can easily be observed using the respiratory uncoupler FCCP, which shows that mitochondria treated with 1.0 mM IBMX have a diminished respiratory capacity compared to control. (Oxidant stress, right panel)

FIG. 20. Images of 661W treated with the cellular stressors A23187, IBMX, paraquat (Pq), and tBuOOH at various concentrations. Cells were stained for 30 min with Hoechst 33342 (blue) and propidium iodide (red), to give a relative analysis of live cells (blue) vs. dead cells (red) as a function of stressor concentration. Images were taken on a GE Healthcare IN-Cell 1000 using black-walled 96-well plates with optically clear TC surface. The cells were plated at a density of 20,000/well and grown in DMEM+5% FBS for 48 h before treating with stressors for 24 h.

FIG. 21. Secondary screening of neuroprotective agents discovered from library screening. The 661W cells were pretreated for 1 h with lead compounds, CB10-CB12 (1 μM), prior to treatment with 600 μM IBMX. The basal and uncoupled OCR rates were measured. Note that IBMX treatment attenuates basal and uncoupled OCR relative to untreated control and that the agents CB11 and CB12 protect against this loss in respiratory capacity.

FIG. 22. Chemical structures of CB11 and CB12, and their corresponding generic structures.

FIG. 23. Chemotype discrimination from HTS leads. Two lead molecules, CB11 and CB12, were found to overlap in chemical space to define a pharmacophore with nearly 100% overlap for seven physicochemical parameters. Shown in A are the two molecules overlapped where the spheres identify regions of physicochemical overlap. Note that the central regions of the molecules are discordant because they are two distinct structural classes. Shown in B is the 3D pharmacophore that defines the chemotype for the cytoprotective agent.

FIG. 24. These are frozen sections of rd1 retina-RPE sandwich cultures grown in culture from post-natal day (P)10 through P21. Compounds were replaced with media changes (every 48 hrs). The left-hand image is a vehicle-treated control retina from an rd1 mouse; the middle image is of an rd1 retina treated with calpeptin, which blocks calpain, preventing apoptotic cell death (positive control). The right-hand image of an rd1 retina treated with CB11, which was found to protect rd1 photoreceptors comparable to calpeptin.

FIG. 25. These are frozen sections of rd1 retina-RPE sandwich cultures grown in culture from post-natal day (P)10 through P21. Compounds were replaced with media changes (every 48 hrs). The left-hand image is a vehicle-treated control retina from an rd1 mouse; the middle image is of an rd1 retina treated with calpeptin, which blocks calpain, preventing apoptotic cell death (positive control). The right-hand image of an rd1 retina treated with CB11, which was found to protect rd1 photoreceptors comparable to calpeptin. The right panel quantifies and summarizes the images from the left slide. An untreated rd1 retina has about 1 row of photoreceptors remaining at P21; calpeptin-treated retinas have about 3.5 rows, CB11-treated retinas also have about 3.5 rows, and wild-type control retinas have almost 7 rows.

FIG. 26. CB11 was formulated into an aqueous solution containing 1 mM CB11 dissolved in 2% ethanol, 0.5% Brij-78, and 0.9% NaCl in water. The animal model used was the constant light model in Balb/c mice, in which the rod photoreceptors die of oxidative stress, resulting in ˜50% cell loss within 10 days. Animals were treated with CB11 by administering 10 μL eyedrops at the time-points indicated in the graph (1 drop in the PM, or one eyedrop every 12 hours) over the 10 days of continuous light. After 10 days, the mice were sacrificed and the rows of photoreceptors were manually counted as in the previous slides.

FIG. 27. Electroretinography (ERG) is a tool to measure the response of the entire retina to a flash of light, using corneal surface electrodes. The negative deflection is the response of the photoreceptors, while the positive deflection is a response of the first set of interneurons, the rod bipolar cells (and hence tests synaptic transmission). Each animal was tested prior to light damage (baseline, red trace) and after light damage (black trace). Two examples are shown of mice after 10 days of eyedrops containing either saline (control) or CB11 formulation (CB11). Control animals had significantly smaller ERG amplitudes compared to those receiving daily CB11 treatment, demonstrating that mice receiving CB11 had significantly better retinal function (i.e., could see better) after 10 days of continuous light damage than vehicle treated mice. This graph quantifies the percent of the baseline ERG amplitude by retinas of sacrificed mice in response to different intensities of light. The ERG response at each light intensity was significantly improved by CB11.

### IV. DETAILED DESCRIPTION

Before the present compounds, compositions, articles, devices, and/or methods are disclosed and described, it is to be understood that they are not limited to specific synthetic methods or specific treatment methods unless otherwise specified, or to particular reagents unless otherwise specified, as such may, of course, vary. It is also to be understood that the terminology used herein is for the purpose of describing particular embodiments only and is not intended to be limiting.

### A. EYES, MITOCHONDRIA, DISEASES, AND ASSAYS

Retinal degeneration can be triggered by a number of different underlying causes; including environmental insults as well as genetic mutations (see Retnet.org for a summary on retinal disease genes). Currently, 191 loci for human retinal degeneration have been identified, 140 of these loci have genes associated with them. However, for most disease genes, their role in disease pathology is unclear. Also, disease processes appears to be influenced by a number of environmental insults making it difficult to identify one central cause. Indeed, the genetics of macular degeneration and other forms of retinal degeneration point to multiple targets and causes, each of which may be involved in a subset of patients, ultimately leading to the common endpoint of failed central vision. As current therapies for AMD are limited and successful therapies for RP have yet to be identified, there remains a significant unmet need for therapeutic approaches to treating retinal degeneration.

The neurons of the retina require large amounts of ATP (adenosine triphosphate); ATP is the universal energy currency of all known living organisms. The majority of this ATP is produced in cellular organelles called mitochondria. ATP requires simple and complex sugars or lipids as an energy source. Mitochondrial function and hence ATP production are very sensitive to environmental challenges and aging; tissues from elderly patients show a general decrease in ATP production. Photoreceptor cell lines have been used with an instrument to show that toxic agents cause significant changes in ATP metabolism within 30 min after application even though cell death is not evident until at least 24-48 h. Thus, disclosed herein changes in energy metabolism can be a major factor in disease pathogenesis and that preservation of the metabolism can provide therapeutic approaches.

Pharmacological agents to treat retinal degeneration were identified by screening a library consisting of more than 50,000 compounds from the DIVERSet collection from ChemBridge. This is a unique, collection of synthetic small molecules, forming a library that covers the maximum pharmacophore diversity with the minimum number of compounds. Four successive rounds of screening, increasing in complexity, were performed. As a primary screen, survival assays in a photoreceptor cell line were used, identifying compounds that protected against toxic insults (high calcium; oxidative stress). Second, the Seahorse Biosciences instrument to non-invasively examine mitochondrial function in vitro was used to screen. Third, the potential neuroprotectants were tested on intact retinas to determine whether they can improve photoreceptor viability in diseased retinas. Finally, the compounds were tested in vivo in a murine model for photoreceptor cell stress. The results of these studies provided the compounds and compositions disclosed herein, which are protective for mitochondria and which can be used to slow down or prevent photoreceptor degeneration.

1. Photoreceptor Degeneration and Energy Metabolism

Photoreceptor cells have unusually high metabolic demands due to the high ATP cost for converting light to a neurochemical signal [Stone J, Maslim J, Valter-Kocsi K, Mervin K, Bowers F, Chu Y, Barnett N, Provis J, Lewis G, Fisher S K et al: Mechanisms of photoreceptor death and survival in mammalian retina. Prog Retin Eye Res 1999, 18(6):689-735]. Thus, changes that alter energy metabolism or oxygen tension in the outer retina photoreceptor can result in degeneration [Pierce E A: Pathways to photoreceptor cell death in inherited retinal degenerations. Bioessays 2001, 23(7):605-618.]. Numerous studies suggest that apoptosis is the mechanism of cell death in human photoreceptor degeneration and in their respective genetic mouse models [Travis G H: Mechanisms of cell death in the inherited retinal degenerations. Am J Hum Genet 1998, 62(3):503-508.], which suggests that the “point of no return” is the mitochondrial membrane permeabilization transition [Mattson M P, Kroemer G: Mitochondria in cell death: novel targets for neuroprotection and cardioprotection. Trends Mol Med 2003, 9(5):196-205.]. It is thus perhaps not surprising to find that defects in mitochondrial pathways that produce ATP underlie a number of retinal pathologies. For example, in the rd1 mouse model for photoreceptor degeneration in retinitis pigmentosa (RP), some of the earliest damage signs are alterations in lactate dehydrogenase and Na⁺/K⁺ ATPase activities [Acosta M L, Fletcher E L, Azizoglu S, Foster L E, Farber D B, Kalloniatis M: Early markers of retinal degeneration in rd/rd mice. Mol Vis 2005, 11:717-728.]). In the RCS rat, Graymore [Graymore C: Metabolism of the Developing Retina. 7. Lactic Dehydrogenase Isoenzyme in the Normal and Degenerating Retina. a Preliminary Communication. Exp Eye Res 1964, 89:5-8] found alterations in retinal energy metabolism that precede degeneration and Vingolo et al. have improved the maximum electroretinogram responses of RP patients with hyperbaric oxygen treatments [Vingolo E M, De Mattia G, Giusti C, Forte R, Laurenti O, Pannarale M R: Treatment of nonproliferative diabetic retinopathy with Defibrotide in noninsulin-dependent diabetes mellitus: a pilot study. Acta Ophthalmol Scand 1999, 77(3):315-320]. Finally, Pierce and co-workers [Pierce E A, Quinn T, Meehan T, McGee T L, Berson E L, Dryja T P: Mutations in a gene encoding a new oxygen-regulated photoreceptor protein cause dominant retinitis pigmentosa. Nat Genet 1999, 22(3):248-254] identified a gene that encodes a new oxygen-regulated photoreceptor protein that causes dominant RP when mutated. Thus, disclosed herein early changes in energy metabolism underlie a number of retinal pathologies. Agents that ameliorate the dysregulation of energy metabolism are disclosed herein and can be developed into therapeutic strategies for treatment of retinal damage, as well as mitochondrial degeneration diseases.

2. Models of Photoreceptor Degeneration

In previous publications it has shown that 661W cells can be utilized [Tan E, Ding X Q, Saadi A, Agarwal N, Naash M I, Al-Ubaidi M R: Expression of cone-photoreceptor-specific antigens in a cell line derived from retinal tumors in transgenic mice. Invest Ophthalmol Vis Sci 2004, 45(3):764-768] treated with the Ca²⁺-ionophore A23187, cGMP gated channel agonist 8-Bromo-cGMP, or phosphodiesterase inhibitor IBMX, to mimic the increased Ca²⁺ influx seen in the rd1 photoreceptors [Sharma A K, Rohrer B: Calcium-induced calpain mediates apoptosis via caspase-3 in a mouse photoreceptor cell line. J Biol Chem 2004, 279(34):35564-35572] In the rd1 mouse, Ca²⁺ influx is due to permanently opened cGMP-gated cation channels and it is the best characterized model for RP [Farber D B: From mice to men: the cyclic GMP phosphodiesterase gene in vision and disease. The Proctor Lecture. Invest Ophthalmol Vis Sci 1995, 36(2):263-275; Farber D B, Lolley R N: Cyclic guanosine monophosphate: elevation in degenerating photoreceptor cells of the C3H mouse retina. Science 1974, 186:449-451; Fox D A, Poblenz A T, He L: Calcium overload triggers rod photoreceptor apoptotic cell death in chemical-induced and inherited retinal degenerations. Ann NY Acad Sci 1999, 893:282-285]. Likewise, 661W cells challenged with hydroperoxides recapitulate many of the steps in cell death observed in the light-damaged albino mouse retina, a model for oxidative stress in AMD [14. Kunchithapautham K, Rohrer B: Apoptosis and Autophagy in Photoreceptors Exposed to Oxidative Stress. Autophagy 2007, 3(5)]. Both the light-damage and the rd1 mouse retina have been used to investigate neuroprotective therapies (see [15. Wenzel A, Grimm C, Samardzija M, Reme C E: Molecular mechanisms of light-induced photoreceptor apoptosis and neuroprotection for retinal degeneration. Prog Retin Eye Res 2005, 24(2):275-306] for a comprehensive summary), focusing predominantly on neurotrophins and antioxidants. Although the metabolic effects of calcium or oxidative stress have not been measured directly in the mouse retina, it was found that both rd1 retina and retina exposed to light-damage expressed high levels of stress and metabolic genes at onset of damage but expression of metabolic genes dropped in parallel with the loss of cells [Lohr H R, Kuntchithapautham K, Sharma A K, Rohrer B: Multiple, parallel cellular suicide mechanisms participate in photoreceptor cell death. Exp Eye Res 2006, 83(2):380-389].

3. Assays of Energy Metabolism

While genomic and proteomic analyses describe which molecules are present in a tissue, it is the recent advances in metabolomics that have provided measures of molecular activities that can be related to pathology [Lenz E M, Wilson I D: Analytical strategies in metabonomics. J Proteome Res 2007, 6(2):443-458; Nicholas P C, Kim D, Crews F T, Macdonald J M: (1)H NMR-Based Metabolomic Analysis of Liver, Serum, and Brain Following Ethanol Administration in Rats. Chem Res Toxicol 2007]. Metabolomic studies have shown that changes in energy metabolism are the earliest markers of cellular stress, which reflects its intimate linkage to so many biochemical processes (e.g., membrane integrity, ion balance, protein synthesis, etc.). It is evident that metabolomic measurements will profoundly enhance drug discovery but prior to the assays disclosed herein, assay methods were not amenable to high throughput assay platforms. Recently, two groups used the Cytosensor® microphysiometer to measure extracellular fluxes linked to energy metabolism and it was shown that glucose utilization via different metabolic pathways can be estimated from extracellular flux measurements with an accuracy comparable to the traditional radiometric assays ([Wiley C, Beeson C: Continuous measurement of glucose utilization in heart myoblasts. Anal Biochem 2002, 304(2):139-146; Eklund S E, Taylor D, Kozlov E, Prokop A, Cliffel D E: A microphysiometer for simultaneous measurement of changes in extracellular glucose, lactate, oxygen, and acidification rate. Anal Chem 2004, 76(3):519-527]; Appendix 2). Although the microphysiometer is not amenable to high-throughput, Seahorse Biosciences developed a multi-well plate version of the instrument (XF24) that measures extracellular fluxes for lactic acid and oxygen using a multi-well plate format (Am. J. Physiol. Cell Physiol. 2007, 292(1), C125-C136 and also Drug Discovery Today 2008, 13(5-6), 1-8).

### B. COMPOUNDS

The present invention is directed to a class of compounds, including the pharmaceutically acceptable salts of the compounds, having the structure of formula I:

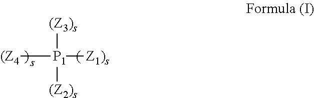

wherein:

P₁ is a pharmacophore where the structure of said pharmacophore comprises one or more hydrogen bond donor and/or acceptor groups;

Z₁ is -(L₁)_{s′}-P₂;

Z₂ is -(L₂)_{s′}-P₃-(L₃)_{s′}-P₄;

Z₃ is -(L₄)_{s′}-P₅;

Z₄ is -(L₅)_{s′}-P₅;

s and s′ are each independently an integer selected from 0 to 5;

P₂ is a pharmacophore where the structure of said pharmacophore comprises one or more hydrogen bond donor and/or acceptor groups; and/or one or more hydrophobic groups;

P₃ is a pharmacophore where the structure of said pharmacophore comprises one or more hydrogen bond donor and/or acceptor groups;

P₄ is a pharmacophore where the structure of said pharmacophore comprises one or more hydrophobic groups;

P₅ is a pharmacophore where the structure of said pharmacophore comprises one or more hydrophobic groups;

P₆ is a pharmacophore where the structure of said pharmacophore comprises one or more hydrophobic groups;

L₁, L₂, L₃, L₄ and L₅ are each independently a linker selected from the group consisting of —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰² wherein each of said group is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰²;

R¹⁰¹ and R¹⁰² are each independently selected from the group consisting of hydrogen, alkyl, alkenyl, alkynyl, cycloalkyl, aryl, heterocycloalkyl and heteroaryl; wherein each R¹⁰¹ and R¹⁰² is optionally independently substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl;

wherein said one or more hydrogen bond donor and/or acceptor groups, and said one or more hydrophobic groups are each optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰² wherein each of said substitutent is optionally independently substituted with one or more substitutents independently selected from the group consisting of hydrogen, halogen, cyano, —OR¹⁰¹, alkyl, alkenyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰²;

wherein each of L₁, L₂, L₃, L₄ and L₅ is optionally fused with the adjacent one or more pharmacophores.

In one embodiment of the invention, the compound of formula I, or a pharmaceutically acceptable salt thereof, is a compound wherein the hydrogen bond donor and/or acceptor groups comprises a mono-, a bi- or a tricyclic-heterocyclic rings wherein said bicyclic- or tricyclic-heterocyclic rings are fused or non-fused; or a group selected from the group consisting of —OR¹⁰¹, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —SR¹⁰¹, —S(O)₂R¹⁰², —SR¹⁰¹, and —S(O)₂NR¹⁰¹R¹⁰²; and the one or more hydrophobic groups are selected from the group consisting of alkyl, alkenyl, alkynyl, cycloalkyl, aryl, heterocycloalkyl and heteroaryl.

In another embodiment, the compound of formula I, or a pharmaceutically acceptable salt thereof, is a compound wherein: the one or more hydrogen bond donor and/or acceptor groups of said P₁ are connected with one or more carbon, nitrogen, sulfur or oxygen atoms to form a linear or ring structure; said P₂ is an unsaturated 5, 6 or 7-membered mono-heterocyclic ring; said P₃ is a structural moiety comprises —OR¹⁰¹, —SR¹⁰¹, C(O)NR¹⁰¹R¹⁰², —NR¹⁰¹R¹⁰², or —NR¹⁰¹C(O)R¹⁰²; said P₄ is an alkyl, cycloalkyl, or aryl; said P₅ is an alkyl, cycloalkyl, or aryl; and said P₆ is an alkyl, cycloalkyl, or aryl. In another embodiment, the compound of formula I, or a pharmaceutically acceptable salt thereof, is a compound wherein the compound of formula I is a 5, 6 or 7-membered unsaturated and conjugated heterocyclic ring with one or more substitutents on said ring. In another embodiment, the compound of formula I, or a pharmaceutically acceptable salt thereof, is a compound wherein the compound of formula I is a thiazole, oxazole, furan, thiophene, pyrrole, imidazole, pyrazole, isoxazole, isothiazole, oxidizole, triazole or triazole with one or more substitutents.

In another embodiment, the compound of formula I, or a pharmaceutically acceptable salt or a prodrug thereof, is a compound of formula II:

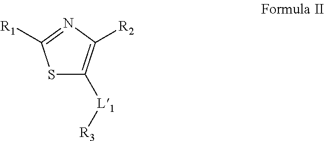

wherein,

R₁ is selected from a group consisting of aryl, —O—, heteroaryl such as pyridyl, pyrazyl, pyrimidinyl, and pyridazinyl, triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl;

R₂ is an alkyl, heteroaryl or aryl group;

L′₁ is a linker selected from the group consisting of alkyl, alkene, alkoxy, amine, imine, ester and amide;

R₃ is selected from the group consisting of aryl, heterocyclyl, amide, carboxylic acid, and carboxylic ester;

wherein each of R₁, R₂, L′₁ and R₃ is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms R₁ can be phenyl or pyridyl. In some forms R₂ can be phenyl or pyridyl. In some forms L′₁ can be an ester, for example, —C(O)O—, —C(O)OCH₂— or —C(O)OCH₂CH₂—. In some forms the ester is a prodrug, the prodrug can for example form a negatively charged acid intracellularly hydrolyzed from the ester. In some forms L′₁ can be C₁₋₃ alkyl, C₁₋₃ alkene or C₁₋₃ alkoxy. In some forms L′₁ can be an amide, for example —NHC(O)—, —C(O)NH—, —C(O)NH-alkyl- or -alkyl-NHC(O)—. In some forms the alkyl is C₁₋₃ alkyl. In some forms L′₁ can be an amine, for example —CH₂NH— or —CH═N—. In some forms R₃ can be phenyl or morpholine. In some forms L′₁ is optionally substituted with hydroxyl. In some forms R₁ can be phenyl or pyridyl, R₂ can be phenyl or pyridyl, L′₁ can be C₁₋₃ alkyl, C₁₋₃ alkene or C₁₋₃ alkoxy or an ester, for example, —C(O)O—, —C(O)OCH₂— or —C(O)OCH₂CH₂—.

In another embodiment, the compound of formula I, or a pharmaceutically acceptable salt thereof, is a bicyclic fused heterocyclic ring with one or more substitutents on either one or both of the fused rings. In another embodiment, the compound of formula I, or a pharmaceutically acceptable salt thereof, is an indole, oxindole, benzoimidazole, benzothiazole, benzoxazole, indazole, benzofuran, benzothiophene, purine, quinoline, isoquinoline, or cinnoline with one or more substitutents.

In another embodiment, the compound of formula I, or a pharmaceutically acceptable salt or a prodrug thereof, is a compound of formula III:

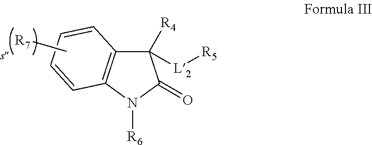

wherein,

R₄ is —OR¹⁰¹, —NR¹⁰¹R¹⁰², or —SR¹⁰¹;

R₅ is selected from a group consisting of pyridyl, pyrazyl, pyrimidinyl, and pyridazinyl, triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl;

R₆ is an alkyl or aryl group;

Each R₇ is individually a hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, or —S(O)₂NR¹⁰¹R¹⁰² which is attached to one or more positions of the phenyl ring;

L′₂ is a linker selected from the group consisting of alkyl, —C(═O), and amide;

S″ is an integer selected from 0 to 4;

wherein each of R₄, R₅, R₆, R₇, and L′₂ is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms R₇ can be hydrogen or halogen, R₆ can be C₁₋₆ alkyl, L′₂ can be alkyl, —C(═O), and amide, R₄ can be is —OR¹⁰¹ and R₅ can be pyridyl.

In another embodiment, the compound of formula I, or a pharmaceutically acceptable salt thereof, is a tricyclic fused heterocyclic ring with one or more substitutents on either one, two and/or three of the fused rings. In another embodiment, the compound of formula I, or a pharmaceutically acceptable salt thereof, is a 6,5,5-membered, 6,5,6-membered or 6,6,6-membered tricyclic fused heterocyclic ring with one or more substitutents on either one, two and/or three of the fused rings. The rings forming the triyclic heterocyclic ring can, for example, include phenyl, pyrrolidine and tetrahydrofuran.

In another embodiment, the compound of formula I, or a pharmaceutically acceptable salt or a prodrug thereof, is a compound of formula IV:

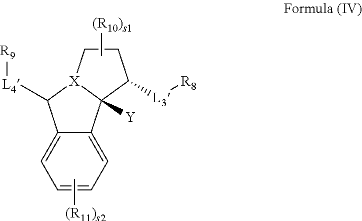

wherein,

X is CR₁₂ or N;

Y is —OR¹⁰¹, —NR¹⁰¹R¹⁰², —SR¹⁰¹ or halogen;

R₃ and R₉ are each independently selected from a group consisting of hydrogen, alkyl, cycloalkyl, aryl, heterocycloalkyl, and heteroaryl;

R₁₀ and R₁₁ are each independently selected from a group consisting of hydrogen, halogen, cyano, —OR¹⁰¹, —SR¹⁰¹, —NR¹⁰¹R¹⁰², alkyl, cycloalkyl, aryl, heterocycloalkyl, and heteroaryl;

L′₃ and L′₄ are each independently a linker selected from the group consisting of —OR¹⁰¹, —SR¹⁰¹, alkyl, cycloalkyl, —C(O)R¹⁰¹, —C(O)OR¹⁰¹, —C(O)NR¹⁰¹R¹⁰², and —NR¹⁰¹R¹⁰²;

S1 is a subscript selected from 0 to 2;

S2 is a subscript selected from 0 to 4;

R₁₂ is selected from a group consisting of hydrogen, alkyl, cycloalkyl, cycloalkenyl, aryl, heterocycloalkyl, and heteroaryl;

wherein each of R₈, R₉, R₁₀, R₁₁, R₁₂, L′₃, L′₄ and Y is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms X can be N, Y can be —OR¹⁰¹, for example —OH, L′₄ can be C₁₋₆alkyl, R₉ can be hydrogen, phenyl, furanyl, cyclopentadienyl or imidazole, R₁₀ can be hydrogen, R₁₁ can be hydrogen, L′₃ can be C₁₋₆ alkyl, and R₈ can be pyridyl.

In another embodiment, the compound of formula I, or a pharmaceutically acceptable salt or a prodrug thereof, is a compound of formula V:

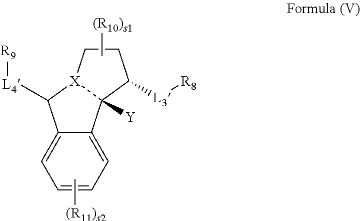

wherein,

X is CR₁₂, NR₁₂, SiR₁₂, O, S, P or B;

Y is a mono-heterocyclic ring, a bicyclic- or a tricyclic-heterocyclic rings wherein said bicyclic- or tricyclic-heterocyclic rings are fused or non-fused; or a group selected from the group consisting of —OR¹⁰¹, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —S(O)₂NR¹⁰¹R¹⁰² and halogen;

R₈ and R₉ is each independently selected from a group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰²;

Each R₁₀ is individually a hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, or —S(O)₂NR¹⁰¹R¹⁰² which is attached to one or more positions of formula I other than the phenyl ring;

Each R₁₁ is individually a hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, or —S(O)₂NR¹⁰¹R¹⁰² which is attached to one or more positions of the phenyl ring;

L′₃ and L′₄ are each independently a linker selected from the group consisting of —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰² wherein each of said group is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰²;

S1 and S2 are each independently an integer selected from 0 to 4;

R₁₂ is selected from a group consisting of hydrogen, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰²;

the dotted line between X and the carbon atom connected to Y represents a covalent bond between X and said carbon atom or the absence of said covalent bond;

wherein each of R₈, R₉, R₁₀, R₁₁, R₁₂, and Y is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms X can be N, Y can be —OR¹⁰¹, for example —OH, L′₄ can be C₁₋₆ alkyl, R₉ can be hydrogen, phenyl, furanyl, cyclopentadienyl or imidazole, R₁₀ can be hydrogen, R₁₁ can be hydrogen, L′₃ can be C₁₋₆ alkyl, and R₈ can be pyridyl.

In another embodiment, the compound of formula I, or a pharmaceutically acceptable salt or a prodrug thereof, is a compound of formula VI:

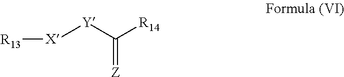

wherein,

R₁₃ and R₁₄ is each independently selected from a group consisting of cycloalkyl, cycloalkenyl, aryl, heterocycloalkyl, and heteroaryl;

X′ and Y′ is each independently selected from a group consisting of CR₁₂, NR₁₂, O and S;

Z is O, S, CR₁₂ or NR₁₂;

R₁₂ is selected from a group consisting of hydrogen, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰²;

wherein each of R₁₂, R₁₃, R₁₄, X′, Y′, and Z is optionally independently substituted with one or more substitutents independently selected from the group consisting of hydrogen, halogen, cyano, C(═O), —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms R₁₃ and R₁₄ can each independently be phenyl, cyclopentene, X′ and Y′ can each independently be NR₁₂.

In another embodiment, the compound of formula I, or a pharmaceutically acceptable salt or a prodrug thereof, is a compound of formula VII:

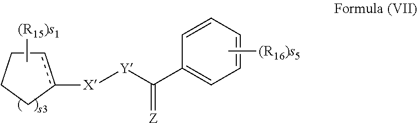

wherein,

Each R₁₅ is independently a hydrogen, halogen, cyano, C(═O), —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, or —S(O)₂NR¹⁰¹R¹⁰² which is attached to one or more positions of the left ring of formula VII;

Each R₁₆ is independently a hydrogen, halogen, cyano, C(═O), —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, or —S(O)₂NR¹⁰¹R¹⁰² which is attached to one or more positions of the phenyl ring of formula VII;

X′ and Y′ is each independently selected from a group consisting of CR₁₂, NR₁₂, O and S;

Z is O, S, CR₁₂ or NR₁₂;

S4 and S5 are each independently an integer selected from 0 to 5;

S3 an integer selected from 1 to 3; and the dotted line in the left ring of formula VII represents a double bond or the absence of said double bond.

In some forms, X′ and Y′ can each independently be NR₁₂, for example H, benzyl, C₁₋₆ alkyl, methylene pyridyl, S3 can be an integer of 1 or 2, and R₁₅ and R₁₆ can be hydrogen.

In another embodiment, the compound of formula I, or a pharmaceutically acceptable salt or a prodrug thereof, is a compound of formula VIII:

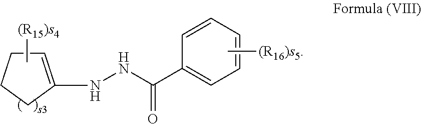

Each R₁₅ is independently a hydrogen, halogen, cyano, C(═O), —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, or —S(O)₂NR¹⁰¹R¹⁰² which is attached to one or more positions of the left ring of formula VII;

Each R₁₆ is independently a hydrogen, halogen, cyano, C(═O), —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, or —S(O)₂NR¹⁰¹R¹⁰² which is attached to one or more positions of the phenyl ring of formula VIII;

S4 and S5 are each independently an integer selected from 0 to 5; and

S3 an integer selected from 1 to 3.

In some forms, S3 can be an integer of 1 or 2, and R₁₅ and R₁₆ can be hydrogen.

In another embodiment, the compound of formula I, or a pharmaceutically acceptable salt or a prodrug thereof, is a compound of formula IX:

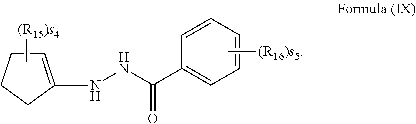

Each R₁₅ is independently a hydrogen, halogen, cyano, C(═O), —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, or —S(O)₂NR¹⁰¹R¹⁰² which is attached to one or more positions of the left ring of formula VII;

Each R₁₆ is independently a hydrogen, halogen, cyano, C(═O), —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, or —S(O)₂NR¹⁰¹R¹⁰² which is attached to one or more positions of the phenyl ring of formula VIII; and

S4 and S5 are each independently an integer selected from 0 to 5.

In another embodiment, the compound of formula I, or a pharmaceutically acceptable salt or a prodrug thereof, is a compound of formula X:

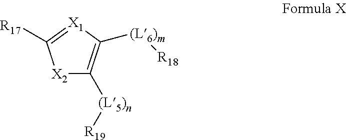

wherein,

X₁ can be N or CH;

X₂ can be O, NR¹⁰¹, Se, CH₂;

R₁₇ is selected from a group consisting of aryl, heteroaryl, such as pyridyl, pyrazyl, pyrimidinyl, and pyridazinyl, triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl;

R₁₈ and R₁₉ are independently selected from a group consisting of an alkyl, heteroaryl, aryl, heterocyclyl, amide, carboxylic acid, and carboxylic ester;

L′₅ and L′₆ are linkers independently selected from the group consisting of alkyl, alkene, alkoxy, amine, ester and amide;

m can be an integer of 0 or 2;

n can be an integer of 0 or 2;

wherein each of R₁₇, R₁₈, L′₅, L′₆ and R₁₉ is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms of Formula X, R₁₇ can be phenyl or pyridyl. In some forms R₁₈ or R₁₉ can independently be phenyl, morpholine or pyridyl. In some forms L′₅ or L′₆ can independently be an ester, for example, —C(O)O—, —C(O)OCH₂— or —C(O)OCH₂CH₂—. In some forms the ester is a prodrug, the prodrug can for example form a negatively charged acid intracellularly from the ester. In some forms L′₅ or L′₆ can independently be C₁₋₃ alkyl, C₁₋₃ alkene or C₁₋₃ alkoxy. In some forms L′₅ or L′₆ can independently be an amide, for example —NHC(O)—, —C(O)NH—, —C(O)NH-alkyl- or -alkyl-NHC(O)—. In some forms the alkyl is C₁₋₃ alkyl. In some forms L′₅ or L's can independently be an amine, for example —CH₂NH— or —CH═N—. In some forms L′₅ or L′₆ can independently can optionally be substituted with hydroxyl. In some forms n is 0 when m is 1. In some forms n is 1 when m is 0. In some forms n is 1 when m is 1. In some forms X₁ can be N when X₂ is S. In some forms X₁ can be N when X₂ is Se. In some forms X₁ can be N when X₂ is O. In some forms X₁ can be N when X₂ is CH₂. In some forms, R₁₈ or R₁₉ can independently be phenyl, morpholine or pyridyl, L′₅ or L's can independently be C₁₋₃ alkyl, C₁₋₃ alkene or C₁₋₃ alkoxy or an ester, for example, —C(O)O—, —C(O)OCH₂— or —C(O)OCH₂CH₂—, X₁ can be N when X₂ is S, X₁ can be N when X₂ is Se and n is 0 when m is 1.

In another embodiment, the compound of formula I, or a pharmaceutically acceptable salt or a prodrug thereof, is a compound of formula XI:

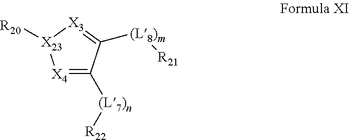

wherein

X₃ and X₄ independently can be N or CH;

X₂₃ can be N or CH;

R₂₀ is selected from a group consisting of aryl, heteroaryl such as pyridyl, pyrazyl, pyrimidinyl, and pyridazinyl, triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl;

R₂₁ and R₂₂ are independently selected from a group consisting of an alkyl, heteroaryl, aryl, heterocyclyl, amide, carboxylic acid, and carboxylic ester;

L′₇ and L′₈ are linkers independently selected from the group consisting of alkyl, alkene, alkoxy, amine, ester and amide;

m can be an integer 0 or 2;

n can be an integer 0 or 2;

wherein each of R₂₀, R₂₁, L′₇, L′₈ and R₂₂ is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms of Formula XI R₂₀ can be phenyl or pyridyl. In some forms R²¹ or R²² can independently be phenyl, morpholine or pyridyl. In some forms L′₇ or L′₈ can independently be an ester, for example, —C(O)O—, —C(O)OCH₂— or —C(O)OCH₂CH₂—. In some forms the ester is a prodrug, the prodrug can for example form a negatively charged acid intracellularly from the ester. In some forms L′₇ or L′₈ can independently be C₁₋₃alkyl, C₁₋₃ alkene or C₁₋₃ alkoxy. In some forms L′₇ or L′₈ can independently be an amide, for example —NHC(O)—, —C(O)NH—, —C(O)NH-alkyl- or -alkyl-NHC(O)—. In some forms the alkyl is C₁₋₃ alkyl. In some forms L′₇ or L′₈ can independently be an amine, for example —CH₂NH— or —CH═N—. In some forms L′₅ or L′₆ can independently can optionally be substituted with hydroxyl. In some forms n is 0 when m is 1. In some forms n is 1 when m is 0. In some forms n is 1 when m is 1. In some forms X₃ and X₄ can be N when X₂₃ is CH. In some forms X₃ and X₄ can be CH when X₂₃ is CH. In some forms X₃ and X₄ can be N when X₂₃ is N. In some forms R₂₀ can be phenyl or pyridyl, R₂₁ or R₂₂ can independently be phenyl, morpholine or pyridyl, L′₇ or L′₈ can independently be C₁₋₃ alkyl, C₁₋₃ alkene C₁₋₃ alkoxy or an ester, for example, —C(O)O—, —C(O)OCH₂— or —C(O)OCH₂CH₂— and n is 0 when m is 1.

In another embodiment, the compound of formula I, or a pharmaceutically acceptable salt or a prodrug thereof, is a compound of formula XII:

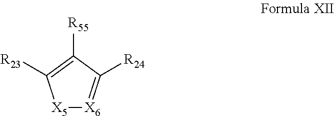

wherein

X₅ can be O, S, NR₅₁ or CH₂;

X₆ can be N or CH;

R₅₁ can be hydrogen, alkyl, alkene, aryl, heteraryl, heterocyclyl, silyl, silane, ester, ketone, carboxylic acid or -L′₂-R₅₂;

L′₁₂ can be alkyl, alkene, ketone, alkoxy, amine, ester and amide;

R₅₂ can be hydrogen, aryl, heteroaryl, heterocyclyl or cycloalkyl;

R₂₃ can be hydrogen, aryl, heteraryl, heterocyclyl, silyl, silane, ester, ketone, carboxylic acid or L′₁₃-R₅₃;

L′₁₃ can be alkyl, alkene, ketone, alkoxy, amine, ester and amide;

R₅₃ can be hydrogen, aryl, heteroaryl, heterocyclyl or cycloalkyl;

R₂₄ can be hydrogen, aryl, heteraryl, heterocyclyl, silyl, silane, ester, ketone, carboxylic acid or L′₁₄-R₅₄;

L′₁₄ can be alkyl, alkene, ketone, alkoxy, amine, ester and amide;

R₅₄ can be hydrogen, aryl, heteroaryl, heterocyclyl or cycloalkyl;

R₅₅ can be hydrogen, aryl, heteraryl, heterocyclyl, silyl, silane, ester, ketone, carboxylic acid or -L′₁₅-R₅₆;

L′₁₅ can be alkyl, alkene, ketone, alkoxy, amine, ester and amide;

R₅₆ can be hydrogen, aryl, heteroaryl, heterocyclyl or cycloalkyl;

wherein only one of R₂₃, R₂₄ and R₅₅ can be hydrogen;

wherein each of R₂₃, R₂₄, L′₁₂, L′₁₃, L′₁₄, L′₁₅, R₅₁, R₅₂, R₅₃, R₅₄, R₅₅, and R₅₆ is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms of Formula XII X₅ can be NR₅₁ when X₅ is N. In some forms R₅₁ can be hydrogen, phenyl, substituted pymidinone, pyridyl, methyl, acetophenyl, acetoaryl, acetoheteroaryl, benzylic, heteroaryl methylene or -L′₁₂-R₅₂. In some forms -L′₁₂ can be C₁₋₃ alkyl, C₁₋₃ alkene, C₁₋₃ alkoxy, C₁₋₃ alkyl ketone. In some forms R₅₂ can be phenyl, substituted pymidinone, pyridyl, methyl, acetophenyl, acetoaryl, acetoheteroaryl, benzylic, heteroaryl methylene.

In some forms L′₁₃, L′₁₄, L′₁₅ can independently be C₁₋₃ alkyl, C₁₋₃ alkene, C₁₋₃ alkoxy, amide-alkyl (for example, —C(O)NHCH₂—).

In some forms R₅₃, R₅₄ and R⁵⁶ can be hydrogen, phenyl, cyclohexane, morpholine substituted cyclohexane, thiazole, morpholine, furan, pyridyl, alkyl substituted pyrimidinone, alkoxy substituted phenyl, ester, for example C(O)OCH₂CH₃, trisubstituted silane, for example trimethyl silane.

In some forms R₂₃, R₂₄ and R₅₅ can independently be hydrogen, phenyl, cyclohexane, morpholine substituted cyclohexane, thiazole, morpholine, furan, pyridyl, alkyl substituted pyrimidinone, alkoxy substituted phenyl, ester, for example C(O)OCH₂CH₃, trisubstituted silane, for example trimethyl silane.

In some forms X₅ can be NR₅₁ and X₆ can be N, forms R₅₁ can be hydrogen, phenyl, substituted pymidinone pyridyl or -L′₁₂-R₅₂, -L′₁₂ can be C₁₋₃ alkyl, C₁₋₃ alkene, C₁₋₃alkoxy, C₁₋₃ alkyl ketone, R₅₂ can be phenyl, substituted pymidinone, pyridyl or methyl, L′₁₃, L′₁₄, L′₁₅ can independently be C₁₋₃ alkyl, C₁₋₃ alkene, C₁₋₃ alkoxy, amide-alkyl (for example, —C(O)NHCH₂—), R₅₃, R₅₁ and R₅₆ can be hydrogen, phenyl, cyclohexane, morpholine substituted cyclohexane, thiazole, morpholine, furan, pyridyl, alkyl substituted pyrimidinone, alkoxy substituted phenyl, ester, for example C(O)OCH₂CH₃, trisubstituted silane, for example trimethyl silane, and R₂₃, R₂₄ and R₅₅ can independently be hydrogen, phenyl, cyclohexane, morpholine substituted cyclohexane, thiazole, morpholine, furan, pyridyl, alkyl substituted pyrimidinone, alkoxy substituted phenyl, ester, for example C(O)OCH₂CH₃, trisubstituted silane, for example trimethyl silane.

In another embodiment, the compound of formula I, or a pharmaceutically acceptable salt or a prodrug thereof, is a compound of formula XIII:

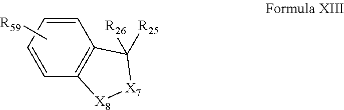

wherein

X₇ can be S, Se, C(O) or O;

X₈ can be NR₅₇ or CHR₅₈;

Each R₅₉ can independently be hydrogen, halogen, C₁₋₃ alkyl or C₁₋₃ alkoxy, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, or —S(O)₂NR¹⁰¹R¹⁰² which is attached to one or more positions of the phenyl ring;

R₂₅ can be alkyl, alkoxy —OR¹⁰¹, —NR¹⁰¹R¹⁰², or —SR¹⁰¹

R₂₆ can be alkyl, alkoxy, pyridyl, pyrazyl, pyrimidinyl, and pyridazinyl, triazolyl, imidazolyl, furanyl, thiophenyl, pyrazolyl, oxazolyl, isoxazolyl, thiazolyl, 1,2,3-, 1,2,4-, 1,2,5-, 1,3,4-oxadiazolyl and isothiazolyl or -(L′₁₇)₀₋₂-R₆₁

R₅₇ and R₅₈ are independently selected from a group consisting of alkyl, phenyl methylene, aryl methylene, alkaloid methylene, pyridyl, pyrazyl, pyrimidinyl, and pyridazinyl, triazolyl, imidazolyl, furanyl, thiophenyl, pyrazolyl, oxazolyl, isoxazolyl, morpholine thiazolyl, 1,2,3-, 1,2,4-, 1,2,5-, or 1,3,4-oxadiazolyl, isothiazolyl or -L′₁₆-R⁶⁰—;

L′₁₆ and L′₁₇ are linkers and independently selected from the group consisting of alkyl, —C(═O), and amide;

R₆₀ and R₆₁ can independently be alkyl, aryl, heterocyclyl, phenyl methylene, aryl methylene, alkaloid methylene, heteroaryl, such as pyridyl, pyrazyl, pyrimidinyl, and pyridazinyl, triazolyl, imidazolyl, furanyl, thiophenyl, pyrazolyl, oxazolyl, isoxazolyl, morpholine thiazolyl, 1,2,3-, 1,2,4-, 1,2,5-, or 1,3,4-oxadiazolyl, isothiazolyl;

R₂₆ and R₂₅ optionally together form a doublebond, for example ═O, ═NR₇₀, ═S or ═C;

R₇₀ can be alkyl, benzyl or pyridyl;

R₂₆ and R₂₅ optionally form a substituted or unsubstituted fused ring or cyclic moiety;

wherein each of R₂₅, R₂₆, R₅₇, R₅₈, R₅₉, R₆₀, R₆₁ L′₁₆ and L′₁₇ is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms of Formula XIII R₂₆ and R₂₅ together form a doublebond, for example ═O. In some forms R₂₆ and R₂₅ combines to form a cyclic moiety, for example, heterocyclic, cycloalkyl, aryl, heteroaryl. In some forms the cyclic moisty can be a dioxane moiety further substituted at 1, 2 or 3 positions with an alkyl or phenyl. In some forms X₇ is C(O). In some forms R₅₇ and R₅₈ are independently alkyl, phenyl or morpholine. In some forms R₂₆ and R₂₅ together form C═O, R₅₉ can be hydrogen, X₇ can be S, Se, C(O) or O; X₈ can be NR₅₇ or CHR₅₈. In some forms R₂₆ and R₂₅ together form a, R₅₉ can be hydrogen, X₇ can be S, Se, C(O) or O; X₅ can be NR₅₇ or CHR₅₈. In some forms R₂₆ and R₂₅ together form a dioxane moiety further substituted at 1, 2 or 3 positions with an alkyl or phenyl, R₅₉ can be hydrogen, X₇ can be S, Se, C(O) or O; X₈ can be NR₅₇ or CHR₅₈.

In another embodiment, the compound of formula I, or a pharmaceutically acceptable salt or a prodrug thereof, is a compound of formula XIV:

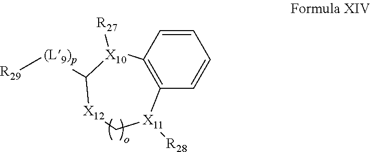

wherein

X₁₀ and X₁₁ can independently be N or CH;

X₁₂ can be O or C(O);

R₂₇ can be hydrogen, alkyl, alkoxy —OR¹⁰¹, —NR¹⁰¹R¹⁰², or —SR¹⁰¹:

R₂₈ can be hydrogen, alkyl, alkoxy —OR¹⁰¹, —NR¹⁰¹R¹⁰², or —SR¹⁰¹;

L′₉ can be alkyl, alkene, alkoxy, amine, ketone, ester or amide;

R₂₉ can be aryl, heteroaryl, such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl and isothiazolyl;

o can be an integer of 0 or 2;

p can be an integer of 0 or 2;

wherein each of R₂₇, R₂₈, R₂₉ and L′₉ is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms o can be 1. In some forms o can be 0. In some forms p can be 1. In some forms p can be 0. In some forms R₂₇ can be hydroxyl when X₁₀ is CH. In some forms R₂₈ can be C₁₋₆ alkyl. In some forms R₂₈ can be C₄ alkyl. In some forms R₂₉ can be pyridyl. In some forms L's can be C₁₋₃ alkyl or C₁₋₃ alkyl ketone.

In some forms o can be an integer of 1, p can be an integer of 1 R₂₇ can be hydroxyl when X₁₀ is CH, R₂₈ can be C₄ alkyl, R₂₉ can be pyridyl and L′₉ can be C₁₋₃ alkyl or C₁₋₃ alkyl ketone.

In another embodiment, the compound of formula I, or a pharmaceutically acceptable salt or a prodrug thereof, is a compound of formula XV:

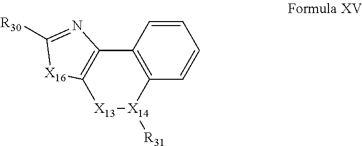

wherein

X₁₃ can be CH₂, O or C(O);

X₁₄ can be CH or N;

X₁₆ can be S, O, Se or CH₂;

R₃₀ can be aryl, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl and isothiazolyl;

R₃₁ can be aryl, heteroaryl, phenyl, pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl and isothiazolyl;

R₃₁ can optionally be combined with X₁₃ to form a ring moiety;

wherein each of R₃₀ and R₃₁ is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms X₃₁ can be C(O). In some forms R₃₁ can be aryl or phenyl. In some forms R₃₀ can be heteroaryl or pyridyl. In some form X₁₆ can be S. In some form X₁₆ can be O In some form X₁₆ can be Se. In some forms R₃₁ is combined with X₁₃ to form heterocyclic moiety. In some forms the heterocyclic moiety can be a multiring moiety. In some forms the ring moiety can be a benzoxazin moiety.

In some forms R₃₁ can be phenyl, X₁₆ can be O, S or Se, R₃₀ can be pyridyl.

In another embodiment, the compound of formula I, or a pharmaceutically acceptable salt or a prodrug thereof, is a compound of formula XVI:

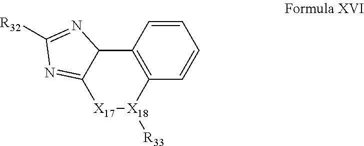

wherein

X₁₇ can be CH₂, O or C(O);

X₁₈ can be CH or N;

R₃₂ can be aryl, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl and isothiazolyl;

R₃₃ can be aryl, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl and isothiazolyl;

R₃₃ can optionally be combined with X₁₇ to form a ring moiety;

wherein each of R₃₂ and R₃₃ is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms X₁₇ can be C(O). In some forms R₃₃ can be aryl or phenyl. In some forms R₃₂ can be heteroaryl or pyridyl. In some forms R₃₃ is combined with X₁₃ to form heterocyclic moiety. In some forms the heterocyclic moiety can be a multiring moiety. In some forms the ring moiety can be a benzoxazin moiety.

In some forms R₃₃ can be phenyl or C(O), R₃₂ can be pyridyl.

In another embodiment, the compound of formula I, or a pharmaceutically acceptable salt or a prodrug thereof, is a compound of formula XVII:

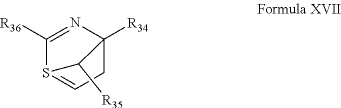

wherein

R₃₄ can be aryl, heteroaryl, phenyl, pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl and isothiazolyl;

R₃₆ can be aryl, heteroaryl, phenyl, pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl and isothiazolyl;

R₃₅ can be aryl, heteroaryl, phenyl, pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl, isothiazolyl or (L′₁₈)₀₋₂-R₆₂;

L′₁₈ can be alkyl, alkene, alkoxy, amine, ketone, ester or amide;

R₆₂ can be aryl, heteroaryl, phenyl, pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl or isothiazolyl;

wherein each of R₃₁, R₃₅, R₃₆, R₆₂ and L′₁ s is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms R₃₄ can be aryl or phenyl. In some forms R₃₆ can be heteroaryl or pyridyl. In some forms L′₁₈ can be C₁₋₃ alkyl or amide, for example —CH₂C(O)NH—. In some forms R₆₂ can be aryl or phenyl.

In some forms R₃₄ can be phenyl, R₃₅ can be (L′₁₈)₀₋₂-R₆₂, and R₆₂ can be phenyl.

In another embodiment, the compound of formula I, or a pharmaceutically acceptable salt or a prodrug thereof, is a compound of formula XVIII:

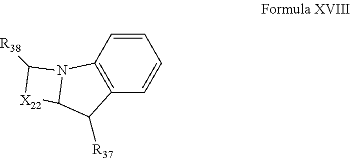

wherein

X₂₂ can be O or Se;

R₃₇ can be aryl, heteroaryl, phenyl, pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl, isothiazolyl or (L′₁₉)₀₋₂-R₆₃;

L′₁₉ can be alkyl, alkene, alkoxy, amine, ketone, ester or amide;

R₆₃ can be aryl, heteroaryl, phenyl, pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl or isothiazolyl;

R₃₈ can be aryl, heteroaryl, phenyl, pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl or isothiazolyl;

wherein each of R₃₇, R₃₅, R₃₈, R₆₃ and L′₁₉ is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms X₂₂ can be O. In some forms R₃₈ can be heteroaryl or pyridyl. In some forms L′₁₉ can be C₁₋₃ alkyl amine or amide, for example —CH₂NH— or —CH₂C(O)NH—. In some forms R₆₃ can be aryl or phenyl.

In some forms X₂₂ can be O, R³⁸ can be pyridyl, R₃₇ can be (L′₁₉)₀₋₂-R₆₃.

In another embodiment, the compound of formula I, or a pharmaceutically acceptable salt or a prodrug thereof, is a compound of formula XIX:

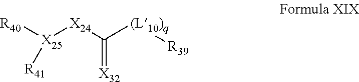

wherein

X₂₄ can be —S—, —O—, alkyl, alkene, alkoxy, amine, ketone, ester or amide;

X₂₅ can be aryl, heteroaryl, cycloalkyl, heterocyclyl, alkyl, alkene, alkoxy, amine, ketone, ester or amide;

R₄₀ can be hydrogen, aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl or isothiazolyl;

R₄₁ can be hydrogen, aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl or isothiazolyl;

X₃₂ and its double bond is either present or absent;

If present X₃₂ can be O or S;

L′₁₀ can be alkyl, alkene, alkoxy, amine, ketone, ester or amide;

R₃₉ can be hydrogen, aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl or isothiazolyl;

q is 0-3;

wherein each of R₃₉, R₄₀, R₄₁, X₂₄, X₂₅ and L′₁₀ is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms of Formula XIX X₂₄ and X₂₅ can independently be substituted with benzyl or phenyl. In some forms X₃₂ is present. In some forms X₃₂ is O. In some forms either R₄₀ or R₄₁ can be hydrogen. In some forms either R₄₀ or R₄₁ can be pyridyl. In some forms q is 1. In some forms q is 0. In some forms R₃₉ can be aryl or phenyl. In some forms L′₁₀ can be C₁₋₃ alkyl or amine, for example —NH— —CH₂NH— —CH₂CH₂— or —CH₂—. In some forms X₂₄ can be C₁₋₆ alkyl. In some forms X₂₅ can be C₁₋₆ alkyl.

In some forms X₃₂ can be O, X₂₅ can be substituted with benzyl or phenyl, X₂₄ can be alkyl, X₂₅ can be aryl, R₄₀ and R₄₁ can be hydrogen.

In another embodiment, the compound of formula I, or a pharmaceutically acceptable salt or a prodrug thereof, is a compound of formula XX:

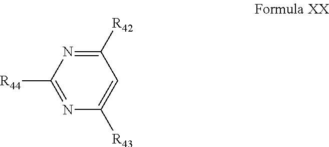

wherein

R₄₂ can be aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl or isothiazolyl;

R₄₄ can be aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl or isothiazolyl;

R₄₃ can be aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl isothiazolyl or L′₂₀-R₆₄;

L′₂₀ can be alkyl, alkene, alkoxy, amine, ketone, ester or amide;

R₆₄ can be hydrogen, aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl or isothiazolyl;

wherein each of R₄₂, R₄₃, R₄₄, R₆₄, and L′₂₀ is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms R₄₄ can be heteroaryl or pyridyl. In some forms L′₂₀ can be C₁₋₃ alkyl amine or amide, for example —CH₂NH— or —CH₂C(O)NH—. In some forms R₅₆ can be aryl or phenyl. In some forms R₄₂ can be aryl or phenyl.

In some forms R₄₂ can be aryl, for example phenyl, R₄₃ can be L′₂₀-R₆₄, and R₄₄ can be heteroaryl, for example pyridyl.

In another embodiment, the compound of formula I, or a pharmaceutically acceptable salt or a prodrug thereof, is a compound of formula XXI:

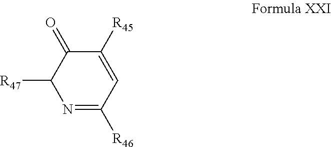

wherein

R₄₅ can be aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl or isothiazolyl;

R₄₇ can be aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl or isothiazolyl;

R₄₆ can be aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl, isothiazolylor L′₂₁-R⁶⁵;

L′₂₁ can be alkyl, alkene, alkoxy, amine, ketone, ester or amide;

R₆₅ can hydrogen, aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl or isothiazolyl;

wherein each of R₄₅, R₄₆, R₄₇, R₆₅, and L′₂₁ is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms R₄₇ can be heteroaryl or pyridyl. In some forms L′₂₁ can be C₁₋₃ alkyl, amine or amide, for example —CH₂NH— or —CH₂C(O)NH—. In some forms R₆₅ can be aryl or phenyl. In some forms R₄₅ can be aryl or phenyl.

In some forms R₄₅ can be aryl, for example phenyl, R₄₆ can be L′₂₁-R₆₅, and R₄₇ can be heteroaryl, for example pyridyl.

In another embodiment, the compound of formula I, or a pharmaceutically acceptable salt or a prodrug thereof, is a compound of formula XXII:

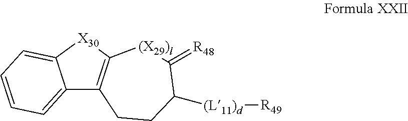

wherein

X₃₀ can be O, S or N-L′₂₂-R₆₆;

L′₂₂ can be present or absent;

If present L′₂₂ can be alkyl, alkene, alkoxy, amine, ketone, ester or amide;

R₆₆ can be hydrogen, alkyl, alkene, alkoxy, amine aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl or isothiazolyl;

X₂₉ can be O or CH₂;

l can be 0 or 2;

R₄₈ can be O or S;

L′₁₁ can be alkyl, alkene, alkoxy, amine, ketone, ester or amide;

d can be 0 or 2;

R₄₉ can be hydrogen, aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, 1,2,3-, 1,2,4-, 1,2,5-, or 1,3,4-oxadiazolyl or isothiazolyl;

wherein each of R₄₉, R₆₆, L′₁₁ and L′₂₂, is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms L′₁₁ can be C₁₋₃ alkyl, C₁₋₃ alkene, ketone, amine or amide, for example —CH₂—, —C(O)—CH₂NH— or —CH₂C(O)NH—. In some forms R₄₉ can be aryl, phenyl, C₁₋₆ alkyl. IN some forms L′₂₂ can be C₁₋₃ alkyl, C₁₋₃ alkene, ketone, amine or amide, for example —CH₂—, —C(O)—CH₂NH— or —CH₂C(O)NH—. In some forms L′₂₂ can be ketone. In some forms R₆₆ can be aryl, phenyl, C₁₋₆ alkyl.

In some forms X₃₀ can be N-L′₂₂-R₆₆, R₄₈ can be O, L′₁₁ can be C₁₋₃ alkyl or C₁₋₃ alkene.

In another embodiment, the compound of formula I, or a pharmaceutically acceptable salt or a prodrug thereof, is a compound of formula XXIII:

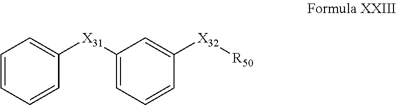

wherein

X₃₁ and X₃₂ can independently be amine or amide, for example —NH— or —NHC(O)—;

R₅₀ can be aryl, heteroaryl, heterocyclyl, cycloalkyl, phenyl, halo substituted phenyl, dimethyl cyclohexenone;

wherein each of R₅₀, X₃₁ and X₃₂ is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms X₃₁ and X₃₂ can both be —NH—. In some forms X₃₁ and X₃₂ can both be —NHC(O)—. In some forms R₅₀ can be fluoro substituted phenyl. In some forms R₅₀ can be dimethyl cyclohexenone.

In some forms X₃₁ and X₃₂ can be NH, R₅₀ can be dimethyl cyclohexenone or fluoro substituted phenyl.

In another embodiment, the compound of formula I, or a pharmaceutically acceptable salt or a prodrug thereof, is a compound of formula XXIV:

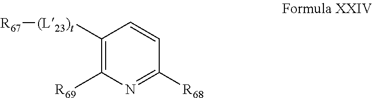

wherein

R₆₇ can be hydrogen, aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, thiophene, benzothophene, dibenzothiophene, 1,2,3-, 1,2,4-, 1,2,5-, or 1,3,4-oxadiazolyl or isothiazolyl;

L′₂₃ can be alkyl, alkene, alkoxy, amine, ketone, ester or amide;

t can be an integer of 0 or 2;

R₆₈ can be aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, thiophene, benzothophene, dibenzothiophene, 1,2,3-, 1,2,4-, 1,2,5-, or 1,3,4-oxadiazolyl or isothiazolyl;

R₆₉ can be aryl, cycloalkyl, heterocyclyl, alkyl, alkene, alkoxy, amine, ketone, ester amide, phenyl, heteroaryl such as pyridyl, pyrazyl, pyrimidinyl, and pyridazinyl, triazolyl, imidazolyl, furanyl, thiophenyl, morpholine, pyrazolyl, oxazolyl, isoxazolyl, thiazolyl, thiophene, benzothiophene, dibenzothiophene, 1,2,3-, 1,2,4-, 1,2,5-, or 1,3,4-oxadiazolyl or isothiazolyl;

wherein each of R₆₇, R₆₈, R₆₉ and L′₂₃ is optionally independently substituted with one or more substitutents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰².

In some forms R⁶⁷ can be heteroaryl, substituted or unsubstituted pyrazole or C₁₋₆ alkyl. In some forms R₆₇ can be dimethyl pyrazole or C₁₋₃ alkyl. In some forms each L′₂₃ can independently be alkyl or amide. In some forms R₆₈ can be heterocyclyl or heteroaryl, thiophene, benzothiophene or dibenzothiophene. In some forms R₆₉ can be heterocyclyl or morpholine.

In some forms R₆₇ can be dimethyl pyrazole or C₁₋₃ alkyl, L′₂₃ can independently be alkyl or amide, R₆₈ can be heterocyclyl or heteroaryl such as thiophene, benzothiophene or dibenzothiophene and R₆₉ can be heterocyclyl such as morpholine.

In another embodiment, the compounds of formula I, or a pharmaceutically acceptable salt or a prodrug thereof, is a compound selected from the group consisting of:

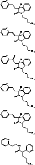 [SMILES: CCCCN1C(=O)C(O)(CCc2ccccn2)c2ccccc21 ; CCCCN1C(=O)C(O)(/C=C/c2ccccn2)c2ccccc21 ; CCCCC1C(=O)C(O)(CC(=O)c2ccccn2)c2ccccc21 ; CCCCC1C(=O)C(O)(CCc2ccccn2)c2ccccc21 ; CCCCC1C(=O)C(O)(/C=C/c2ccccn2)c2ccccc21 ; CCCCC(C(=O)NCCc1ccccn1)c1ccccc1]

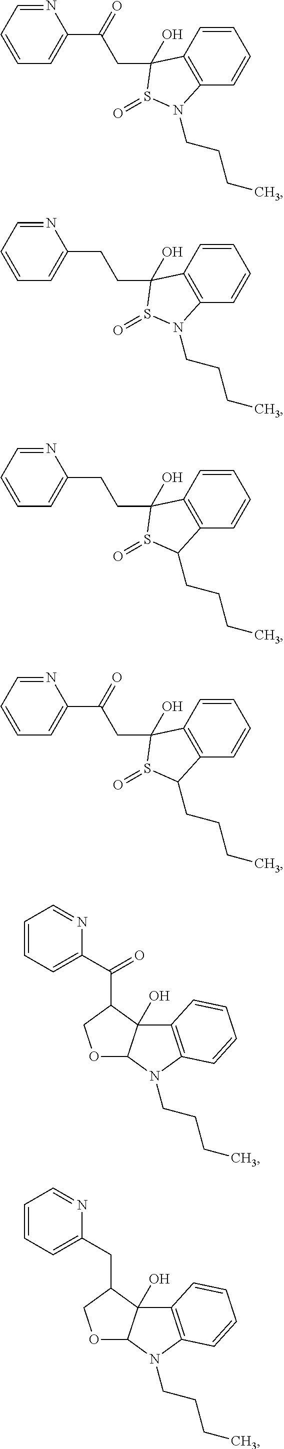 [SMILES: CCCCN1c2ccccc2C(O)(CC(=O)c2ccccn2)S1=O ; CCCCN1c2ccccc2C(O)(CCc2ccccn2)S1=O ; CCCCC1c2ccccc2C(O)(CCc2ccccn2)S1=O ; CCCCC1c2ccccc2C(O)(CC(=O)c2ccccn2)S1=O ; CCCCN1c2ccccc2C2(O)C(C(=O)c3ccccn3)COC12 ; CCCCN1c2ccccc2C2(O)C(Cc3ccccn3)COC12]

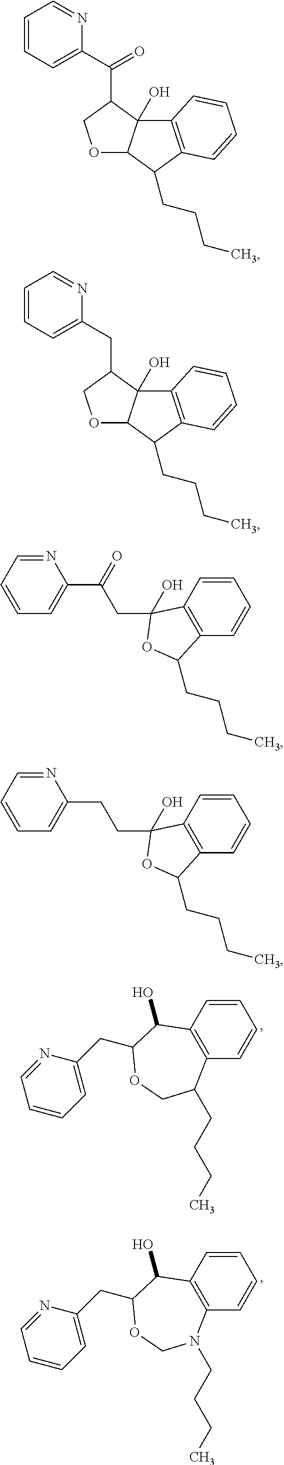

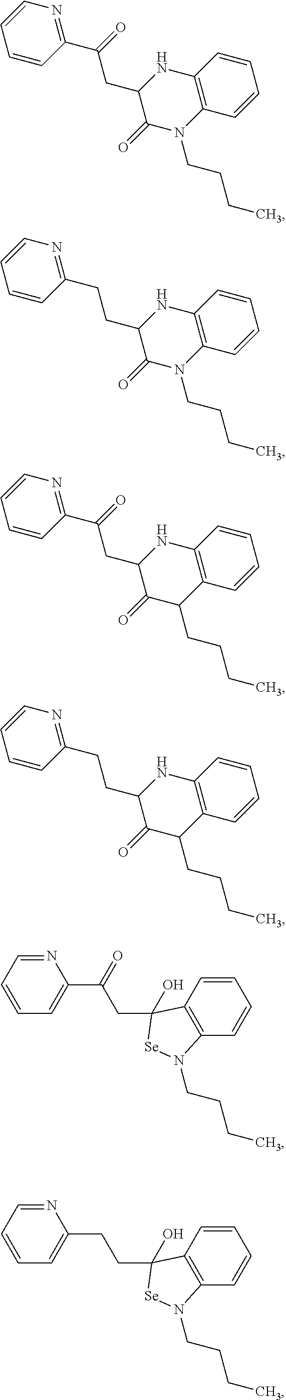 [SMILES: CCCCN1C(=O)C(CC(=O)c2ccccn2)Nc2ccccc21 ; CCCCN1C(=O)C(CCc2ccccn2)Nc2ccccc21 ; CCCCC1C(=O)C(CC(=O)c2ccccn2)Nc2ccccc21 ; CCCCC1C(=O)C(CCc2ccccn2)Nc2ccccc21 ; CCCCN1[Se]C(O)(CC(=O)c2ccccn2)c2ccccc21 ; CCCCN1[Se]C(O)(CCc2ccccn2)c2ccccc21]

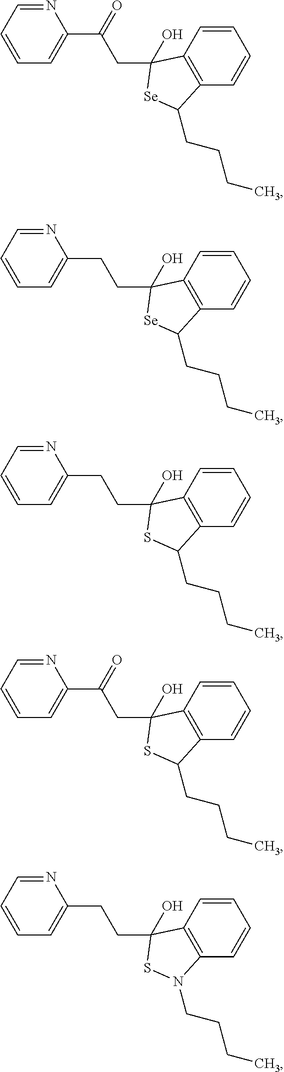 [SMILES: CCCCC1[Se]C(O)(CC(=O)c2ccccn2)c2ccccc21 ; CCCCC1[Se]C(O)(CCc2ccccn2)c2ccccc21 ; CCCCC1SC(O)(CCc2ccccn2)c2ccccc21 ; CCCCC1SC(O)(CC(=O)c2ccccn2)c2ccccc21 ; CCCCN1SC(O)(CCc2ccccn2)c2ccccc21]

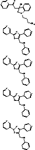

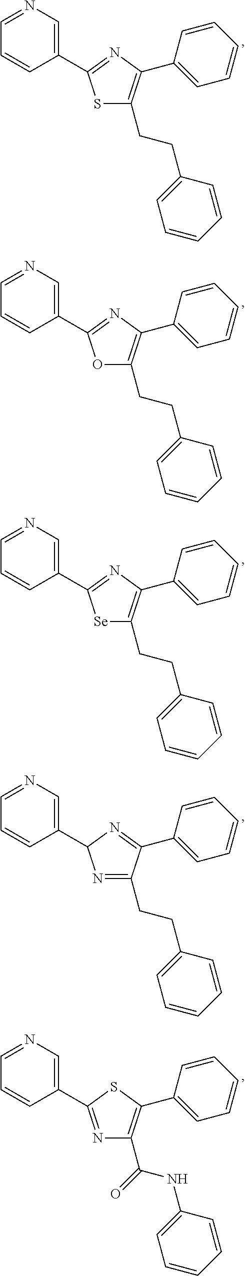

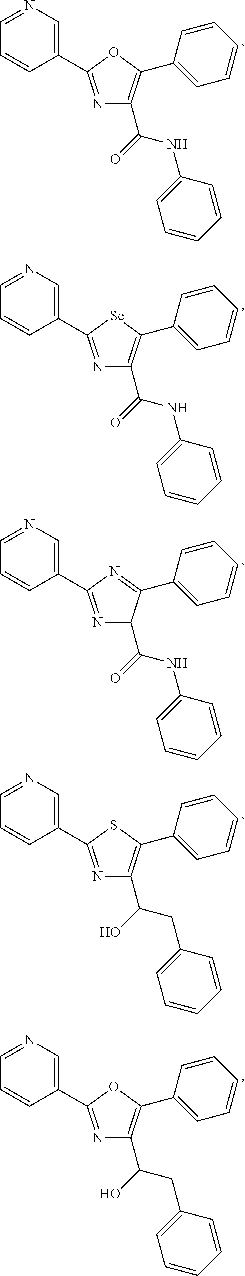

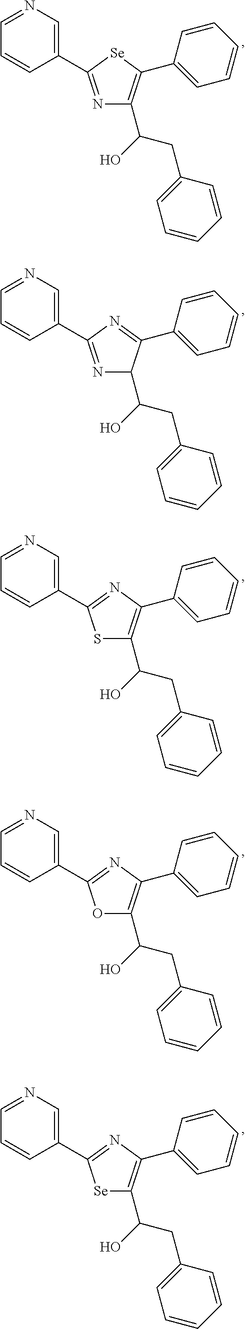 [SMILES: OC(Cc1ccccc1)c1nc(-c2cccnc2)[se]c1-c1ccccc1 ; OC(Cc1ccccc1)C1N=C(c2cccnc2)N=C1c1ccccc1 ; OC(Cc1ccccc1)c1sc(-c2cccnc2)nc1-c1ccccc1 ; OC(Cc1ccccc1)c1oc(-c2cccnc2)nc1-c1ccccc1 ; OC(Cc1ccccc1)c1[se]c(-c2cccnc2)nc1-c1ccccc1]

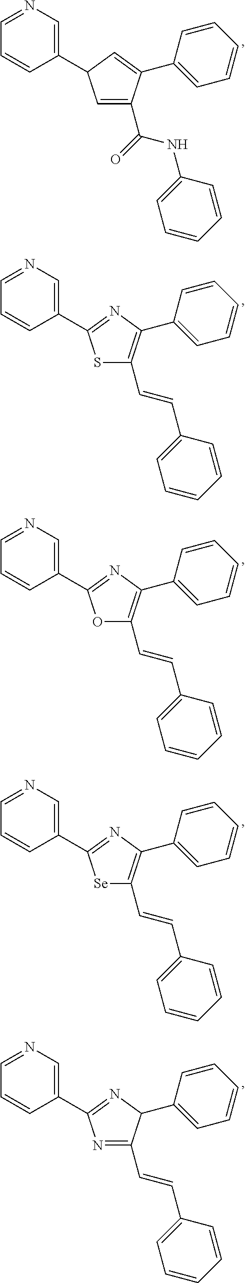

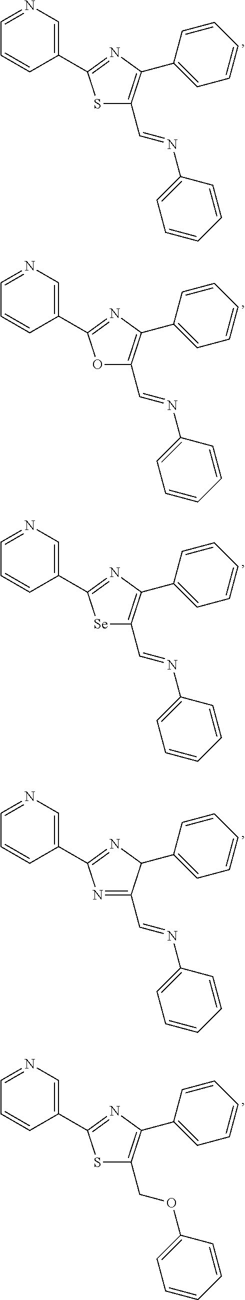

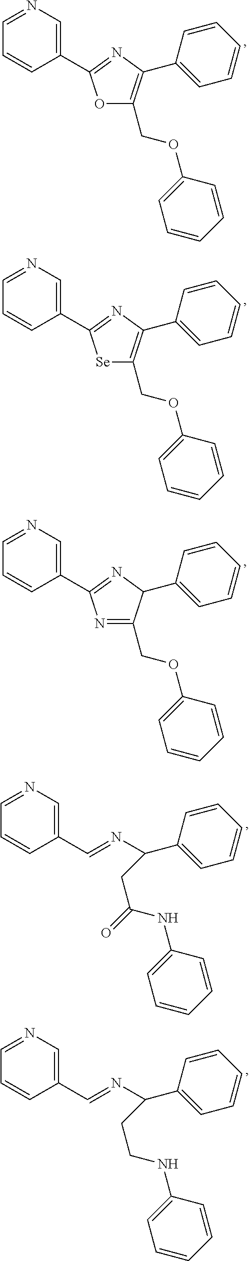

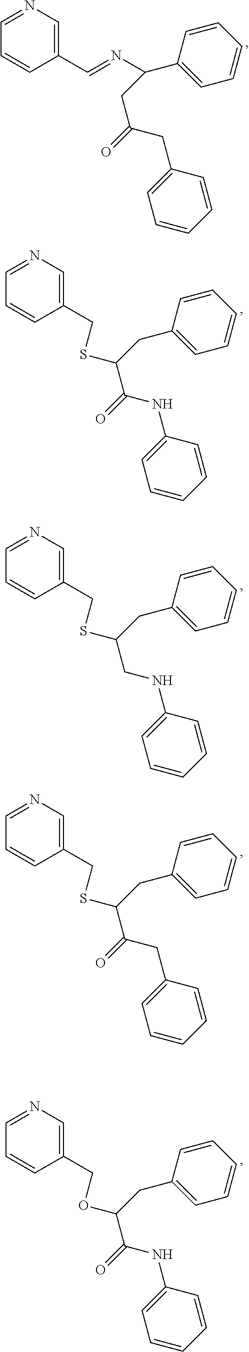 [SMILES: O=C(Cc1ccccc1)CC(/N=C/c1cccnc1)c1ccccc1 ; O=C(Nc1ccccc1)C(Cc1ccccc1)SCc1cccnc1 ; c1ccc(CC(CNc2ccccc2)SCc2cccnc2)cc1 ; O=C(Cc1ccccc1)C(Cc1ccccc1)SCc1cccnc1 ; O=C(Nc1ccccc1)C(Cc1ccccc1)OCc1cccnc1]

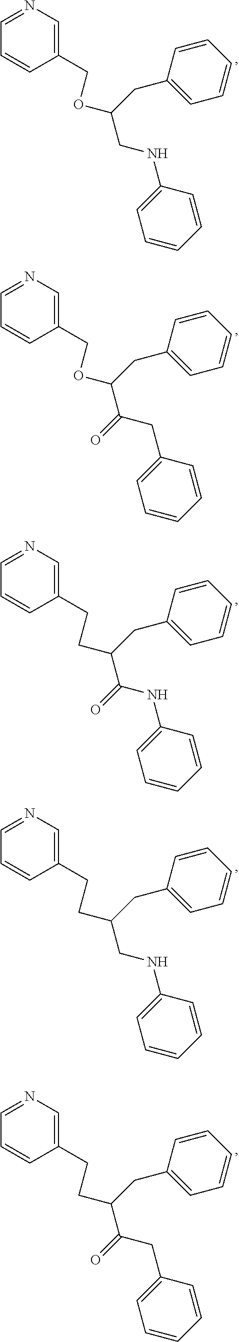 [SMILES: c1ccc(CC(CNc2ccccc2)OCc2cccnc2)cc1 ; O=C(Cc1ccccc1)C(Cc1ccccc1)OCc1cccnc1 ; O=C(Nc1ccccc1)C(CCc1cccnc1)Cc1ccccc1 ; c1ccc(CC(CCc2cccnc2)CNc2ccccc2)cc1 ; O=C(Cc1ccccc1)C(CCc1cccnc1)Cc1ccccc1]

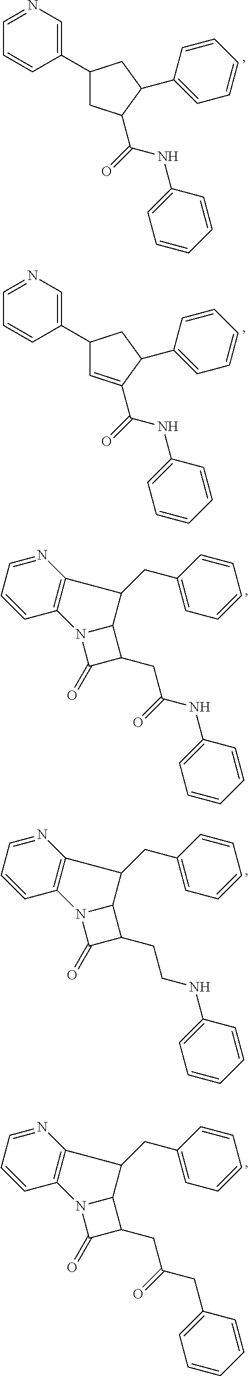

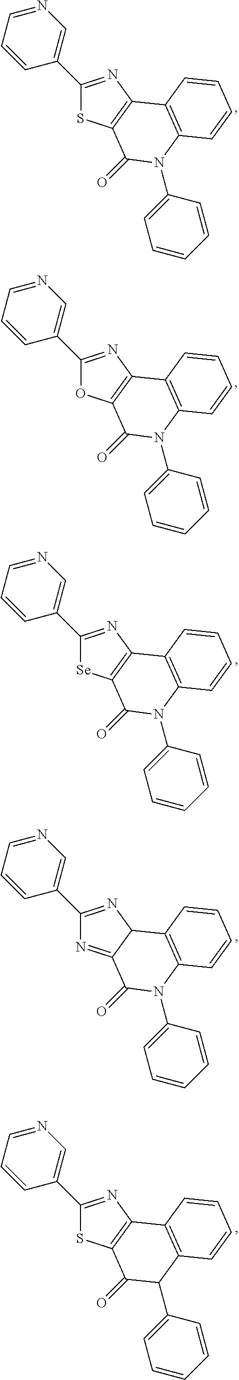 [SMILES: O=c1c2sc(-c3cccnc3)nc2c2ccccc2n1-c1ccccc1 ; O=c1c2oc(-c3cccnc3)nc2c2ccccc2n1-c1ccccc1 ; O=c1c2[se]c(-c3cccnc3)nc2c2ccccc2n1-c1ccccc1 ; O=C1C2=NC(c3cccnc3)=NC2c2ccccc2N1c1ccccc1 ; O=C1c2sc(-c3cccnc3)nc2-c2ccccc2C1c1ccccc1]

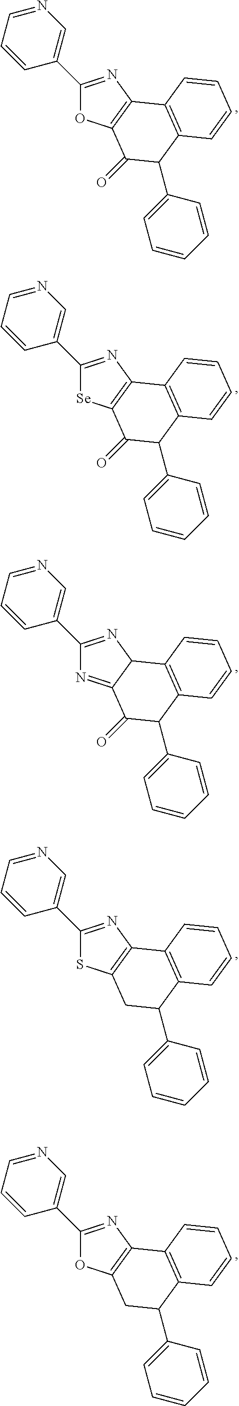 [SMILES: O=C1c2oc(-c3cccnc3)nc2-c2ccccc2C1c1ccccc1 ; O=C1c2[se]c(-c3cccnc3)nc2-c2ccccc2C1c1ccccc1 ; O=C1C2=NC(c3cccnc3)=NC2c2ccccc2C1c1ccccc1 ; c1ccc(C2Cc3sc(-c4cccnc4)nc3-c3ccccc32)cc1 ; c1ccc(C2Cc3oc(-c4cccnc4)nc3-c3ccccc32)cc1]

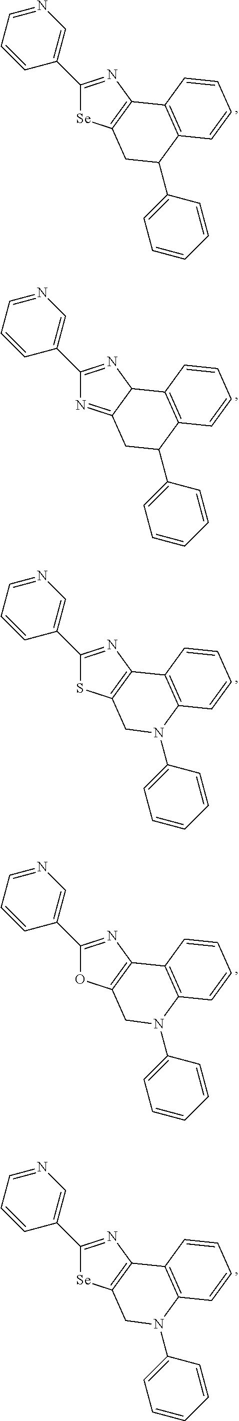 [SMILES: c1ccc(C2Cc3[se]c(-c4cccnc4)nc3-c3ccccc32)cc1 ; c1ccc(C2CC3=NC(c4cccnc4)=NC3c3ccccc32)cc1 ; c1ccc(N2Cc3sc(-c4cccnc4)nc3-c3ccccc32)cc1 ; c1ccc(N2Cc3oc(-c4cccnc4)nc3-c3ccccc32)cc1 ; c1ccc(N2Cc3[se]c(-c4cccnc4)nc3-c3ccccc32)cc1]

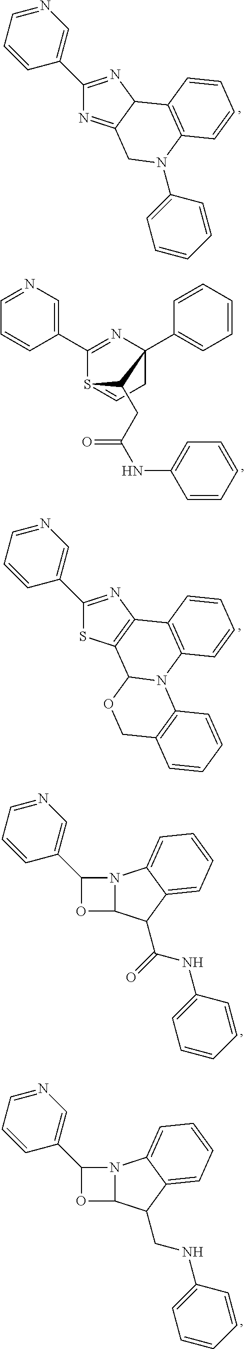

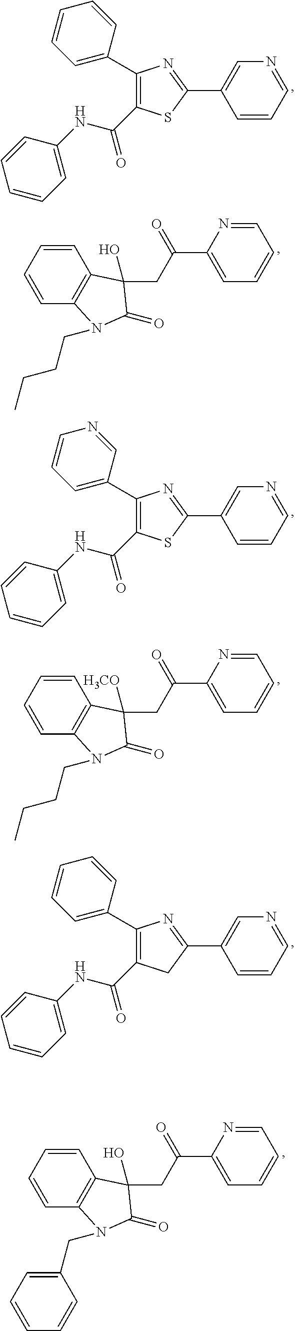

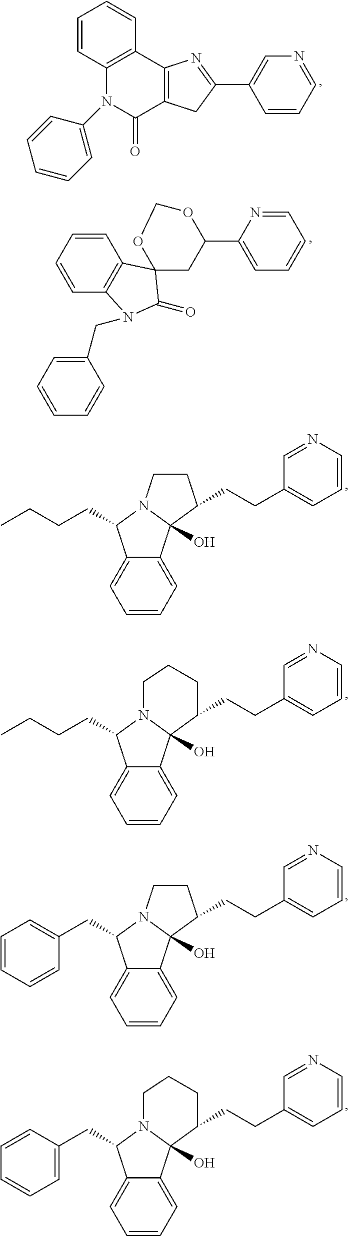 [SMILES: O=c1c2c(c3ccccc3n1-c1ccccc1)N=C(c1cccnc1)C2 ; O=C1N(Cc2ccccc2)c2ccccc2C12CC(c1ccccn1)OCO2 ; CCCC[C@H]1c2ccccc2[C@@]2(O)[C@@H](CCc3cccnc3)CCN12 ; CCCC[C@H]1c2ccccc2[C@@]2(O)[C@@H](CCc3cccnc3)CCCN12 ; O[C@@]12c3ccccc3[C@H](Cc3ccccc3)N1CC[C@@H]2CCc1cccnc1 ; O[C@@]12c3ccccc3[C@H](Cc3ccccc3)N1CCC[C@@H]2CCc1cccnc1]

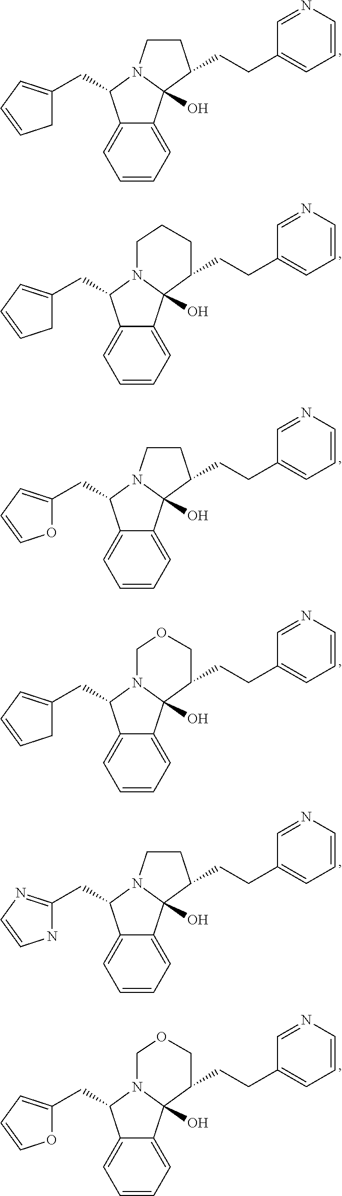

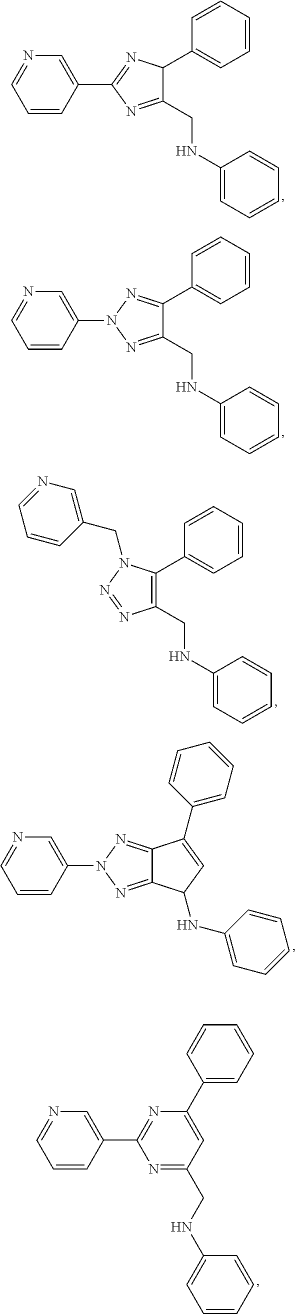

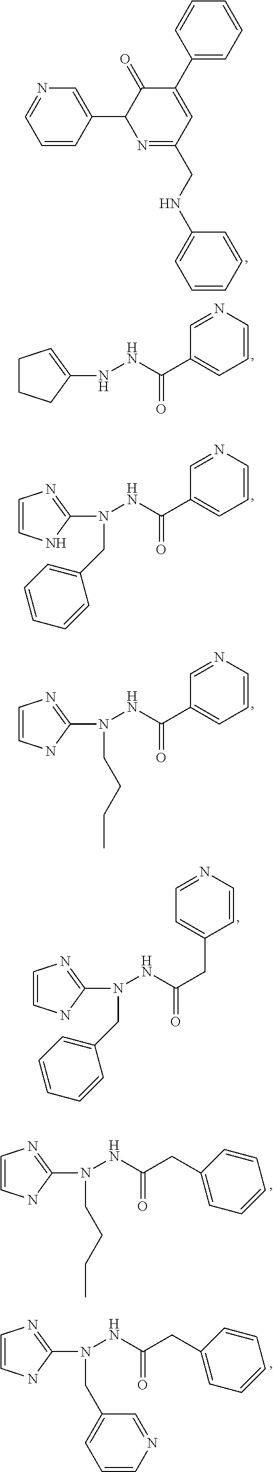

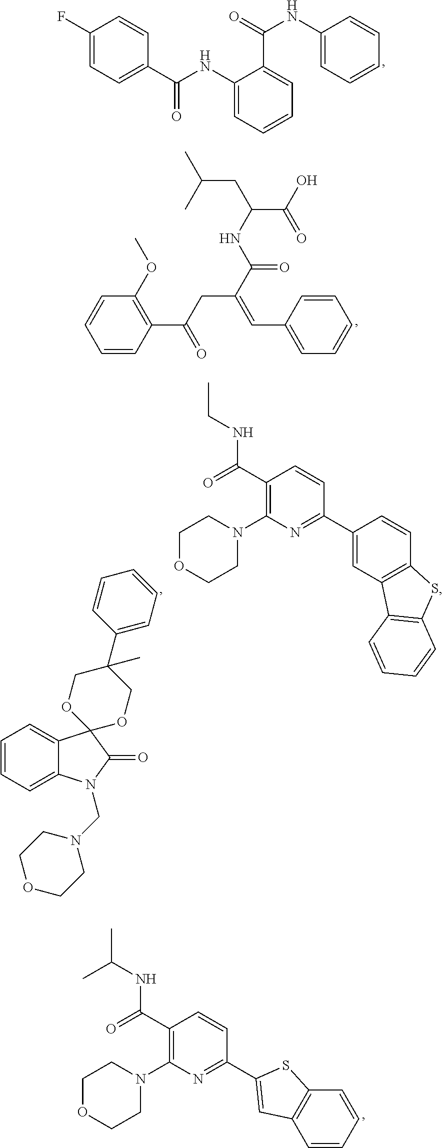

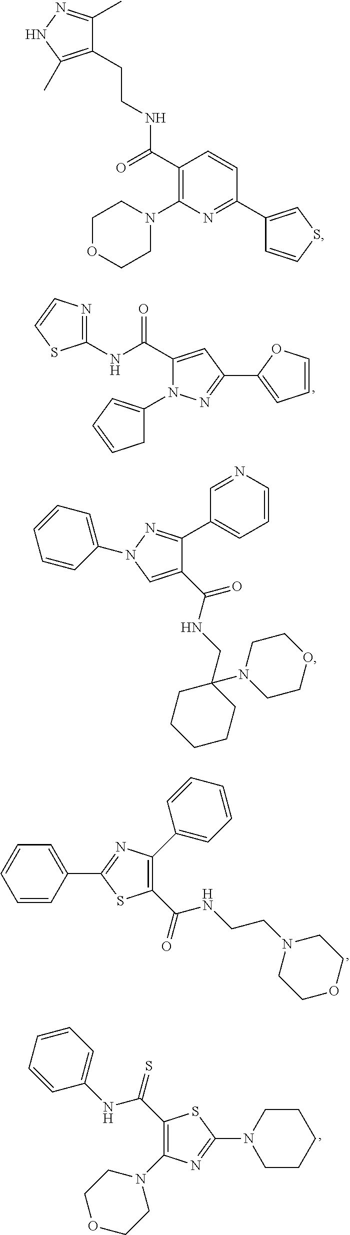

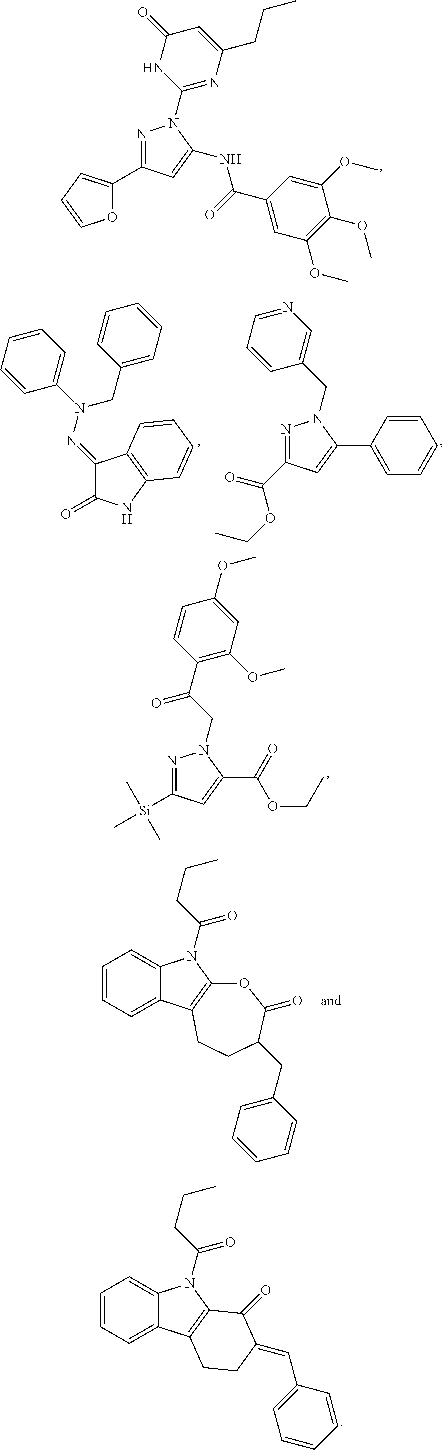

1. Isomers

When an asymmetric center is present in a compound of formulae I through IX, hereinafter referred to as the disclosed compounds, the compound may exist in the form of optical isomers (enantiomers). In one embodiment, the present invention comprises enantiomers and mixtures, including racemic mixtures of the compounds of formulae I through IX. In another embodiment, for compounds of formulae I through IX that contain more than one asymmetric center, the present invention comprises diastereomeric forms (individual diastereomers and mixtures thereof) of compounds. When a compound of formulae I through IX contains an alkenyl group or moiety, geometric isomers may arise.

2. Tautomeric Forms

The disclosed compositions and compounds comprise the tautomeric forms of compounds of formulae I through IX. Where structural isomers are interconvertible via a low energy barrier, tautomeric isomerism (‘tautomerism’) can occur. This can take the form of proton tautomerism in compounds of formula I containing, for example, an imino, keto, or oxime group, or so-called valence tautomerism in compounds which contain an aromatic moiety. It follows that a single compound may exhibit more than one type of isomerism. The various ratios of the tautomers in solid and liquid form is dependent on the various substituents on the molecule as well as the particular crystallization technique used to isolate a compound.

3. Salts

The disclosed compositions and compounds can be used in the form of salts derived from inorganic or organic acids. Depending on the particular compound, a salt of the compound can be advantageous due to one or more of the salt's physical properties, such as enhanced pharmaceutical stability in differing temperatures and humidities, or a desirable solubility in water or oil. In some instances, a salt of a compound also can be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of formulae I-V with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the disclosed compounds which are generally prepared by reacting the free base with a suitable organic or inorganic acid.

Suitable pharmaceutically acceptable acid addition salts of the disclosed compounds, when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclylic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

Furthermore, where the disclosed compounds carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibuytl, and diamyl sulfates), long chain halides (e.g., decyl, lauryl, myristyl, and stearyl chlorides, bromides, and iodides), arylalkyl halides (e.g., benzyl and phenethyl bromides), and others. In one embodiment, hemisalts of acids and bases may also be formed, for example, hemisulphate and hemicalcium salts. The disclosed compounds may exist in both unsolvated and solvated forms. A “solvate” as used herein is a nonaqueous solution or dispersoid in which there is a noncovalent or easily dispersible combination between solvent and solute, or dispersion means and disperse phase.

4. Prodrugs

Also disclosed are so-called “prodrugs” of the disclosed compounds. Thus, certain derivatives of the disclosed compounds which may have little or no pharmacological activity themselves can, when administered into or onto the body, be converted into the disclosed compounds having the desired activity, for example, by hydrolytic cleavage. Such derivatives are referred to as “prodrugs.” Further information on the use of prodrugs may be found in “Prodrugs as Novel Delivery Systems, Vol. 14, ACS Symposium Series (T Higuchi and W Stella) and “Bioreversible Carriers in Drug Design,” Pergamon Press, 1987 (ed. E B Roche, American Pharmaceutical Association). Prodrugs as disclosed herein can, for example, be produced by replacing appropriate functionalities present in the compounds of any of formulae I through IX with certain moieties known to those skilled in the art as “pro-moieties” as described, for example, in “Design of Prodrugs” by H Bundgaard (Elsevier, 1985).

5. Isotopes

Also disclosed are isotopically labelled compounds, which are identical to those recited in formulae I through IX, but for the fact that one or more atoms are replaced by an atom having an atomic mass or mass number different from the atomic mass or mass number usually found in nature.

Examples of isotopes that can be incorporated into disclosed compounds include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Disclosed compounds, prodrugs thereof, and pharmaceutically acceptable salts of said compounds or of said prodrugs which contain the aforementioned isotopes and/or other isotopes of other atoms are contemplated. Certain isotopically labelled disclosed compounds, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula I through IX and prodrugs thereof can generally be prepared by carrying out the procedures disclosed in the Schemes and/or in the Examples and Preparations below, by substituting a readily available isotopically labelled reagent for a non-isotopically labelled reagent.

6. General Synthetic Schemes

The compounds of the formulae I through IX may be prepared by the methods described below, together with synthetic methods known in the art of organic chemistry, or modifications and derivatisations that are familiar to those of ordinary skill in the art. The starting materials used herein are commercially available or may be prepared by routine methods known in the art (such as those methods disclosed in standard reference books such as the COMPENDIUM OF ORGANIC SYNTHETIC METHODS, Vol. I-VI (published by Wiley-Interscience)). Preferred methods include, but are not limited to, those described below. During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991, and T. W. Greene and P. G. M. Wuts, Protective Groups in Organic Chemistry, John Wiley & Sons, 1999, which are hereby incorporated by reference. Compounds of formulae I through IX, or their pharmaceutically acceptable salts, can be prepared according to the reaction Schemes discussed herein. Unless otherwise indicated, the substituents in the Schemes are defined as herein. Isolation and purification of the products is accomplished by standard procedures, which are known to a chemist of ordinary skill. The following schemes are exemplary of the processes for making compounds of formulae I through IX.

The formula I compounds encompass a number of structural classes and each class has different syntheses. Thus, the syntheses of compounds within the structural class of formula I are embodied in the syntheses of formulas II through IX as described below.

Scheme I illustrates a method for the preparation of compounds of formula II:

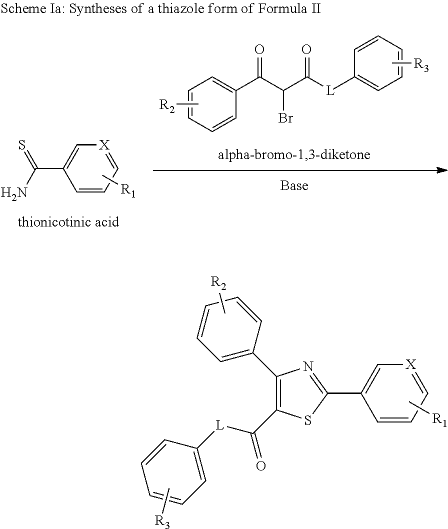

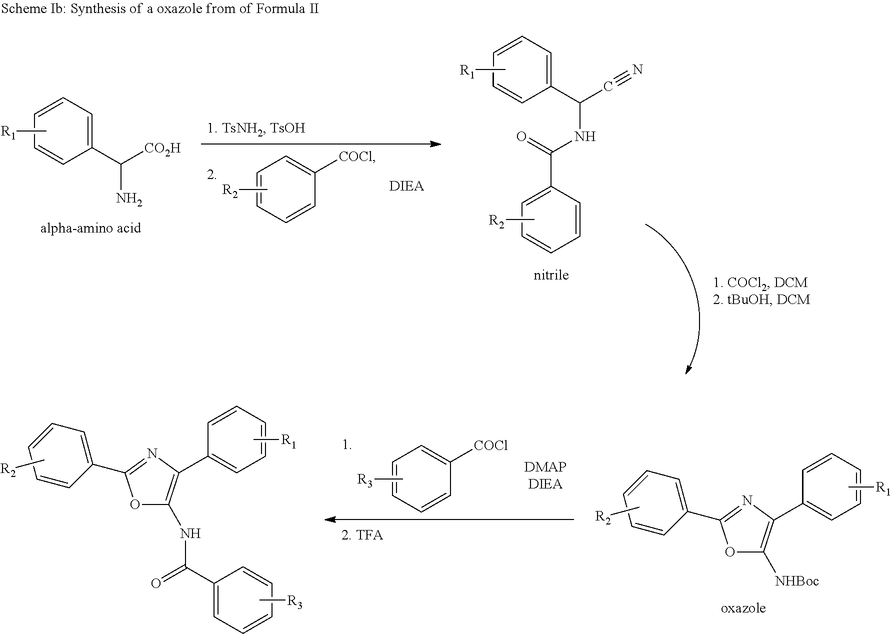

Referring to scheme I, a compound of formula II can be synthesized as shown below:

Scheme Ia. A substituted thionicotinic acid (or thiobenzoic acid where X=CG) is dissolved in 10% aqueous ethanol solution containing 0.1 M KOH and an ethanolic solution of an alpha-bromo-1,3-diketone is added dropwise with stirring over one hour and then the solution is stirred overnight to give the condensed thiazole. The solution is then partly evaporated and triturated with water causing the product to precipitate out of solution. The product is then recrystallized from ethyl acetate.

Scheme Ib. An alpha amino acid is dissolved in refluxing 1,4-dioxane containing 0.1% toluenesulfonic acid (TsOH) to which is added dropwise a dioxane solution of toluenesulfonolamide (TsNH₂) over 1 h. The solution is refluxed overnight and then cooled to room temperature. A solution of and acid chloride in dry THF containing 1% DIEA is added dropwise with cooling on ice over 1 hr. The resulting solution is washed several times with brine, and evaporated to give an oil that is then purified by silica gel chromatography to give the purified nitrile. The nitrile is dissolved in dichloromethane (DCM) to which is added a solution of phosgene in benzene. After 5 min at room temperature, a solution of tBuOH in DCM is added and after 5 min the solution is washed with brine, evaporated and the resulting oil is purified by silica gel chromatography. The Boc-protected oxazole is then dissolved in dry THF with 1% DIEA and trace DMAP, to which is added an acid chloride. After stirring for 2 h at room temperature, TFA is added to a final concentration of 20% and the solution is stirred for 18 h. After washing with brine and evaporation of the solution, the resulting oil is purified by silica gel chromatography to give the final oxazole.

Scheme II illustrates a method for the preparation of compounds of formula III:

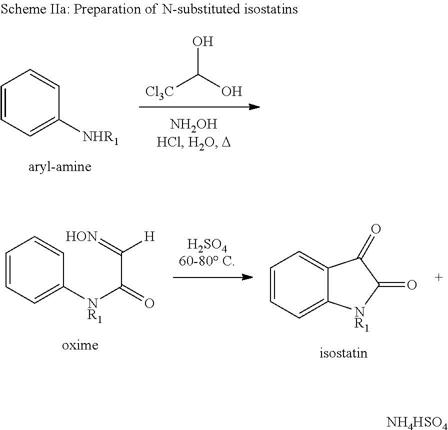

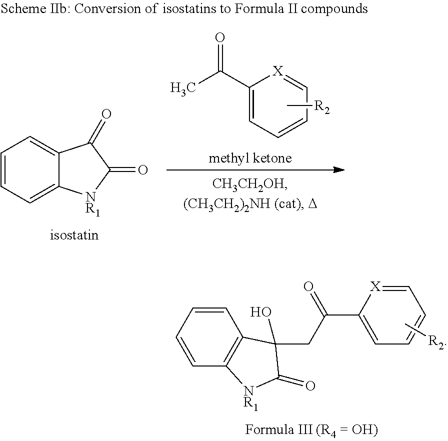

Referring to scheme II, a compound of formula III can be synthesized as shown below:

N-substituted aryl amines are condensed with trichloroacetaldehyde hydrate in an acidic solution of ethanol:water (50:50) containing an excess of hydroxylamine. The solution is refluxed for 1-2 h to give the oxime condensation product that is extracted with ether from the aqueous liquor remaining after evaporation of the ethanol. Upon evaporation of the dried ether extract, the crude oxime is dissolved in 20% aqueous sulfuric acid and heated overnight at 60-80° C. to five the cyclized isostatin. After extraction of the aqueous solution with ether and evaporation, the isostatin is isolated and purified by either silica gel chromatography or distillation in vacuo.

Scheme III illustrates a method for the preparation of compounds of formula IV:

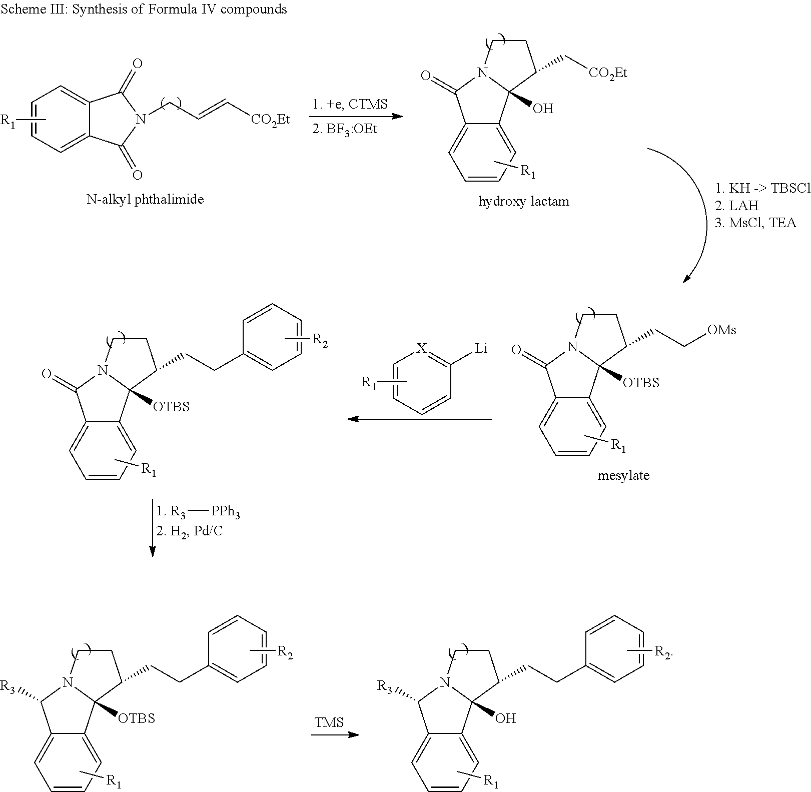

Scheme IV illustrates a method for the preparation of compounds of formula V:

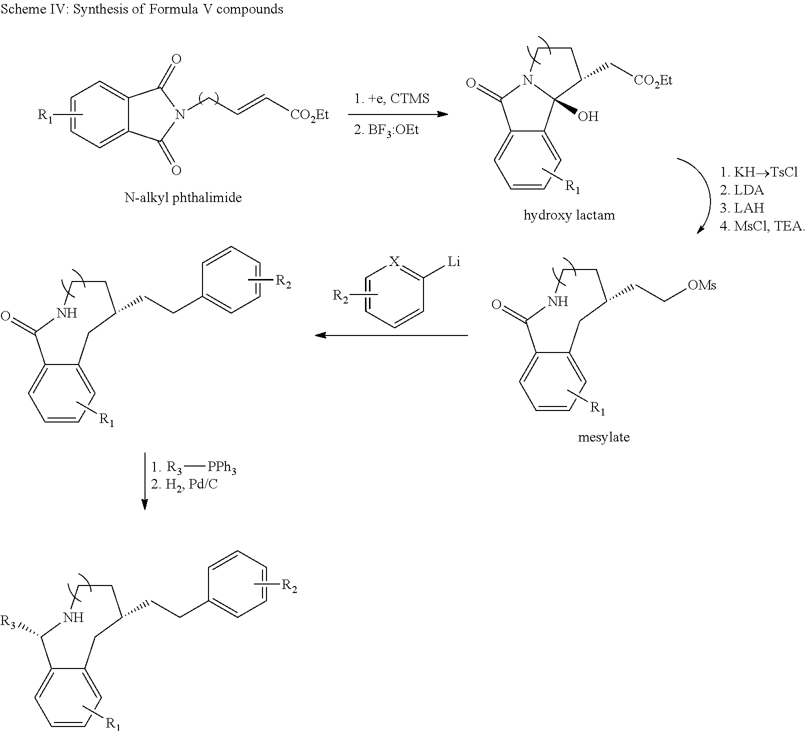

Referring to schemes III and IV, a compound of formula IV and formula V can be synthesized as shown below:

An N-alkyl phthalimide is electroductively coupled to give a hydroxy lactam. The phthalimide is dissolved in a solution of 0.3 M Et₄NOTs in DMF in a coulometric cell and a current of 100 mA is applied for 30 min after which the solution is dissolved in water and frozen. After lyophilization, the residue is dissolved in 50:50 water:ether and the ether layer is removed. Washed with brine and evaporated to give the hydroxy lactam. The hydroxy group is then protected with TBS after which the ester is reduced to the alcohol with LAH. The alcohol is converted to the mesylate with mesyl chloride and the mesylate is purified by silica gel chromatography. Alternatively, as shown in scheme IV, the alcohol is converted to a tosylate, which is then eliminated with LDA causing ring opening of the lactam after which the ester is reduced and converted to the ring-opened mesylate. Addition of an aryl-lithium complex in THF to the mesylate in THF at −78° C. and then bringing to room temperature gives an aryl substituted lactam that is then treated with a triphenylphosphonium iodide salt followed by hydrogenation to give the penultimate product. After deprotection with TMS, the final product is obtained via silica gel chromatography.

Scheme V illustrates a method for the preparation of compounds of formulae VI to IX:

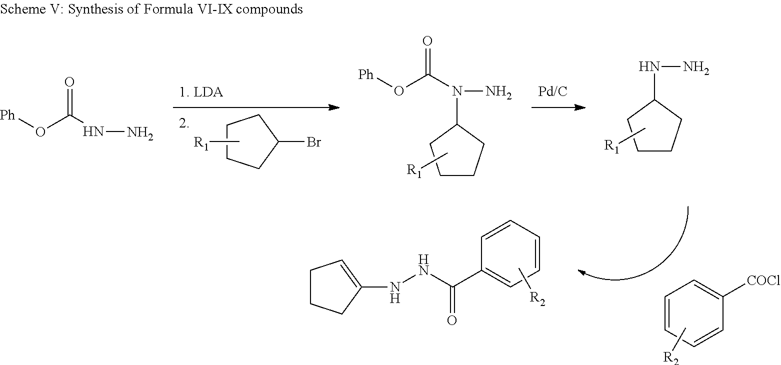

Referring to scheme V, a compound of formulae VI-IX can be synthesized as shown below:

Carboxybenzamate hydrazine is dissolved in dry THF and a solution of LDA in THF is added dropwise over 15 min followed by addition of an alkyl halide (or ketone for the alkenyl substitution). After stirring for 1 h, the solution is washed with brine, evaporated, and the resulting oil is dissolved in ethanol and hydrogenated overnight at 15 psi using Pd/C catalyst. After evaporation, the crude oil is dissolved in dry DCM and then treated with an acid chloride for 2 h, followed by evaporation, and subsequent purification by silica gel chromoatography.

### C. METHODS

The compounds of formulae I through IX are useful for the prevention and/or treatment of degenerative diseases. Accordingly, in one embodiment, disclosed are methods of preventing and/or treating degenerative disease in a subject, comprising administering to said subject a therapeutically effective amount of a compound of formulae I through IX, or a pharmaceutically acceptable salt thereof. In another embodiment, the method of preventing and/or treating degenerative disease in a subject is a method, wherein said degenerative disease is associated with mitochondrial damage and/or dysfunction. In another embodiment, the method of preventing and/or treating degenerative disease in a subject is a method, wherein said compound of formulae I through IX, or a pharmaceutically acceptable salt thereof, is effective in maintaining, modulating or improving mitochondrial metabolic function.

In another embodiment, the method of preventing and/or treating degenerative disease in a subject is a method, wherein said degenerative disease is selected from the group consisting of retinal degenerative disease, Alzheimer's disease, Parkinson's diseases, Friedreich's ataxia, Huntington's disease, heart failure, myocardial fraction, atherosclerosis, stroke, renal dysfunction, type II diabetes, diabetes mellitus and deafness (DAD), Leber's hereditary optic neuropathy (LHON), Leigh syndrome, subacute sclerosing encephalopathy, Neuropathy, ataxia, retinitis pigmentosa, and ptosis (NARP), Myoneurogenic gastrointestinal encephalopathy (MNGIE), Myoclonic Epilepsy with Ragged Red Fibers (MERRF), and Mitochondrial myopathy, encephalomyopathy, lactic acidosis, stroke-like symptoms (MELAS).

In another embodiment, the method of preventing and/or treating degenerative disease in a subject is a method, wherein said degenerative disease is retinal degenerative disease which is selected from the group consisting of age-related mascular degeneration, Stargardt's disease, glaucoma, retinitis pignmentosa, and optic nerve degeneration. In another embodiment, the method of preventing and/or treating degenerative disease in a subject is a method, wherein said retinal degenerative disease is retinitis pigmnentosa. In another embodiment, the method of preventing and/or treating degenerative disease in a subject is a method, wherein said compound of formulae I through IX, or a pharmaceutically acceptable salt thereof, is effective in inhibiting and/or reducing the progression of retinal degeneration in retinitis pigmentosa. In another embodiment, the method of preventing and/or treating degenerative disease in a subject is a method, wherein said compound of formulae I through IX, or a pharmaceutically acceptable salt thereof, is effective in protecting retinal cells and tissues from calcium induced injury, oxidative stress induced injury, or apoptotic cell death.

Typically, a disclosed compound is administered in an amount effective to treat or prevent a condition as described herein. The disclosed compounds can be administered by any suitable route in the form of a pharmaceutical composition adapted to such a route, and in a dose effective for the treatment or prevention intended. Therapeutically effective doses of the compounds required to treat or prevent the progress of the medical condition are readily ascertained by one of ordinary skill in the art using preclinical and clinical approaches familiar to the medicinal arts. The disclosed compounds can be administered orally. Oral administration can involve swallowing, so that the compound enters the gastrointestinal tract, or buccal or sublingual administration can be employed by which the compound enters the blood stream directly from the mouth.

The disclosed compounds can also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques. The disclosed compounds can also be administered topically to the skin or mucosa, that is, dermally or transdermally. The disclosed compounds can also be administered intranasally or by inhalation. The disclosed compounds can be administered rectally, vaginally, or directly to the eye or ear.

In another embodiment, the method of preventing and/or treating degenerative disease in a subject is a method, wherein said compound of formulae I through IX, or a pharmaceutically acceptable salt thereof, is administered by one or more routes selected from a group consisting of rectal, buccal, sublingual, intravenous, subcutaneous, intradermal, transdermal, intraperitoneal, oral, parenteral and topical administration.

In another embodiment, the method of preventing and/or treating degenerative disease in a subject is a method, wherein said topical administration is via a carrier vehicle selected from the group consisting of drops of liquid, liquid washes, gels, ointments, sprays and liposomes. Alternatively, disclosed are methods, wherein said topical administration is via drops of liquid, liquid washes, gels, ointments, sprays or liposomes. In another embodiment, the method of preventing and/or treating degenerative disease in a subject is a method, wherein said topical administration comprises infusion of said compound of formulae I through IX, or a pharmaceutically acceptable salt thereof, to an ocular surface via a device selected from the group consisting of a pump-catheter system, a continuous or selective release device and a contact lens.

In another embodiment, the method of preventing and/or treating degenerative disease in a subject is a method, wherein said administration is administration of a liquid/liquid suspension of said compound of formulae I through IX, or a pharmaceutically acceptable salt thereof, via nose drops or nasal spray, or administration of a nebulized liquid to oral or nasopharyngeal airways of said subject. In another embodiment, the method of preventing and/or treating degenerative disease in a subject is a method, wherein said administration is accomplished by administering an oral form of said compound of formulae I through IX, or a pharmaceutically acceptable salt thereof. In another embodiment, the method of preventing and/or treating degenerative disease in a subject is a method, wherein said administration is administration of an injectable form of said compound of formulae I through IX, or a pharmaceutically acceptable salt thereof.

In another embodiment, the method of preventing and/or treating degenerative disease in a subject is a method, wherein said administration is administration of a suppository form of said compound of formulae I through IX, or a pharmaceutically acceptable salt thereof. In another embodiment, the method of preventing and/or treating degenerative disease in a subject is a method, wherein said administration is administration of a of an intra-operative instillation of a gel, cream, powder, foam, crystals, liposomes, spray or liquid suspension form of said compound of formulae I through IX, or a pharmaceutically acceptable salt thereof. In another embodiment, the method of preventing and/or treating degenerative disease in a subject is a method, wherein said administration is administration of said compound of formulae I through IX, or a pharmaceutically acceptable salt thereof, in a form of a transdermal patch or a transdermal pad.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.001 mg to about 100 mg per kilogram of body weight per day are useful in the treatment or prevention of the above-indicated conditions. Other effective dosages regimens of a disclosed compounds (administered in single or divided doses) include but not limited to: from about 0.01 to about 100 mg/kg/day, from about 0.1 to about 50 mg/kg/day, from about 0.5 to about 30 mg/kg/day, from about 0.01 to about 10 mg/kg/day, and from about 0.1 to about 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration, the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or from about 1 mg to about 100 mg of active ingredient. Intravenously, doses may range from about 0.1 to about 10 mg/kg/minute during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

In another embodiment, the method of preventing and/or treating degenerative disease in a subject is a method, wherein said compound of formulae I through IX, or a pharmaceutically acceptable salt thereof, is administered in an amount of about 0.001 to about 100 mg/kg body weight on days of administration. In another embodiment, the method of preventing and/or treating degenerative disease in a subject is a method, wherein said compound of formulae I through IX, or a pharmaceutically acceptable salt thereof, is administered in an amount of about 0.1 to about 100 mg/kg body weight on days of administration. In another embodiment, the method of preventing and/or treating degenerative disease in a subject is a method, wherein said compound of formulae I through IX, or a pharmaceutically acceptable salt thereof, is administered in an amount of about 1 to about 100 mg/kg body weight on days of administration. In another embodiment, the method of preventing and/or treating degenerative disease in a subject is a method, wherein said compound of formulae I through IX, or a pharmaceutically acceptable salt thereof, is administered in an amount of about 1 to about 50 mg/kg body weight on days of administration. In another embodiment, the method of preventing and/or treating degenerative disease in a subject is a method, wherein said compound of formulae I through IX, or a pharmaceutically acceptable salt thereof, is administered in an amount of about 10 to about 50 mg/kg body weight on days of administration.

In another embodiment, the method of preventing and/or treating degenerative disease in a subject is a method further comprises one or more antidegenerative agents. In another embodiment, the method of preventing and/or treating degenerative disease in a subject is a method, wherein said compound of formulae I through IX, or a pharmaceutically acceptable salt thereof, and said one or more antidegenerative agents are administered in separate formulation. In another embodiment, the method of preventing and/or treating degenerative disease in a subject is a method, wherein said compound of formulae I through IX, or a pharmaceutically acceptable salt thereof, and said one or more antidegenerative agents are administered in the same formulation.

In another embodiment, the method of preventing and/or treating degenerative disease in a subject is a method, wherein said compound of formulae I through IX, or a pharmaceutically acceptable salt thereof, and said one or more antidegenerative agents are administered concurrently or sequentially. In another embodiment, the method of preventing and/or treating degenerative disease in a subject is a method, wherein said compound of formulae I through IX, or a pharmaceutically acceptable salt thereof, and said one or more antidegenerative agents are administered by the same or different routes.

In another embodiment, the method of preventing and/or treating degenerative disease in a subject is a method, wherein said compound of formulae I through IX, or a pharmaceutically acceptable salt thereof, and said one or more antidegenerative agents produce synergistic effect in preventing and/or treating degenerative disease. In another embodiment, the method of preventing and/or treating degenerative disease in a subject is a method, wherein said degenerative disease is retinal degenerative disease. In another embodiment, the method of preventing and/or treating degenerative disease in a subject is a method, wherein said retinal degenerative disease is retinitis pigmnentosa. In another embodiment, the method of preventing and/or treating degenerative disease in a subject is a method, wherein said degenerative disease is insensitive, resistant or refractory to treatment with said compound of formulae I through IX, or a pharmaceutically acceptable salt thereof, or said one or more antidegenerative agents administered as a single agent.

In another embodiment, the method of preventing and/or treating degenerative disease in a subject is a method, wherein said compound of formulae I through IX, or a pharmaceutically acceptable salt thereof, and said one or more antidegenerative agents are each administered in an amount of from 1/100 to less than ½ of their normal individual therapeutic doses. In another embodiment, the method of preventing and/or treating degenerative disease in a subject is a method, wherein said compound of formulae I through IX, or a pharmaceutically acceptable salt thereof, and said one or more antidegenerative agents are each administered in an amount of from 1/10 to less than ¼ of their normal individual therapeutic doses.

In another embodiment, the method of preventing and/or treating degenerative disease in a subject is a method, wherein said one or more antidegenerative agents are selected from the group consisting of cyclosporin, NIM811, minocycline, macugen, lucentis, avastin, SIRt activator such as SRL₂₁₀₄, SRL₂₃₇₈, SRL₅₀₁, quercetin, resveratrol and the like, anti-interferon agent such as MEDI-545 and the like, and anti-TNF agent such as etanercept and the like. In another embodiment, the method of preventing and/or treating degenerative disease in a subject is a method, wherein the subject is a mammal.

### D. PHARMACEUTICAL COMPOSITIONS

For the treatment and/or prevention of the conditions referred to above, the disclosed compounds can be administered as compound per se. Alternatively, pharmaceutically acceptable salts are suitable for medical applications because of their greater aqueous solubility relative to the parent compound. In one embodiment, the present invention provides a pharmaceutical composition for preventing and/or treating degenerative disease in a subject comprising a therapeutically effective amount of a compound of formulae I through IX, or a pharmaceutically acceptable salt thereof.

In another embodiment, the pharmaceutical composition is a composition, wherein said compound of formulae I through IX, or a pharmaceutically acceptable salt thereof, is effective in maintaining, modulating or improving mitochondrial metabolic function. In another embodiment, the pharmaceutical composition is a composition, wherein said compound of formulae I through IX, or a pharmaceutically acceptable salt thereof, is effective in inhibiting and/or reducing the progression of retinal degeneration in retinitis pigmnentosa. In another embodiment, the pharmaceutical composition is a composition, wherein said compound of formulae I through IX, or a pharmaceutically acceptable salt thereof, is effective in protecting retinal cells and tissues from calcium induced injury, oxidative stress induced injury, or apoptotic cell death.

In another embodiment, the pharmaceutical composition further comprises one or more antidegenerative agents. In another embodiment, the pharmaceutical composition is a composition, wherein said compound of formulae I through IX, or a pharmaceutically acceptable salt thereof, and one or more antidegenerative agents produces synergistic effect in preventing and/or treating degenerative disease in a subject.

In another embodiment, the pharmaceutical composition is a composition, wherein said degenerative disease is retinal degenerative disease. In another embodiment, the pharmaceutical composition is a composition, wherein said retinal degenerative disease is retinitis pigmnentosa.

In another embodiment, the pharmaceutical composition is a composition, wherein said compound of formulae I through IX, or a pharmaceutically acceptable salt thereof, to said one or more antidegenerative agents ranges from about 1:100 to about 100:1. In another embodiment, the pharmaceutical composition is a composition, wherein said compound of formulae I through IX, or a pharmaceutically acceptable salt thereof, to said one or more antidegenerative agents ranges from about 1:50 to about 50:1. In another embodiment, the pharmaceutical composition is a composition, wherein said compound of formulae I through IX, or a pharmaceutically acceptable salt thereof, to said one or more antidegenerative agents ranges from about 1:10 to about 10:1. In another embodiment, the pharmaceutical composition is a composition, wherein said compound of formulae I through IX, or a pharmaceutically acceptable salt thereof, to said one or more antidegenerative agents ranges from about 1:5 to about 5:1.

In another embodiment, the pharmaceutical composition is a composition, wherein said one or more antidegenerative agents are selected from the group consisting of cyclosporin, NIM811, minocycline, macugen, lucentis, avastin, SIRt activator such as SRL₂₁₀₄, SRL₂₃₇₈, SRL₅₀₁, quercetin, resveratrol and the like, anti-interferon agent such as MEDI-545 and the like, and anti-TNF agent such as etanercept and the like. In another embodiment, the pharmaceutical composition is a composition, wherein the subject is a mammal.

In another embodiment, the pharmaceutical compositions as described above, further comprise a pharmaceutically-acceptable carrier. By “pharmaceutically acceptable”, it is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. The carrier can be a solid, a liquid, or both, and may be formulated with the compound as a unit-dose composition, for example, a tablet, which can contain from 0.05% to 95% by weight of the active compounds. A disclosed compounds may be coupled with suitable polymers as targetable drug carriers. Other pharmacologically active substances can also be present.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered. Pharmaceutical carriers are known to those skilled in the art. These most typically would be standard carriers for administration of drugs to humans, including solutions such as sterile water, saline, and buffered solutions at physiological pH. In another embodiment, the pharmaceutical compositions as described above, further comprise thickeners, diluents, buffers, preservatives, surface active agents and the like.

The disclosed compounds may be administered by any suitable route, preferably in the form of a pharmaceutical composition adapted to such a route, and in a dose effective for the treatment or prevention intended. The active compounds and compositions, for example, may be administered orally, rectally, parenterally, or topically.

Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sublingual, such as, for example, a lozenge. In such solid dosage forms, the compounds of formulae I through IV are ordinarily combined with one or more adjuvants. Such capsules or tablets may contain a controlled-release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the present invention comprises a parenteral dose form. “Parenteral administration” includes, for example, subcutaneous injections, intravenous injections, intraperitoneally, intramuscular injections, intrasternal injections, and infusion. Injectable preparations (e.g., sterile injectable aqueous or oleaginous suspensions) may be formulated according to the known art using suitable dispersing, wetting agents, and/or suspending agents.

In another embodiment, the present invention comprises a topical dose form. “Topical administration” includes, for example, transdermal administration, such as via transdermal patches or iontophoresis devices, intraocular administration, or intranasal or inhalation administration-Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Formulations suitable for topical administration to the eye include, for example, eye drops wherein the compound of this invention is dissolved or suspended in suitable carrier. A typical formulation suitable for ocular or aural administration may be in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active disclosed compounds are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3 Ed.), American Pharmaceutical Association, Washington, 1999.

The disclosed compounds can be used, alone or in combination with other therapeutic agents, in the treatment or prevention of various conditions or disease states. The compound(s) of the present invention and other therapeutic agent(s) may be administered simultaneously (either in the same dosage form or in separate dosage forms) or sequentially. An exemplary therapeutic agent may be, for example, an antidegenerative agent.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration. The phrases “concurrent administration,” “co-administration,” “simultaneous administration,” and “administered simultaneously” mean that the compounds are administered in combination.

### E. KITS

The present invention further comprises kits that are suitable for use in performing the methods of treatment or prevention described above. In one embodiment, the kit contains a first dosage form comprising one or more of the disclosed compounds and a container for the dosage, in quantities sufficient to carry out the methods of the present invention. In another embodiment, the kit of the present invention comprises one or more disclosed compounds. In another embodiment, the kit of the present invention comprises one or more disclosed compounds, and one or more other therapeutic agents. An exemplary therapeutic agent may be, for example, an antidegenerative agent.

### F. Definitions

1. A, an the

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

2. Weight %

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

3. Antidegenerative Agent

An antidegenerative agent or like term is any molecule or composition in which the molecule or composition reduces or inhibits the degeneration of mitochondria.

4. Binding Affinity

The term binding affinity as used herein can be defined as two molecules interacting with a kd of at least 10⁻³, 10⁻⁴, 10⁻⁵, 10⁻⁶, 10⁻⁷, 10⁻⁸, or 10⁻⁹ M or tighter binding.

5. Cell

The term “cell” as used herein also refers to individual cells, cell lines, or cultures derived from such cells. A “culture” refers to a composition comprising isolated cells of the same or a different type. The term co-culture is used to designate when more than one type of cell are cultured together in the same dish with either full or partial contact with each other.

6. Complex

The term complex as used herein refers to the association of a compound with an ion channel or enzyme for which the compound has a binding affinity.

7. Compound

For the purposes of the present disclosure the terms “compound,” “analog,” and “composition of matter” stand equally well for the chemical entities described herein, including all enantiomeric forms, diastereomeric forms, salts, and the like, and the terms “compound,” “analog,” and “composition of matter” are used interchangeably throughout the present specification.

8. Comprise

Throughout the description and claims of this specification, the word “comprise” and variations of the word, such as “comprising” and “comprises,” means “including but not limited to,” and is not intended to exclude, for example, other additives, components, integers or steps.

9. Components

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B—F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

10. Chemistry

The term “alkyl” refers to a linear or branched-chain saturated hydrocarbyl substituent (i.e., a substituent obtained from a hydrocarbon by removal of a hydrogen) containing from one to twenty carbon atoms; in one embodiment from one to twelve carbon atoms; in another embodiment, from one to ten carbon atoms; in another embodiment, from one to six carbon atoms; and in another embodiment, from one to four carbon atoms. Examples of such substituents include methyl, ethyl, propyl (including n-propyl and isopropyl), butyl (including n-butyl, isobutyl, sec-butyl and tert-butyl), pentyl, iso-amyl, hexyl and the like.

The term “alkenyl” refers to a linear or branched-chain hydrocarbyl substituent containing one or more double bonds and from two to twenty carbon atoms; in another embodiment, from two to twelve carbon atoms; in another embodiment, from two to six carbon atoms; and in another embodiment, from two to four carbon atoms. Examples of alkenyl include ethenyl (also known as vinyl), allyl, propenyl (including 1-propenyl and 2-propenyl) and butenyl (including 1-butenyl, 2-butenyl and 3-butenyl). The term “alkenyl” embraces substituents having “cis” and “trans” orientations, or alternatively, “E” and “Z” orientations.

The term “benzyl” refers to methyl radical substituted with phenyl, i.e., the following structure:

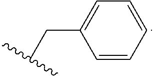 [SMILES: *Cc1ccccc1 ; "."]

The term “carbocyclic ring” refers to a saturated cyclic, partially saturated cyclic, or aromatic ring containing from 3 to 14 carbon ring atoms (“ring atoms” are the atoms bound together to form the ring). A carbocyclic ring typically contains from 3 to 10 carbon ring atoms. Examples include cyclopropyl, cyclobutyl, cyclopentyl, cyclopentenyl, cyclopentadienyl, cyclohexyl, cyclohexenyl, cyclohexadienyl, and phenyl. A “carbocyclic ring system” alternatively may be 2 or 3 rings fused together, such as naphthalenyl, tetrahydronaphthalenyl (also known as “tetralinyl”), indenyl, isoindenyl, indanyl, bicyclodecanyl, anthracenyl, phenanthrene, benzonaphthenyl (also known as “phenalenyl”), fluorenyl, and decalinyl.

The term “heterocyclic ring” refers to a saturated cyclic, partially saturated cyclic, or aromatic ring containing from 3 to 14 ring atoms (“ring atoms” are the atoms bound together to form the ring), in which at least one of the ring atoms is a heteroatom that is oxygen, nitrogen, or sulfur, with the remaining ring atoms being independently selected from the group consisting of carbon, oxygen, nitrogen, and sulfur.

The term “cycloalkyl” refers to a saturated carbocyclic substituent having three to fourteen carbon atoms. In one embodiment, a cycloalkyl substituent has three to ten carbon atoms. Examples of cycloalkyl include cyclopropyl, cyclobutyl, cyclopentyl and cyclohexyl.

The term “cycloalkyl” also includes substituents that are fused to a C₆-C₁₀ aromatic ring or to a 5-10-membered heteroaromatic ring, wherein a group having such a fused cycloalkyl group as a substituent is bound to a carbon atom of the cycloalkyl group. When such a fused cycloalkyl group is substituted with one or more substituents, the one or more substitutents, unless otherwise specified, are each bound to a carbon atom of the cycloalkyl group. The fused C₆-C₁₀ aromatic ring or to a 5-10-membered heteroaromatic ring may be optionally substituted with halogen, C₁-C₆ alkyl, C₃-C₁₀ cycloalkyl, or ═O.

The term “cycloalkenyl” refers to a partially unsaturated carbocyclic substituent having three to fourteen carbon atoms, typically three to ten carbon atoms. Examples of cycloalkenyl include cyclobutenyl, cyclopentenyl, and cyclohexenyl.

A cycloalkyl or cycloalkenyl may be a single ring, which typically contains from 3 to 6 ring atoms. Examples include cyclopropyl, cyclobutyl, cyclopentyl, cyclopentenyl, cyclopentadienyl, cyclohexyl, cyclohexenyl, cyclohexadienyl, and phenyl. Alternatively, 2 or 3 rings may be fused together, such as bicyclodecanyl and decalinyl.

The term “aryl” refers to an aromatic substituent containing one ring or two or three fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” also includes substituents such as phenyl, naphthyl and anthracenyl that are fused to a C₄-C₁₀ carbocyclic ring, such as a C₅ or a C₆ carbocyclic ring, or to a 4-10-membered heterocyclic ring, wherein a group having such a fused aryl group as a substituent is bound to an aromatic carbon of the aryl group. When such a fused aryl group is substituted with one more substituents, the one or more substitutents, unless otherwise specified, are each bound to an aromatic carbon of the fused aryl group. The fused C₄-C₁₀ carbocyclic or 4-10-membered heterocyclic ring may be optionally substituted with halogen, C₁-C₆ alkyl, C₃-C₁₀ cycloalkyl, or ═O. Examples of aryl groups include accordingly phenyl, naphthalenyl, tetrahydronaphthalenyl (also known as “tetralinyl”), indenyl, isoindenyl, indanyl, anthracenyl, phenanthrenyl, benzonaphthenyl (also known as “phenalenyl”), and fluorenyl.

In some instances, the number of carbon atoms in a hydrocarbyl substituent (e.g., alkyl, alkenyl, cycloalkyl, cycloalkenyl, aryl, etc.) is indicated by the prefix “Cₓ-C_{y}—,” wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₆-cycloalkyl refers to saturated cycloalkyl containing from 3 to 6 carbon ring atoms.

In some instances, the number of atoms in a cyclic substituent containing one or more heteroatoms (e.g., heteroaryl or heterocycloalkyl) is indicated by the prefix “X-Y-membered”, wherein x is the minimum and y is the maximum number of atoms forming the cyclic moiety of the substituent. Thus, for example, 5-8-membered heterocycloalkyl refers to a heterocycloalkyl containing from 5 to 8 atoms, including one ore more heteroatoms, in the cyclic moiety of the heterocycloalkyl.

The term “hydrogen” refers to hydrogen substituent, and may be depicted as —H.

The term “hydroxy” refers to —OH. When used in combination with another term(s), the prefix “hydroxy” indicates that the substituent to which the prefix is attached is substituted with one or more hydroxy substituents. Compounds bearing a carbon to which one or more hydroxy substituents include, for example, alcohols, enols and phenol.

The term “hydroxyalkyl” refers to an alkyl that is substituted with at least one hydroxy substituent. Examples of hydroxyalkyl include hydroxymethyl, hydroxyethyl, hydroxypropyl and hydroxybutyl.

The term “nitro” means —NO₂.

The term “cyano” (also referred to as “nitrile”) —CN, which also may be depicted:

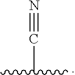 [SMILES: *C#N ; "."]

The term “carbonyl” means —C(O)—, which also may be depicted as:

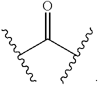 [SMILES: *C(*)=O]

The term “amino” refers to —NH₂.

The term “alkylamino” refers to an amino group, wherein at least one alkyl chain is bonded to the amino nitrogen in place of a hydrogen atom.

Examples of alkylamino substituents include monoalkylamino such as methylamino (exemplified by the formula —NH(CH₃)), which may also be depicted:

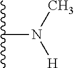 [SMILES: *NC]

and dialkylamino such as dimethylamino, (exemplified by the formula

—N(CH₃)₂, which may also be depicted:

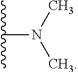 [SMILES: *N(C)C]

The term “aminocarbonyl” means —C(O)—NH₂, which also may be depicted as:

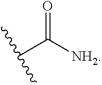 [SMILES: *C(N)=O]

The term “halogen” refers to fluorine (which may be depicted as —F), chlorine (which may be depicted as —Cl), bromine (which may be depicted as —Br), or iodine (which may be depicted as —I). In one embodiment, the halogen is chlorine. In another embodiment, the halogen is a fluorine.

The prefix “halo” indicates that the substituent to which the prefix is attached is substituted with one or more independently selected halogen substituents. For example, haloalkyl refers to an alkyl that is substituted with at least one halogen substituent. Where more than one hydrogen is replaced with halogens, the halogens may be the identical or different. Examples of haloalkyls include chloromethyl, dichloromethyl, difluorochloromethyl, dichlorofluoromethyl, trichloromethyl, 1-bromoethyl, fluoromethyl, difluoromethyl, trifluoromethyl, 2,2,2-trifluoroethyl, difluoroethyl, pentafluoroethyl, difluoropropyl, dichloropropyl, and heptafluoropropyl. Illustrating further, “haloalkoxy” refers to an alkoxy that is substituted with at least one halogen substituent. Examples of haloalkoxy substituents include chloromethoxy, 1-bromoethoxy, fluoromethoxy, difluoromethoxy, trifluoromethoxy (also known as “perfluoromethyloxy”), and 2,2,2-trifluoroethloxy. It should be recognized that if a substituent is substituted by more than one halogen substituent, those halogen substituents may be identical or different (unless otherwise stated).

The prefix “perhalo” indicates that each hydrogen substituent on the substituent to which the prefix is attached is replaced with an independently selected halogen substituent. If all the halogen substituents are identical, the prefix may identify the halogen substituent. Thus, for example, the term “perfluoro” means that every hydrogen substituent on the substituent to which the prefix is attached is replaced with a fluorine substituent. To illustrate, the term “perfluoroalkyl” refers to an alkyl substituent wherein a fluorine substituent is in the place of each hydrogen substituent. Examples of perfluoroalkyl substituents include trifluoromethyl (—CF₃), perfluorobutyl, perfluoroisopropyl, perfluorododecyl, and perfluorodecyl. To illustrate further, the term “perfluoroalkoxy” refers to an alkoxy substituent wherein each hydrogen substituent is replaced with a fluorine substituent. Examples ofperfluoroalkoxy substituents include trifluoromethoxy (—O—CF₃), perfluorobutoxy, perfluoroisopropoxy, perfluorododecoxy, and perfluorodecoxy.

The term “oxo” refers to ═O.

The term “oxy” refers to an ether substituent, and may be depicted as —O—.

The term “alkoxy” refers to an alkyl linked to an oxygen, which may also be represented as

—O—R, wherein the R represents the alkyl group. Examples of alkoxy include methoxy, ethoxy, propoxy and butoxy.

The term “alkylthio” means —S-alkyl. For example, “methylthio” is —S—CH₃. Other examples of alkylthio include ethylthio, propylthio, butylthio, and hexylthio.

The term “alkylcarbonyl” means —C(O)-alkyl. For example, “ethylcarbonyl” may be depicted as:

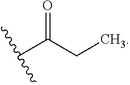 [SMILES: *C(=O)CC]

Examples of other alkylcarbonyl include methylcarbonyl, propylcarbonyl, butylcarbonyl, pentylcabonyl, and hexylcarbonyl.

The term “aminoalkylcarbonyl” means —C(O)-alkyl-NH₂. For

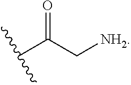 [SMILES: *C(=O)CN]

example, “aminomethylcarbonyl” may be depicted as:

The term “alkoxycarbonyl” means —C(O)—O-alkyl. For example, “ethoxycarbonyl” may be depicted as:

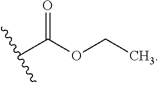 [SMILES: *C(=O)OCC]

Examples of other alkoxycarbonyl include methoxycarbonyl, ethoxycarbonyl, propoxycarbonyl, butoxycarbonyl, pentoxycarbonyl, and hexyloxycarbonyl. In another embodiment, where the carbon atom of the carbonyl is attached to a carbon atom of a second alkyl, the resulting functional group is an ester.

The terms “thio” and “thia” mean a divalent sulfur atom and such a substituent may be depicted as —S—. For example, a thioether is represented as “alkyl-thio-alkyl” or, alternatively, alkyl-S-alkyl.

The term “thiol” refers to a sulfhydryl substituent, and may be depicted as —SH.

The term “thione” refers to ═S.

The term “sulfonyl” refers to —S(O)₂—, which also may be depicted as:

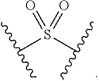 [SMILES: *S(*)(=O)=O]

Thus, for example, “alkyl-sulfonyl-alkyl” refers to alkyl-S(O)₂-alkyl. Examples of alkylsulfonyl include methylsulfonyl, ethylsulfonyl, and propylsulfonyl.

The term “aminosulfonyl” means —S(O)₂—NH₂, which also may be depicted as:

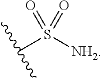 [SMILES: *S(N)(=O)=O]

The term “sulfinyl” or “sulfoxido” means —S(O)—, which also may be depicted as:

214.

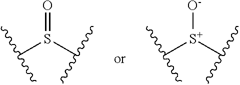

Thus, for example, “alkylsulfinylalkyl” or “alkylsulfoxidoalkyl” refers to alkyl-S(O)-alkyl. Exemplary alkylsulfinyl groups include methylsulfinyl, ethylsulfinyl, butylsulfinyl, and hexylsulfinyl.

The term “heterocycloalkyl” refers to a saturated or partially saturated ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur. A heterocycloalkyl alternatively may comprise 2 or 3 rings fused together, wherein at least one such ring contains a heteroatom as a ring atom (e.g., nitrogen, oxygen, or sulfur). In a group that has a heterocycloalkyl substituent, the ring atom of the heterocycloalkyl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heterocycloalkyl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom.

The term “heterocycloalkyl” also includes substituents that are fused to a C₆-C₁₀ aromatic ring or to a 5-10-membered heteroaromatic ring, wherein a group having such a fused heterocycloalkyl group as a substituent is bound to a heteroatom of the heterocyclocalkyl group or to a carbon atom of the heterocycloalkyl group. When such a fused heterocycloalkyl group is substituted with one more substituents, the one or more substitutents, unless otherwise specified, are each bound to a heteroatom of the heterocyclocalkyl group or to a carbon atom of the heterocycloalkyl group. The fused C₆-C₁₀ aromatic ring or to a 5-10-membered heteroaromatic ring may be optionally substituted with halogen, C₁-C₆ alkyl, C₃-C₁₀ cycloalkyl, or ═O.

The term “heteroaryl” refers to an aromatic ring structure containing from 5 to 14 ring atoms in which at least one of the ring atoms is a heteroatom (i.e., oxygen, nitrogen, or sulfur), with the remaining ring atoms being independently selected from the group consisting of carbon, oxygen, nitrogen, and sulfur. A heteroaryl may be a single ring or 2 or 3 fused rings. Examples of heteroaryl substituents include 6-membered ring substituents such as pyridyl, pyrazyl, pyrimidinyl, and pyridazinyl; 5-membered ring substituents such as triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. The term “heteroaryl” also includes pyridyl N-oxides and groups containing a pyridine N-oxide ring.

Examples of single-ring heteroaryls include furanyl, dihydrofiiranyl, tetradydrofilranyl, thiophenyl (also known as “thiofiuranyl”), dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiaediazolyl, oxathiazolyl, oxadiazolyl (including oxadiazolyl, 1,2,4-oxadiazolyl (also known as “azoximyl”), 1,2,5-oxadiazolyl (also known as “furazanyl”), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl (including 1,2-pyranyl or 1,4-pyranyl), dihydropyranyl, pyridinyl (also known as “azinyl”), piperidinyl, diazinyl (including pyridazinyl (also known as “1,2-diazinyl”), pyrimidinyl (also known as “1,3-diazinyl” or “pyrimidyl”), or pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)), oxazinyl (including 1,2,3-oxazinyl, 1,3,2-oxazinyl, 1,3,6-oxazinyl (also known as “pentoxazolyl”), 1,2,6-oxazinyl, or 1,4-oxazinyl), isoxazinyl (including o-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl or 1,3,5,2-oxadiazinyl), morpholinyl, azepinyl, oxepinyl, thiepinyl, and diazepinyl.

Examples of 2-fused-ring heteroaryls include, indolizinyl, pyrindinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl, indolyl, isoindolyl, indoleninyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

Examples of 3-fused-ring heteroaryls or heterocycloalkyls include 5,6-dihydro-4H-imidazo[4,5,1-ij]quinolin e, 4,5-dihydroimidazo[4,5,1-hi]indole, 4,5,6,7-tetrahydroimidazo[4,5,1-jk][l]benzazepine, and dibenzofuranyl.

Other examples of fused-ring heteroaryls include benzo-fused heteroaryls such as indolyl, isoindolyl (also known as “isobenzazolyl” or “pseudoisoindolyl”), indoleninyl (also known as “pseudoindolyl”), isoindazolyl (also known as “benzpyrazolyl”), benzazinyl (including quinolinyl (also known as “l-benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3-benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl,” “thionaphthenyl,” or “benzothiofuranyl”), isobenzothienyl (also known as “isobenzothiophenyl,” “isothionaphthenyl,” or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, or 3,1,4-benzoxazinyl), benzisoxazinyl (including 1,2-benzisoxazinyl or 1,4-benzisoxazinyl), tetrahydroisoquinolinyl, carbazolyl, xanthenyl, and acridinyl.

The term “heteroaryl” also includes substituents such as pyridyl and quinolinyl that are fused to a C₄-C₁₀ carbocyclic ring, such as a C₅ or a C₆ carbocyclic ring, or to a 4-10-membered heterocyclic ring, wherein a group having such a fused aryl group as a substituent is bound to an aromatic carbon of the heteroaryl group or to a heteroatom of the heteroaryl group. When such a fused heteroaryl group is substituted with one more substituents, the one or more substitutents, unless otherwise specified, are each bound to an aromatic carbon of the heteroaryl group or to a heteroatom of the heteroaryl group. The fused C₄-C₁₀ carbocyclic or 4-10-membered heterocyclic ring may be optionally substituted with halogen, C₁-C₆ alkyl, C₃-C₁₀ cycloalkyl, or ═O.

The term “ethylene” refers to the group —CH₂—CH₂—. The term “ethynelene” refers to the group —CH═CH—. The term “propylene” refers to the group —CH₂—CH₂—CH₂— The term “butylene” refers to the group —CH₂—CH₂—CH₂—CH₂—. The term “methylenoxy” refers to the group —CH₂—O—. The term “methylenethioxy” refers to the group —CH₂—S—. The term “methylenamino” refers to the group —CH₂—N(H)—. The term “ethylenoxy” refers to the group —CH₂—CH₂—O—. The term “ethylenethioxy” refers to the group —CH₂—CH₂—S—. The term “ethylenamino” refers to the group —CH₂—CH₂—N(H)—.

A substituent is “substitutable” if it comprises at least one carbon, sulfur, oxygen or nitrogen atom that is bonded to one or more hydrogen atoms. Thus, for example, hydrogen, halogen, and cyano do not fall within this definition. If a substituent is described as being “substituted,” a non-hydrogen substituent is in the place of a hydrogen substituent on a carbon, oxygen, sulfur or nitrogen of the substituent. Thus, for example, a substituted alkyl substituent is an alkyl substituent wherein at least one non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each non-hydrogen substituent may be identical or different (unless otherwise stated).

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent. One exemplary substituent may be depicted as —NR′R″ wherein R′ and R″ together with the nitrogen atom to which they are attached, may form a heterocyclic ring. The heterocyclic ring formed from R′ and R″ together with the nitrogen atom to which they are attached may be partially or fully saturated. In one embodiment, the heterocyclic ring consists of 3 to 7 atoms. In another embodiment, the heterocyclic ring is selected from the group consisting of pyrrolyl, imidazolyl, pyrazolyl, triazolyl, tetrazolyl, isoxazolyl, pyridyl and thiazolyl.

This specification uses the terms “substituent,” “radical,” and “group” interchangeably. If a group of substituents are collectively described as being optionally substituted by one or more of a list of substituents, the group may include: (1) unsubstitutable substituents, (2) substitutable substituents that are not substituted by the optional substituents, and/or (3) substitutable substituents that are substituted by one or more of the optional substituents. If a substituent is described as being optionally substituted with up to a particular number of non-hydrogen substituents, that substituent may be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 non-hydrogen substituents, then any heteroaryl with less than 3 substitutable positions would be optionally substituted by up to only as many non-hydrogen substituents as the heteroaryl has substitutable positions. To illustrate, tetrazolyl (which has only one substitutable position) would be optionally substituted with up to one non-hydrogen substituent. To illustrate further, if an amino nitrogen is described as being optionally substituted with up to 2 non-hydrogen substituents, then the nitrogen will be optionally substituted with up to 2 non-hydrogen substituents if the amino nitrogen is a primary nitrogen, whereas the amino nitrogen will be optionally substituted with up to only 1 non-hydrogen substituent if the amino nitrogen is a secondary nitrogen.

A prefix attached to a multi-moiety substituent only applies to the first moiety. To illustrate, the term “alkylcycloalkyl” contains two moieties: alkyl and cycloalkyl. Thus, a C₁-C₆-prefix on C₁-C₆-alkylcycloalkyl means that the alkyl moiety of the alkylcycloalkyl contains from 1 to 6 carbon atoms; the C₁-C₆-prefix does not describe the cycloalkyl moiety. To illustrate further, the prefix “halo” on haloalkoxyalkyl indicates that only the alkoxy moiety of the alkoxyalkyl substituent is substituted with one or more halogen substituents. If the halogen substitution may only occur on the alkyl moiety, the substituent would be described as “alkoxyhaloalkyl.” If the halogen substitution may occur on both the alkyl moiety and the alkoxy moeity, the substituent would be described as “haloalkoxyhaloalkyl.”

When a substituent is comprised of multiple moieties, unless otherwise indicated, it is the intention for the final moiety to serve as the point of attachment to the remainder of the molecule. For example, in a substituent A-B-C, moiety C is attached to the remainder of the molecule. In a substituent A-B-C-D, moiety D is attached to the remainder of the molecule. Similarly, in a substituent aminocarbonylmethyl, the methyl moiety is attached to the remainder of the molecule, where the substituent may also be depicted as

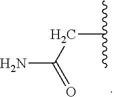 [SMILES: *CC(N)=O]

In a substituent trifluoromethylaminocarbonyl, the carbonyl moiety is attached to the remainder of the molecule, where the substituent may also be depicted as

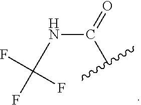 [SMILES: *C(=O)NC(F)(F)F]

If substituents are described as being “independently selected” from a group, each substituent is selected independent of the other. Each substituent therefore may be identical to or different from the other substituent(s).

11. Comprise

Throughout the description and claims of this specification, the word “comprise” and variations of the word, such as “comprising” and “comprises,” means “including but not limited to,” and is not intended to exclude, for example, other additives, components, integers or steps.

12. Control

The terms “control” or “control levels” or “control cells” are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a predetermined standard.

13. Higher

The terms “higher,” “increases,” “elevates,” or “elevation” or like terms or variants of these terms, refer to increases above basal levels, e.g., as compared a control. The terms “low,” “lower,” “reduces,” “decreases” or “reduction” or variation of these terms, refer to decreases below basal levels, e.g., as compared to a control. For example, basal levels are normal in vivo levels prior to, or in the absence of, or addition of an agent such as an agonist or antagonist to activity. For example, decreases or increases can be used to describe the binding of a molecule to a receptor. In this context, decreases would describe a situation of where the binding could be defined as having a Kd of 10⁻⁹ M, if this interaction decreased, meaning the binding lessened, the Kd could decrease to 10⁻⁶ M. It is understood that wherever one of these words is used it is also disclosed that it could be 1%, 5%, 10%, 20%, 50%, 100%, 500%, or 1000% increased or decreased from a control.

14. Inhibit

By “inhibit” or other forms of inhibit means to hinder or restrain a particular characteristic. It is understood that this is typically in relation to some standard or expected value, in other words it is relative, but that it is not always necessary for the standard or relative value to be referred to. For example, “inhibits phosphorylation” means hindering or restraining the amount of phosphorylation that takes place relative to a standard or a control.

15. Maintaining

The word “maintaining” or like words refers to continuing a state. In the context of a treatment, maintaining can be refer to less than 20%, 15%, 10%, 9%, 8%, 7%, 6%, 5%, 4%, 3%, 2%, 1%, or 0.1% change from a control, such a basal level, often a level in the absence of a treatment or in the presence of treatment with a placebo or standard.

16. Material

Material is the tangible part of something (chemical, biochemical, biological, or mixed) that goes into the makeup of a physical object.

17. Modulate

The term modulate or like terms refers to its standard meaning of increasing or decreasing.

18. Substance

A substance or like terms is any physical object. A material is a substance. Molecules, ligands, markers, cells, proteins, and DNA can be considered substances. A machine or an article would be considered to be made of substances, rather than considered a substance themselves.

19. Molecule

As used herein, the terms “molecule” or like terms refers to a biological or biochemical or chemical entity that exists in the form of a chemical molecule or molecule with a definite molecular weight. A molecule or like terms is a chemical, biochemical or biological molecule, regardless of its size.

Many molecules are of the type referred to as organic molecules (molecules containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, small molecule, receptors, antibodies, and lipids. When appropriate, one or more of these more descriptive terms (many of which, such as “protein,” themselves describe overlapping groups of molecules) will be used herein because of application of the method to a subgroup of molecules, without detracting from the intent to have such molecules be representative of both the general class “molecules” and the named subclass, such as proteins. Unless specifically indicated, the word “molecule” would include the specific molecule and salts thereof, such as pharmaceutically acceptable salts.

20. Optionally

“Optional” or “optionally” means that the subsequently described event or circlmstance may or may not occur, and that the description includes instances where said event or circumstance occurs and instances where it does not.

21. Prevent

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibits phosphorylation is disclosed, then reduces and prevents phosphorylation are also disclosed.

22. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that this data, represents endpoints and starting points, and ranges for any combination of the data points. For example, if a particular datum point “10” and a particular datum point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

23. Reduce

By “reduce” or other forms of reduce means lowering of an event or characteristic. It is understood that this is typically in relation to some standard or expected value, in other words it is relative, but that it is not always necessary for the standard or relative value to be referred to. For example, “reduces phosphorylation” means lowering the amount of phosphorylation that takes place relative to a standard or a control.

24. References

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

25. Specifically interacts

Specifically interacts or like terms means that the interaction is beyond a background interaction. The background interaction can be determined by for example looking at the interaction with serum albumin.

26. Subject

As used throughout, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. The subject can also be a non-human.

27. Tissue

Tissue or like terms refers to a collection of cells. Typically a tissue is obtained from a subject.

28. Treating

“Treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease. In certain situations a treatment can inadvertantly cause harm.

29. Therapeutically effective

The term “therapeutically effective” means that the amount of the composition used is of sufficient quantity to treat a subject as defined herein.

30. Synergy

The term “synergistic effect” or “synergy” as used herein means that the therapeutic effect of a combination comprising two or more agents is more effective than the therapeutic effect of a treatment where only a single agent alone is applied. Further, a synergistic effect of a combination of two or more agents permits the use of lower dosages of one or more of the agents and/or less frequent administration of said agents to a patient. The ability to utilize lower dosages of an agent and/or to administer said agent less frequently reduces the toxicity associated with the administration of said agent to a patient without reducing the efficacy of said agent in the prevention, management or treatment of the diseases or conditions. In addition, a synergistic effect can result in improved efficacy of agents in the prevention, management or treatment of the diseases or conditions. Moreover, a synergistic effect of a combination of two or more agents may avoid or reduce adverse or unwanted side effects associated with the use of either agent alone.

31. Pharmacophore

The term “pharmacophore”, as used herein, refers to a structural element in a drug or bioactive molecule that is critical for biological interaction to its biological target and its subsequent biological effects.

32. Treating or preventing

“Treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease. In certain situations a treatment can inadvertantly cause harm. The term “preventing” refers to the ability of a compound or composition of the invention to prevent a disease identified herein in patients diagnosed as having the disease or who are at risk of developing such disease. In this context, preventing includes the delaying the onset of the disease relative to a control. This term also encompasses preventing further progression of the disease in patients who are already suffering from or have symptoms of such disease. As used herein the term “treating” means both treatment having a curing or alleviating purpose and treatment having a preventive purpose. The treatment can be made either acutely or chronically. It is understood that treatment can mean a reduction or one or more symptoms or characteristics by at least 10%, 20%, 30%, 40%, 50%, 60%, 70%, 80%, 90%, 95%, 99%, 99.9%, 99.99%, 100%, relative to a control.

### G. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Examples of Biological Testing

Disclosed compounds were tested in varies biological assays. Specifically, biological testing results of the following compounds (structures with correspondingly designated identification numbers) are illustrated below. The results are intended to illustrate the present invention without posing any limitation to it.

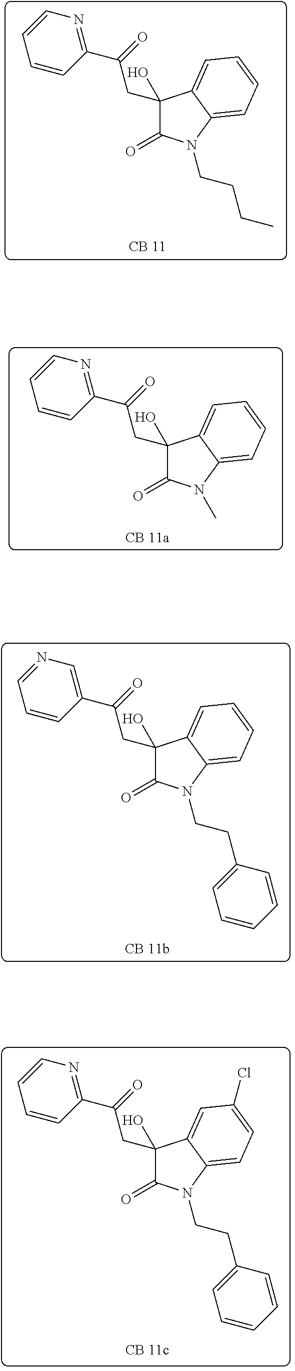

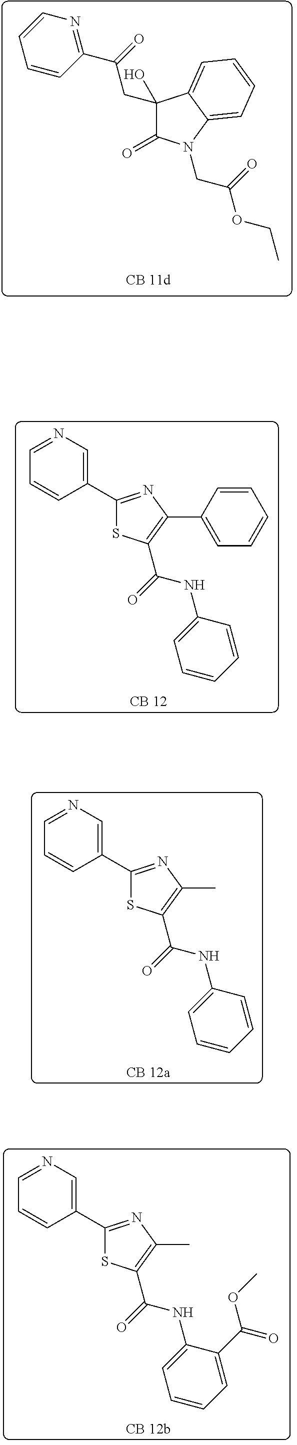

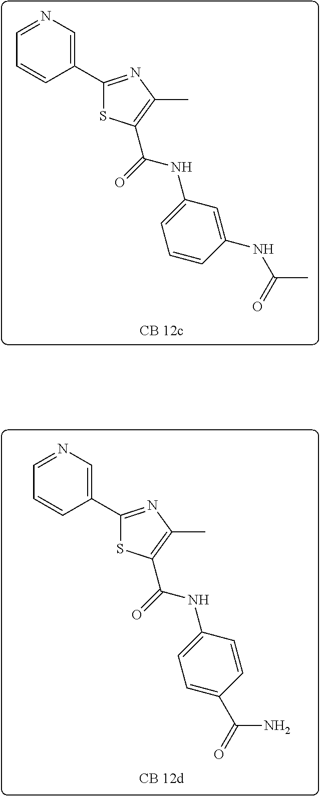 [SMILES: CC(=O)Nc1cccc(NC(=O)c2sc(-c3cccnc3)nc2C)c1 ; "CB 12c" ; Cc1nc(-c2cccnc2)sc1C(=O)Nc1ccc(C(N)=O)cc1 ; "CB 12d"]

a) In Vitro Assays

(1) Calcium-Induced Mitochondrial Damage Assay

(a) Materials and Methods

Materials and methods for the results listed below were as described herein for this date or related data.

(b) Results

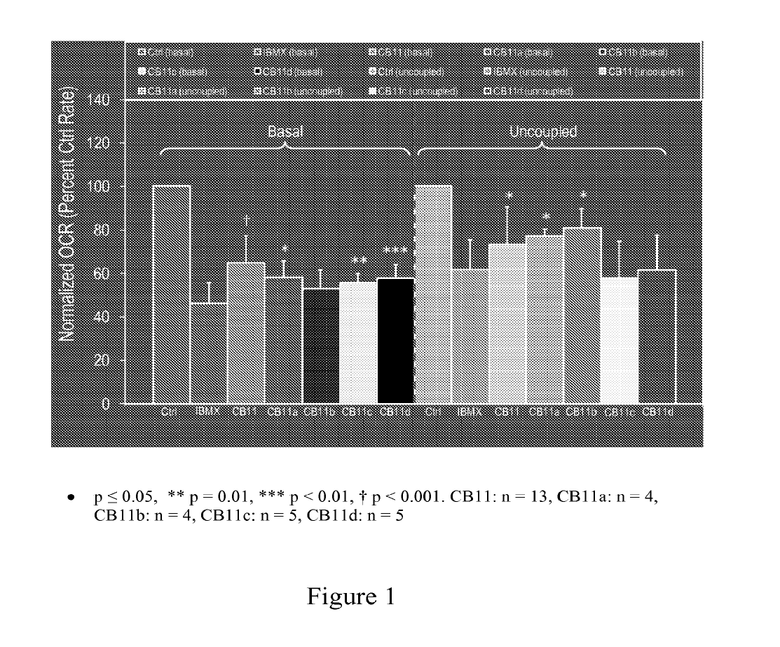

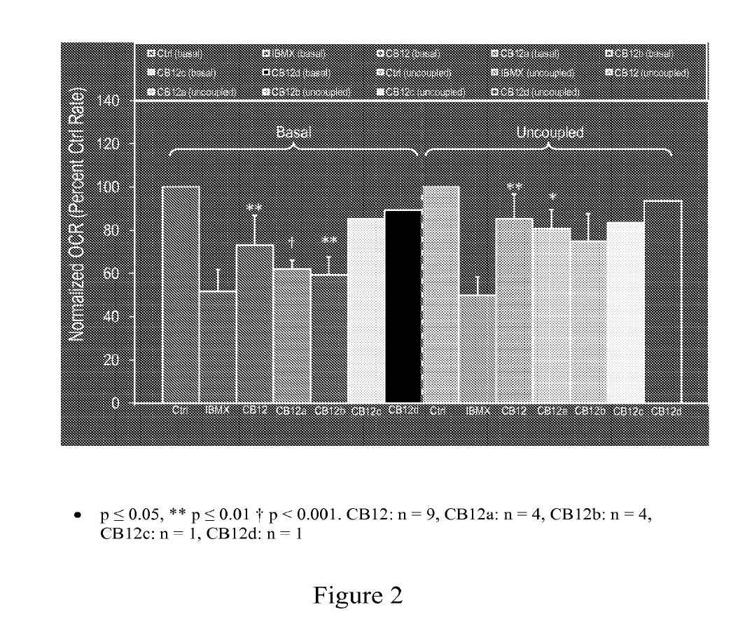

As shown in FIG. 1 and FIG. 2 below, compounds CB11, CB11a, CB11b, CB1c, CB11d and IBMX (see FIG. 1); compounds CB12, CB12a, CB12b, CB12c, CB12d and IBMX (see FIG. 2) were tested against the calcium-induced mitochondrial damage assay. IBMX is a membrane-permeable inhibitor of 3-phosphodiesterase which results in a buildup of cGMP. An excess of cGMP increases the number of cGMP-gated cation channels in an open configuration, allowing an influx of Ca2+ into the cell, leading to mitochondrial damage and eventual cell death. A concentration of 500-600 μM IBMX was found to cause about a 50% decrease in both basal and maximal (FCCP-uncoupled) respiratory capacity in 661W cells (OCR=oxygen consumption rate, a measure of mitochondrial metabolic function), indicative of calcium-induced mitochondrial damage. Pretreatment with either 1 μM CB11 (as well as CB11a, CB11b, CB11c, and CB11d) for 1 h prior to the addition of IBMX led to improvement of both basal and maximal OCR. Also, pretreatment with 1 μM CB12 (as well as CB12a, CB12b, CB12c, and CB12d) for 1 h prior to the addition of IBMX led to improvement of both basal and maximal OCR. Therefore, these compounds were shown to be effective in preventing the loss of mitochondrial respiratory capacity, which protects and increases the mitochondrial metabolic function, and ultimately prevents the mitochondrial damage and dysfunction.

(2) Rd1 Mouse Retinal Organ Culture Assay

(a) Materials and Methods

Materials and methods for the results listed below were as described herein for this date or related data.

(b) Results

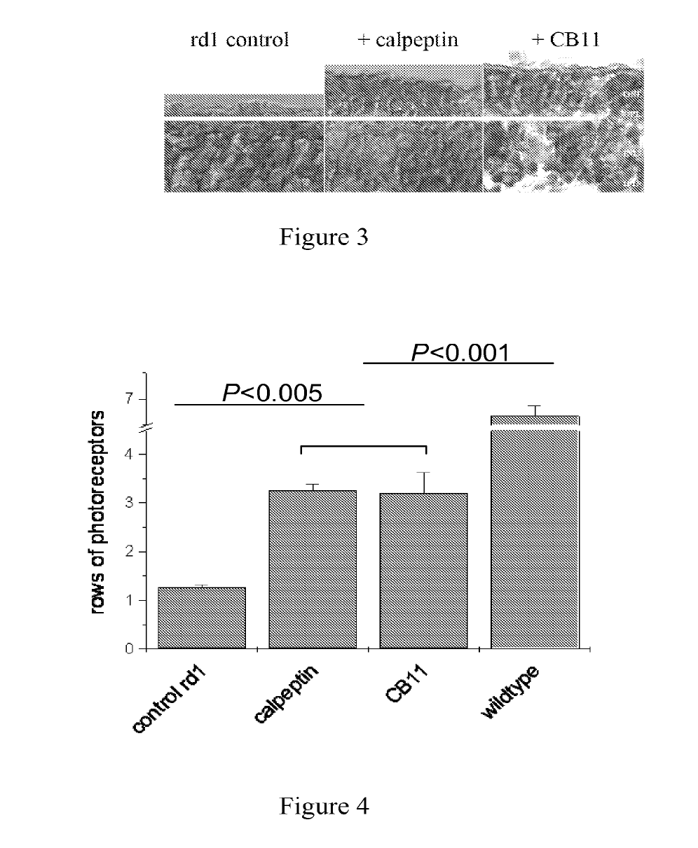

As shown in FIG. 3 and FIG. 4 below, compound CB11 was tested against rd1 Mouse retinal organ culture assay. FIG. 3 shows frozen sections of rd1 retina-RPE sandwich cultures grown in culture from post-natal day (P)10 through P21. Compounds were replaced with media changes (every 48 hrs). In FIG. 3, the left-hand image is a vehicle-treated control retina from an rd1 mouse; the middle image is of an rd1 retina treated with calpeptin, which blocks calpain, preventing apoptotic cell death (positive control). The right-hand image of an rd1 retina treated with CB11, which was found to protect rd1 photoreceptors comparable to calpeptin. FIG. 4 quantifies and summarizes the images from FIG. 3, and it shows that an untreated rd1 retina has about 1 row of photoreceptors remaining at P21; calpeptin-treated retinas have about 3.5 rows, CB11-treated retinas also have about 3.5 rows, and wild-type control retinas have almost 7 rows. Therefore, CB11 was shown to be effective in protecting retinal photoreceptors against calcium-induced degeneration (protecting cell death).

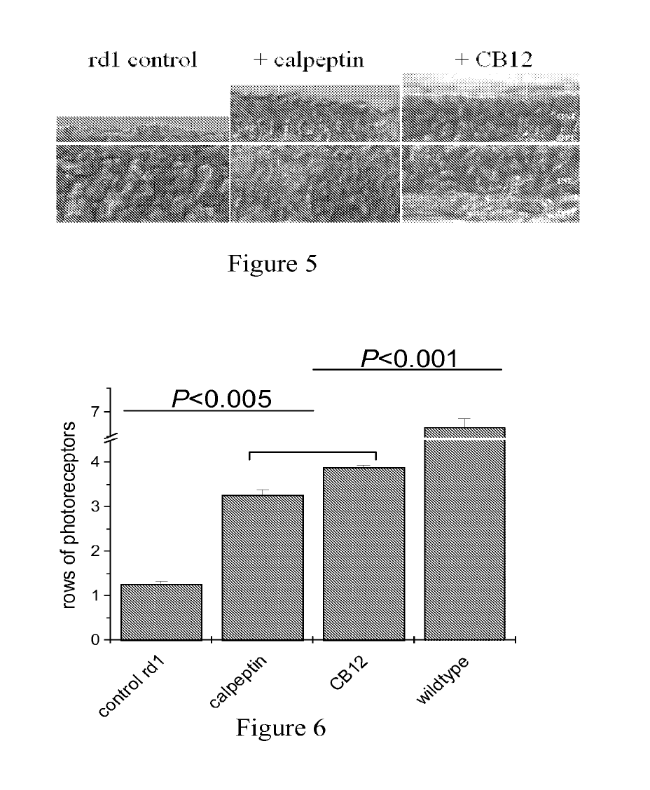

As shown in FIG. 5 and FIG. 6 below, compound CB12 was tested against rd1 Mouse retinal organ culture assay. FIG. 5 shows frozen sections of rd1 retina-RPE sandwich cultures grown in culture from post-natal day (P)10 through P21. Compounds were replaced with media changes (every 48 hrs). In FIG. 3, the left-hand image is a vehicle-treated control retina from an rd1 mouse; the middle image is of an rd1 retina treated with calpeptin, which blocks calpain, preventing apoptotic cell death (positive control). The right-hand image of an rd1 retina treated with CB121, which was found to protect rd1 photoreceptors comparable to calpeptin. FIG. 6 quantifies and sunmmarizes the images from FIG. 5, and it shows that an untreated rd1 retina has about 1 row of photoreceptors remaining at P21; calpeptin-treated retinas have about 3.5 rows, CB12-treated retinas also have about 4 rows, and wild-type control retinas have almost 7 rows. Therefore, CB12 was shown to be effective in protecting retinal photoreceptors against calcium-induced degeneration (protecting cell death).

b) In Vivo Assays

(1) Materials and Methods

Materials and methods for the results listed below were as described herein for this date or related data.

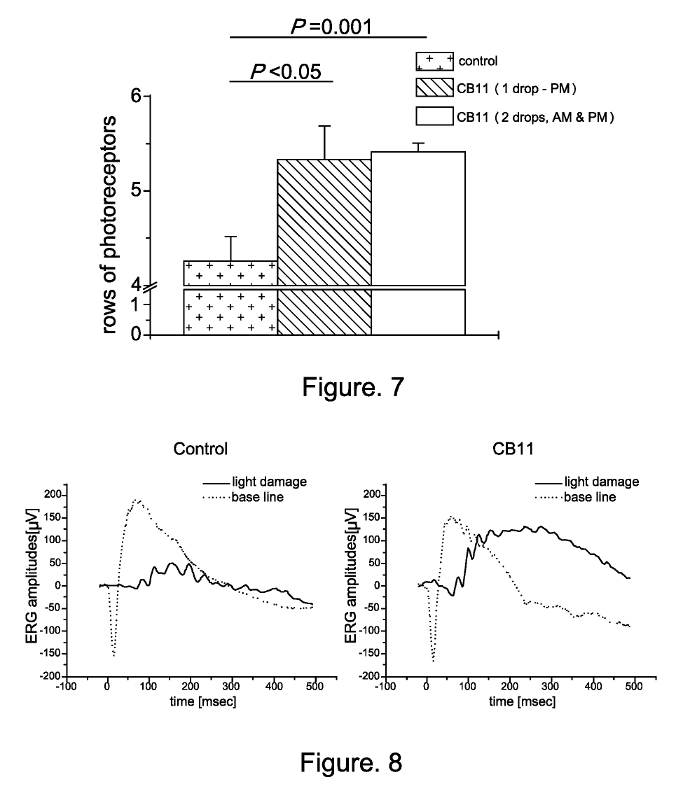

CB11 was formulated into an aqueous solution containing 1 mM CB11 dissolved in 2% ethanol, 0.5% Brij-78, and 0.9% NaCl in water. The animal model used was the constant light model in Balb/c mice, in which the rod photoreceptors die of oxidative stress, resulting in ˜50% cell loss within 10 days. Animals were treated with CB11 by administering 10 μL eyedrops at the time-points indicated in FIG. 7 (1 drop in the PM, or one eyedrop every 12 hours) over the 10 days of continuous light. After 10 days, the mice were sacrificed and the rows of photoreceptors were manually counted as indicated in FIG. 7.

(2) Results

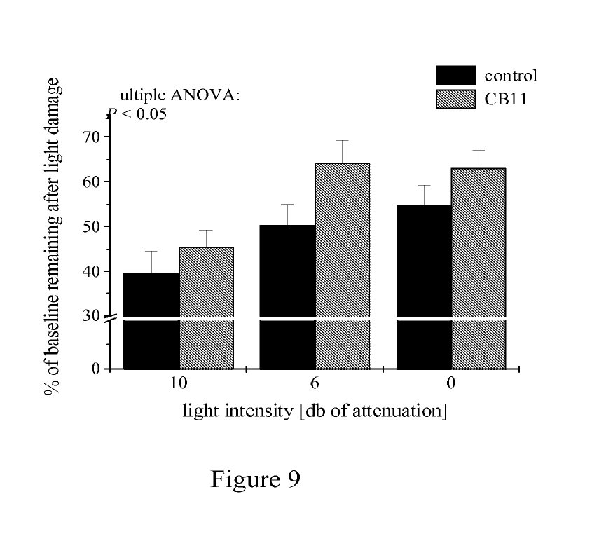

FIGS. 7-9 show that when CB11 was administered as daily eyedrops (10 μL of 1 mM stock) during 10 days light damage (continuous exposure) in mice, CB11 was found to be effective in protecting against oxidative stress-induced photoreceptor degeneration at both a structural level (see FIG. 7) and functional level (see FIGS. 8 and 9). Specifically, FIG. 7 shows that animal treated by CB11 had more rows of photoreceptors than untreated animal after 10 days light damage. FIG. 8 shows the result of Electroretinography (ERG). ERG is a tool to measure the response of the entire retina to a flash of light, using corneal surface electrodes. The negative deflection is the response of the photoreceptors, while the positive deflection is a response of the first set of interneurons, the rod bipolar cells (and hence tests synaptic transmission). Each animal was tested prior to light damage (baseline, red trace) and after light damage (black trace). Two examples are shown of mice after 10 days of eyedrops containing either saline (control) or CB11 formulation (CB11). Control animals had significantly smaller ERG amplitudes compared to those receiving daily CB11 treatment, demonstrating that mice receiving CB11 had significantly better retinal function (i.e., could see better) after 10 days of continuous light damage than vehicle treated mice. FIG. 9 quantifies the percent of the baseline ERG amplitude (from FIG. 8) by retinas of sacrificed mice in response to different intensities of light. FIG. 9 shows that the ERG response at each light intensity was significantly improved by CB11.

### 2. Examples of Computational Modeling in Generating the Pharmacophores

a) Mechanism and Methodology

Materials and methods for the results listed below were as described herein for this date or related data.

b) Results

The following compounds (structures with correspondingly designated identification numbers shown below) are used by the computational modeling to generate the pharmacophores of the disclosed compounds.

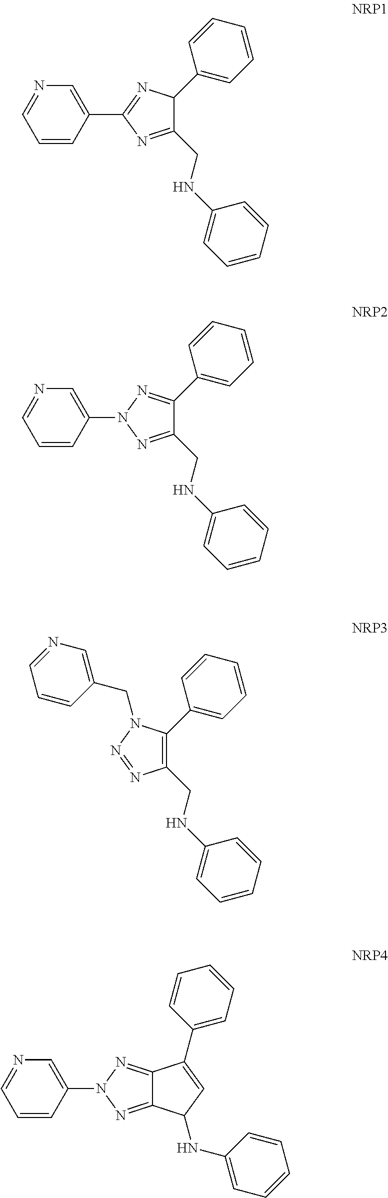 [SMILES: c1ccc(NCC2=NC(c3cccnc3)=NC2c2ccccc2)cc1 ; c1ccc(NCc2nn(-c3cccnc3)nc2-c2ccccc2)cc1 ; c1ccc(NCc2nnn(Cc3cccnc3)c2-c2ccccc2)cc1 ; C1=C(c2ccccc2)c2nn(-c3cccnc3)nc2C1Nc1ccccc1]

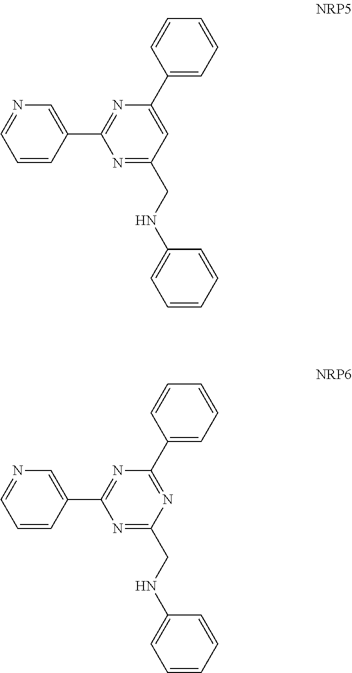 [SMILES: c1ccc(NCc2cc(-c3ccccc3)nc(-c3cccnc3)n2)cc1 ; c1ccc(NCc2nc(-c3ccccc3)nc(-c3cccnc3)n2)cc1]

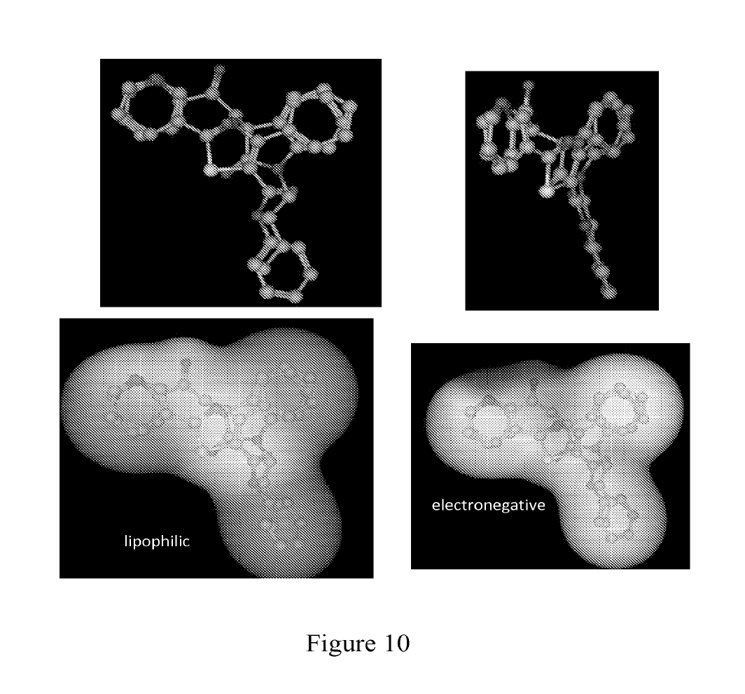

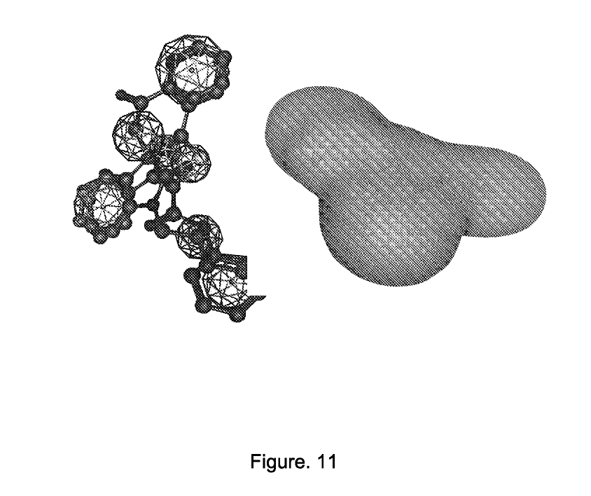

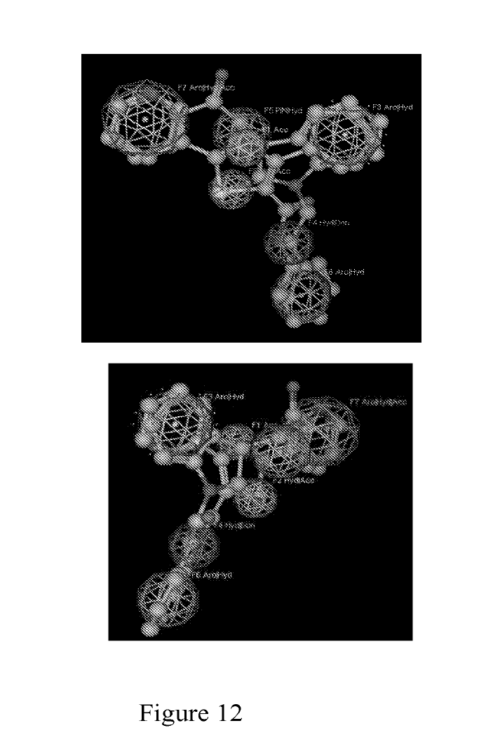

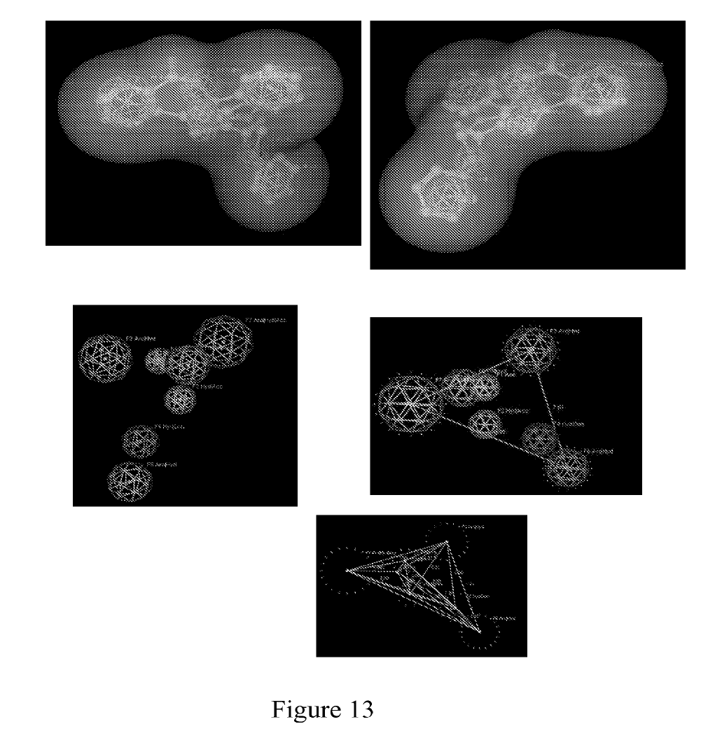

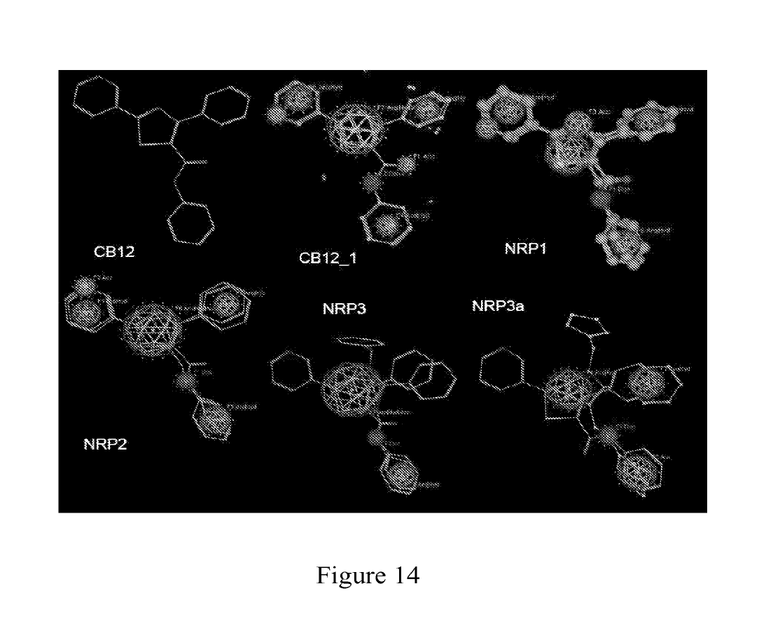

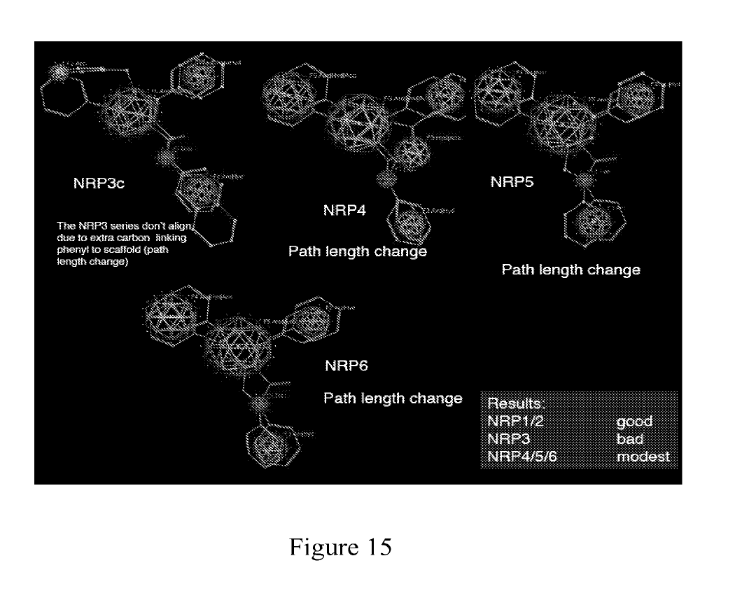

FIG. 10 shows the overlap of liphophilic and electronegative properties between CB11 and CB12. FIG. 11 shows spacial overlap of physicochemical features such as hydrophobicity, hydrogen bond donor/acceptors, polar regions between CB11 and CB12. FIG. 12 shows the seven point consensus pharmacophores between CB11 and CB12. The two molecules define a single pharmacophore with 100% overlap of seven key features, shown are two orientations of the pharmacophores. Note that the central cores are dissimilar reflecting the differences between the indole and thiazole scaffolds included in CB11 and CB12 respectively. The seven point consensus pharmacophores (referred to as F1 to F7 in FIGS. 11-13) are: (1) one or more hydrogen bond acceptor and/or donor; (2) one or more hydrogen bond acceptor and/or donor; (3) one or more hydrogen bond donor and/or acceptor groups; and/or one or more hydrophobic groups; (4) one or more hydrogen bond donor and/or acceptor groups; (5) one or more hydrophobic groups; (6) one or more hydrophobic groups; and (7) one or more hydrophobic groups. FIG. 13 shows the spatial connection and arrangement of the seven point consensus pharmacophores discussed above. The pharmacophores are independent of scaffold and the corresponding “binding site” of the target species.

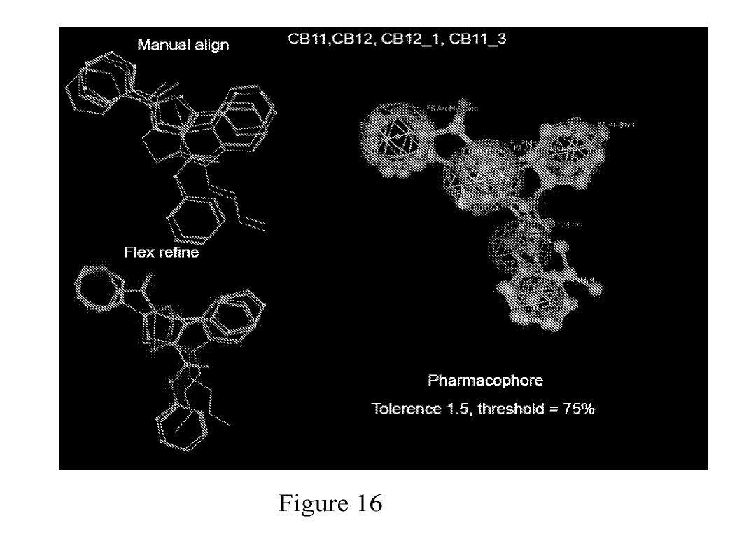

FIG. 16 shows that CB11, CB12, CB12_1 and CB11_3 are overlapped under the seven point consensus pharmacophores discussed above.

### 3. Example 4

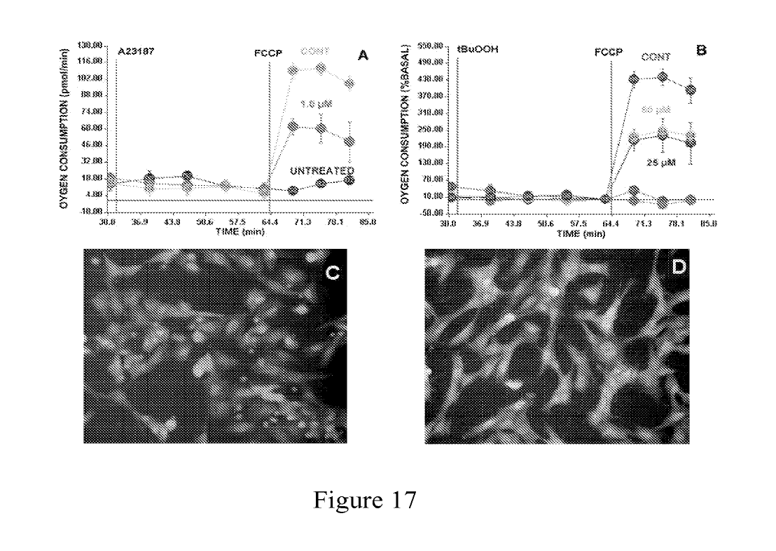

In preliminary experiments the energy metabolism of 661W cells using the XF24. 661W cells were analyzed and produce large amounts of lactic acid from glucose oxidation [Winkler B S, Starnes C A, Sauer M W, Firouzgan Z, Chen S C: Cultured retinal neuronal cells and Muller cells both show net production of lactate. Neurochem Int 2004, 45(2-3):311-320]. It was also found that 661W cells have very high oxygen consumption rates and, perhaps most intriguing; these cells readily metabolize lactate but not exogenous pyruvate, which is consistent with an operative pyruvate shuttle. Thus, the 661W cells exhibit many metabolic phenotypes seen in intact photoreceptors. To examine whether the metabolic responses were predictive of cell death due to calcium or oxidant stress, the cells were exposed to calcium ionophore A23187 or the oxidant tert-butylhydroperoxide (tBuOOH) on the XF24 instrument for 30 min after which some of the treated cells were exposed to the protonophore FCCP to uncouple the mitochondrial membrane potential. The uncoupling attenuates ATP production and causes the mitochondria to dramatically increase oxygen consumption in an attempt to recover the lost ATP production capacity. The increase in oxygen consumption is a measure of the total mitochondrial capacity or reserve. Both A23187 and tBuOOH caused significant loss of mitochondrial capacity 30 min after treatment as measured from the FCCP response (FIG. 17).

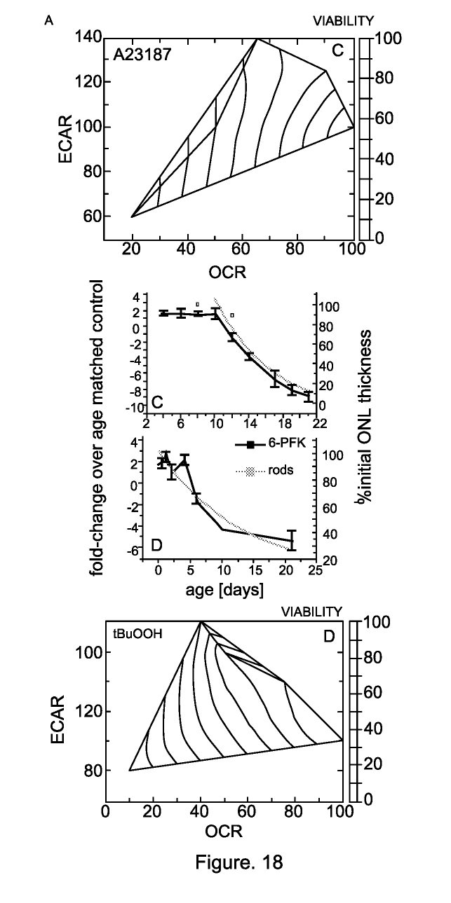

The cells were then returned to the incubator and analyzed for cell viability via dye exclusion 24 h after treatment (FIG. 17C,D). It was found that the higher doses of both calcium ionophore and hydroperoxide caused significant death at 24 h (up to 80%); the cell viability was >95% immediately following the experiment. In addition, the higher concentrations produced profound changes in metabolic phenotype as evidenced in the cell's responses to the uncoupler FFCP. When these data were analyzed as shown in FIG. 18A,B, it was found that both the calcium and oxidant stresses produced very similar correlations between the two measures of metabolic capacity, changes in OCR (oxygen consumption, an indicator of mitochondrial respiration) and ECAR (extracellular acid release, the result of glycolysis) due to uncoupling measured at 30 min and cell viability at 24 h. In both cases, reduced OCR and ECAR values are predictive of cell death although at intermediate concentrations, where cell death is still occurring, the ECAR rates actually increase, particularly for tBuOOH treatments.

The XF24 assay platform provides a robust measure of metabolic dysfunction that is predictive of long-term cell death. A similar observation was made when analyzing mRNA levels for 6-phospho-fructokinase (6-PFK; rate limiting enzyme for glycolysis) during photoreceptor degeneration in mouse models in which photoreceptor degeneration is triggered by calcium (rd1) or oxidative stress (light damage). Prior to cell death, 6-PFK levels are elevated presumably to generate ATP to protect the cells against the ionic imbalance, but during cell death, 6-PFK levels are suppressed [Lohr H R, Kuntchithapautham K, Sharma A K, Rohrer B: Multiple, parallel cellular suicide mechanisms participate in photoreceptor cell death. Exp Eye Res 2006, 83(2):380-389](FIG. 18C,D; see also Appendix 3). The increase and subsequent decrease of PFK expression tracks with the changes in ECAR measured in 661W cells.

### 4. Example 5

### XF24 Assay Validation

In preliminary studies, the direct oxidant tert-butylhydroperoxide (tBuOOH) was used to induce oxidant stress and the agent A23187, a calcium ionophore that binds to and carries Ca²⁺ across cellular membranes, including mitochondrial membranes (Abbott, B. J., Fukuda, D. S., Dorman, D. E., Occolowitz, J. L., Debono, M., and Farhner, L. (1979) Antimicrob. Agents Chemother. 16(6), 808-812). Because A23187 rapidly increases intracellular and intra-mitochondrial calcium concentrations, it triggers Ca²⁺-mediated programmed cell death (2. Orrenius, S., Zhivotovsky, B., and Nicotera, P. (2003) Nat. Rev. Mol. Cell. Biol. 4, 552-565; Hajnóczky, G., Davies, E., and Madesh, M. (2003) Biochem. Biophys. Res. Commun. 304, 445-454). One could argue that these two agents are akin to hammers rather than the more subtle, chronic oxidant and calcium stressors seen in degenerative processes. The XF24 assay was validated using two more physiologically relevant stressors. To induce calcium stress, 3-Isobutyl-1-methyl xanthine (IBMX), a non-selective phosphodiesterase (PDE) inhibitor was used (4. Zhang, X., Feng, Q., and Cote, R. H. (2005) Invest Ophthalmol. Vis. Sci. 46(9), 3060-3066). Inhibition of PDEs causes increases in the intracellular cAMP and cGMP concentrations leading to slow activation cGMP-gated cation channels and an increase of Ca²⁺ flux into the cell (5. Yarfitz, S., and Hurley, J. B. (1994) J. Biol. Chem. 269(20), 14329-14332; Koutalos, Y., and Yau, K.-W. (1996) Trends Neurosci. 19, 73-81). IBMX has been shown to invoke a transient elevation of Ca²⁺ concentration by releasing Ca²⁺ from intracellular stores in neurons (Usachev, Y., and Verkhratsky, A. (1995) Cell Calcium 17(3), 197-206), and exposure of photoreceptor cells to 5 mM IBMX caused decreased response amplitude and desensitization of the cells similar to the effects of long-term Ca²⁺ treatment (8. Lipton, S. A., Rasmussen, H., and Dowling, J. E. (1977) J. Gen. Physiol. 70, 771-791). Paraquat (Pq²⁺) is a divalent bipyridinium cation, known primarily for its use as an herbicide. Pq²⁺ crosses cell and mitochondrial membranes based on membrane potential, and at the mitochondrial level in mammals, Pq²⁺ is reduced by complex I of the mitochondrial membrane (9. Cochemé, H. M., and Murphy, M. P. (2008) J. Biol. Chem. 283(4), 1786-1798). Upon reduction, the paraquat cation radical (Pq^{+•}) is formed, which rapidly reacts with oxygen to form superoxide (O₂^{-•}) (10. Hassan, H. M. (1984) Methods Enzymol. 105, 523-532), a primary source of intracellular oxidative stress, and regenerates Pq²⁺. Pq²⁺ is an intracellular redox cycler that simulates in vivo conditions of oxidative stress (11. Fukushima, T., Tanaka, K., Lim, H., and Moriyama, M. (2002) Environ. Health Prevent. Med. 7, 89-94; 12.

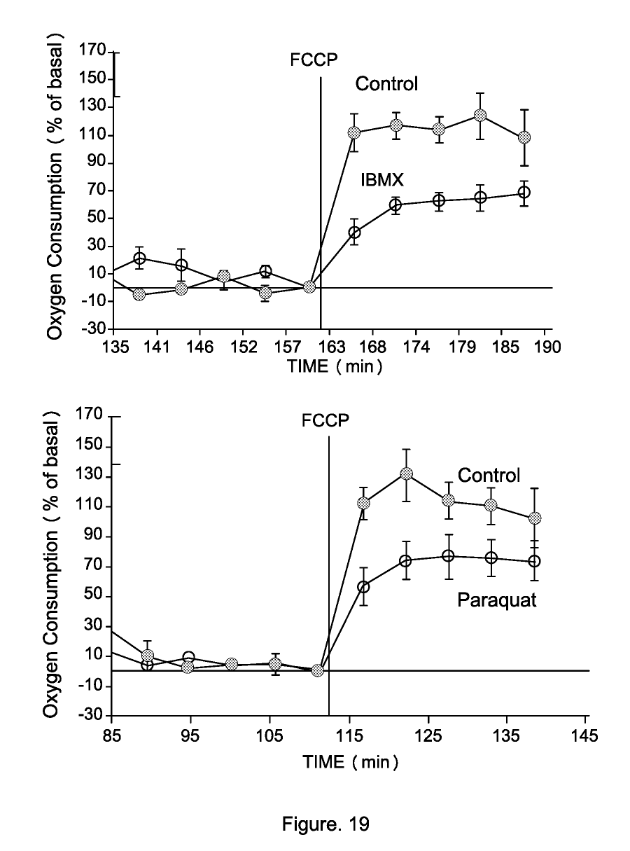

Medrano C J, Fox D A. (1995) Exp. Eye Res. 61(3):273-84). It was found that both IBMX and paraquat produce the same types of loss in mitochondrial capacity as had originally been observed with the more potent, direct stress agents tBuOOH and A23187. FIG. 19 provides representative data for the mitochondrial oxygen consumption of 661W cells as they are exposed to IBX and paraquat. The agents caused little or no direct effect on basal respiration rates (not shown), but, when the mitochondria are uncoupled (FCCP treatment), the untreated cells show and approximately 100% increase in respiration (i.e., their capacity is about twice of their basal). In contrast, the cells that had been treated with 1 μM IBMX for 20 min show about a 50% loss in metabolic capacity as measured from the FCCP response.

### 5. Example 6

### High Information Content Imaging

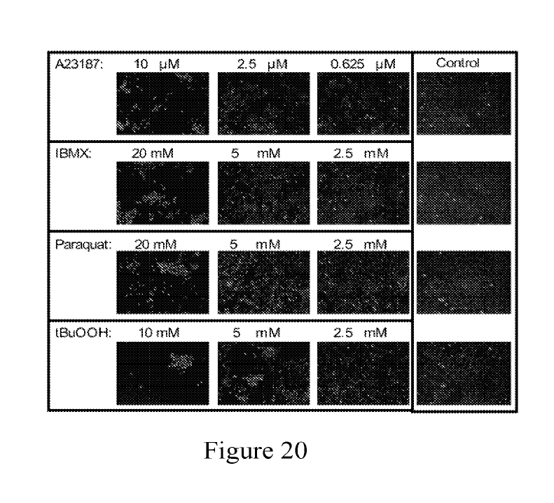

A significant challenge in metabolism studies is the selection of techniques that can provide relevant information regarding the cellular health, mitochondrial content, and other parameters that could have adverse effects on interpretation of data. The XF24 instrument provides an unforeseen window into cellular metabolism but other views into the cell are needed and the use of the GE Health Sciences INCell 1000 analyzer to complement the disclosed metabolic assays is disclosed. The use of high-resolution, automated cell imaging in metabolic studies has not been reported and disclosed herein the Seahorse Biosciences XF24 instrument with imaging have been hybridized together. It was found that the imaging greatly facilitates the assessment of viability. Using the two nuclear-permeable dyes, discrimination between live versus dead cells is possible. Hoechst 33342 (blue) stains all cells, whereas propidium iodide (red) stains only dead cells, and also enables the distinction between apoptotic versus nectrotic cell types (FIG. 20). The viability assays measured with the INCell for 661W cells exposed to different concentrations of A23187, IBMX, paraquat, and tBuOOH show a strong correlation with the XF24 measurements, as had been reported for tBuOOH and A23187 herein. These results confirm the generality of the original observations and they provide further support of the role of mitochondrial degeneration and eye diseases.

### 6. Example 7

### Library Screening

ChemBridge, Inc. has a large (>700,000) commercially available chemical library and a DIVERSet library was obtained, which is a subset of compounds selected for diversity and favorable drug-like properties. Because the DIVERSet library is much too large for lower throughput, high-content screens, single end-point assays amenable to high-throughput were developed and disclosed herein. The 50,000 compounds were initially screened with random pools of 10 compounds at 1 μM each (total concentration 10 μM) followed by deconvolution of pools that exhibited activity. A viability assay using treatments with A23187 to search for molecules in the library that protect from acute calcium toxicity was developed and disclosed herein. The concentration of A23187 was selected to give about 50% death and pools that protected against death were identified. The assay format was optimized and validated using calpeptin, a caspase inhibitor as a positive control. In brief, 661W cells were maintained in DMEM supplemented with 10% FBS. For the assay, 100 μL of 70,000 cells/mL cells were seeded into each well of 96 well plates using DMEM supplemented with 5% FBS. Cells were then allowed to grow to confluency for 48 hours. Library compounds were added in 2 μL containing 10 compounds at 20 M each. Ionophore A23187 was then added in 1 μL for a final concentration of 1 μM and after 24 hours cells were analyzed for viability using the MTS assay according to the manufactures protocol (Promega CellTiter 96® Cat.#G5421). Screening compounds were also tested in the absence of ionophore to determine cellular toxicity. As a positive control, Calpain Inhibitor I (Sigma) at 50 μM was able to reverse ionophore effects. Each drug combo was performed in duplicate and positive hits were repeated and then deconvoluted to individual compounds at multiple doses to identify actives. The assay identified 12 molecules that provided protection against A23187 at 1 μM or lower.

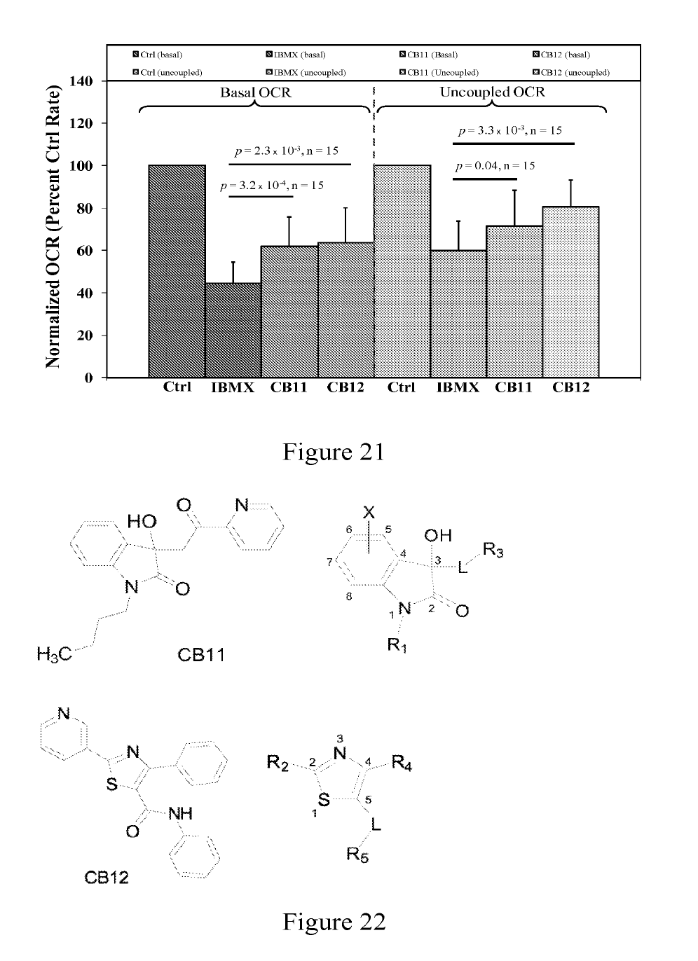

High-throughput screening of large chemical libraries is often fraught with a large number of hits that prove to be non-specific or inactive when taking into more specific, secondary screens. A higher stringency for hit selection than is commonly used was performed to avoid the less productive process of filtering through a large number of poor leads. It is notable that the complete ChemBridge library contains over 700,000 molecules that are searchable based on chemical structure, physicochemical properties, etc. To identify the active species present in the initial pool of 12 compounds were screened and then chemical similarity searches on the larger 700,000 member library was performed to identify other molecules that define pharmacophores. This strategy has more efficiently lead to agents that block retinal degeneration. Also, this strategy enabled quick identification of a small number of agents that that were moved into more sophisticated cellular and physiological models so as to better validate the mechanisms. For example, in the high-throughput screen a very toxic, non-specific calcium ionophore assay was used which gives a rather broad readout of cell “viability”. In the secondary assays the specificity and stringency were increased via use of more specific calcium stressor, 3-isobutyl-1-methylxanthine (IBMX) and a more relevant metabolic read-out, loss of respiratory capacity as measured from the attenuation of the uncoupled respiratory rate as measured with the Seahorse instrument. This assay format was used to evaluate the 12 initial lead compounds and are pleased to report that several of these agents do indeed protect against loss in respiratory capacity due to IBMX exposure. For example, representative data for a secondary screen are shown in FIG. 21. Here it was found that two agents, CB11 and CB12 (FIG. 22) provided significant protection against calcium-induced loss in respiratory capacity.

### 7. Example 8

### Identified Molecules

The molecules CB3, CB11, and CB12 were evaluated using computational tools to develop a pharmacophore. The primary tool for these analyses was the Molecular Operating Environment (MOE) software package produced by Chemical Computing Group, Inc. The MOE package is an integrated platform containing applications in bioinformatics, cheminformatics, QSAR, pharmacophore modeling, structure-based design and HTS discovery support. The three molecules were automatically aligned in 3D using the pharmacophore elucidation features in MOE. In brief, multiple conformers of each molecule were generated using a stochastic, parallelized fragment-based approach and these were aligned based on maximizing overlap of similar physicochemical features and minimizing collective volume. However, the first generation alignments of multiple structures were quickly improved with manual refinement that often includes flipping a ring or rotamer, excluding an enantiomer, or excluding a molecule that confounds multiple alignments, often because of path length differences. In this case, CB3 was dropped from an alignment of CB11 and CB12.

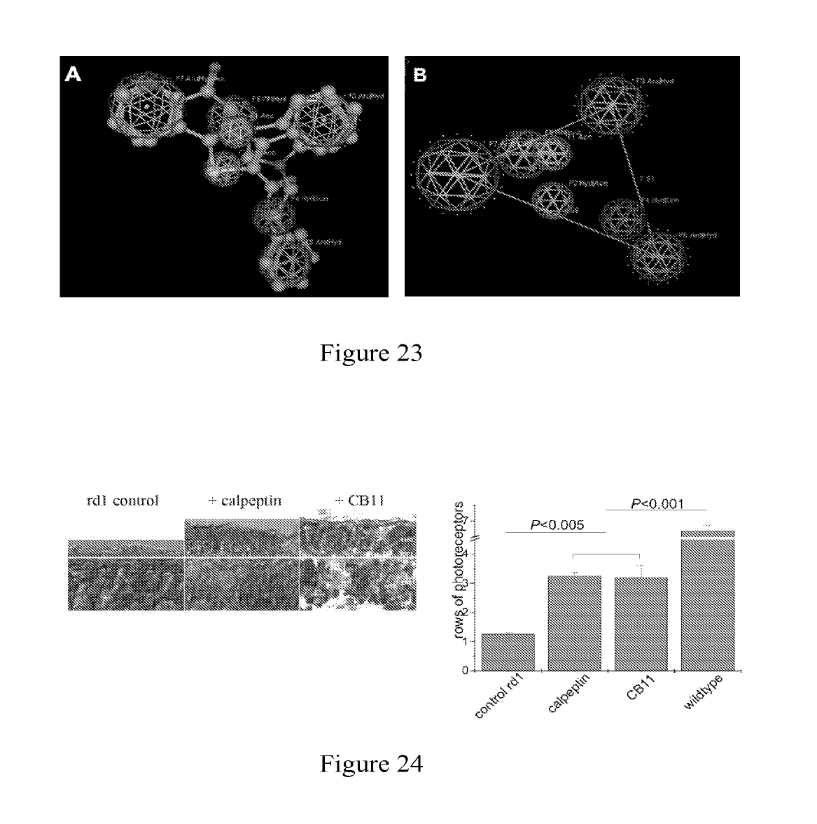

Once a pharmacophore is initially defined, it can be used to identify new molecules that can be tested to refine the pharmacophore and begin QSAR. Ultimately, the pharmacophore enables identification of additional molecules that fit the class claimed to have cytoprotective capacity via protection of mitochondrial capacity. It was found that CB11 and CB12 overlap in chemical space to define a single pharmacophore with nearly 100% overlap of seven physicochemical features (FIG. 23). After refining the pharmacophore, it was found that CB3 also overlaps the pharmacophore space.

### 8. Example 9

### Retinal Development and Degeneration of the rd1 Mouse Retina is Recapitulated in Organ Culture

A retina-RPE explant culture was established to analyze rod development under controlled conditions, avoiding drug delivery issues and systemic involvement, starting the culture by P₁₁. At P₁₁, all retinal cells have been born and have migrated into their final position within the retina [1. Rohrer, B., et al., Role of neurotrophin receptor TrkB in the maturation of rod photoreceptors and establishment of synaptic transmission to the inner retina. J. Neurosci., 1999. 19(20): p. 8919-8930], but their final maturations is incomplete. These early postnatal retinas grown with the RPE attached continue to grow and mature in culture, resulting in an anatomical configuration within these explants that is comparable to that of retinal tissue In vivo.

Rod degeneration of the rd1 mouse retina also occurs ex vivo and recapitulate the time course seen in vivo, resulting in the loss of almost all photoreceptors by P21 [2. Ogilvie, J. M., et al., A reliable method for organ culture of neonatal mouse retina with long-term survival. J. Neurosci. Methods, 1999. 87(1): p. 57-65]. Thus, the disclosed ex vivo RPE/retinal explants mimic in vivo under-defined experimental culture conditions.

### 9. Example 10

### Rod Degeneration in the Rd1 Mouse Retina can be Ameliorated by CB11 and CB12

A normal mouse retina grown in culture at P21 contains on average 6.7+0.2 vertical rows of rods, whereas the rd1 mouse retina only contains 1.26±0.2 rows. Rd1 mouse organ cultures were exposed to 1 mM CB11 or CB12 and compared from P₁₁ to P21, replenishing the compounds with each media replacement, using calpeptin as a positive control since calpain activation has been shown to be one of the main mediators of cell death in this model [3.

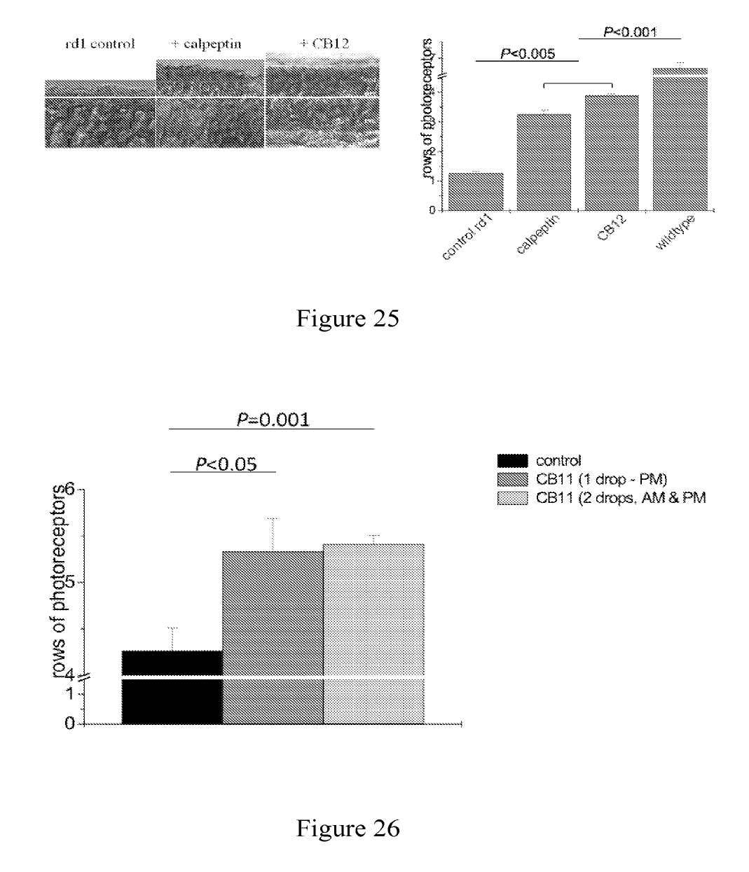

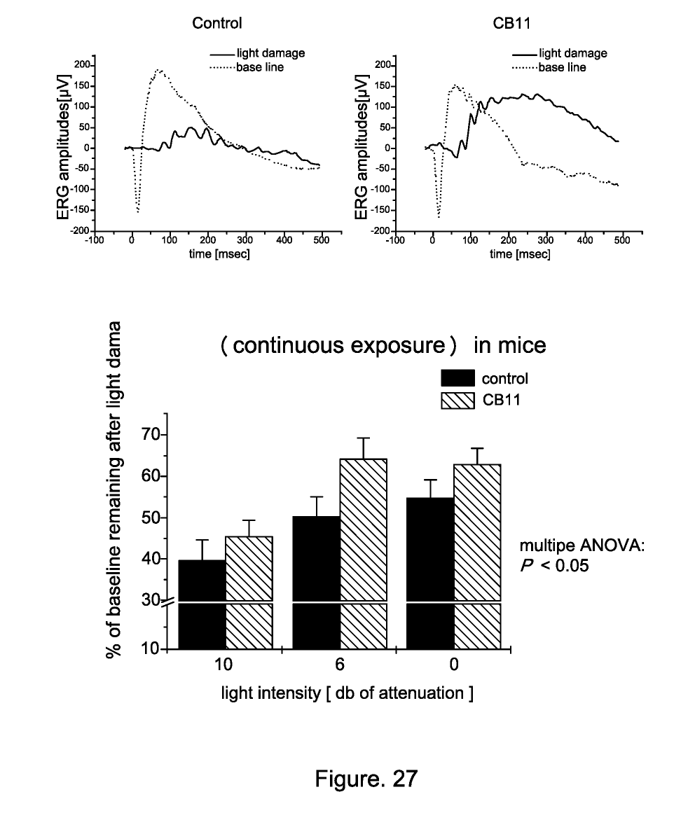

Sharma, A. K. and B. Rohrer, Calcium-induced calpain mediates apoptosis via caspase-3 in a mouse photoreceptor cell line. J Biol Chem, 2004. 279(34): p. 35564-72]. Calpeptin-treated retinas contained on average 3.3±0.3 rows of photoreceptors (P<0.0005); which was comparable to the results obtained by CB11 (3.2±0.6 and CB12: 3.8±0.01, see FIG. 24 and FIG. 25).

### 10. Example 11

### Light Damage as a Model of Oxidative Stress

Light as an environmental factor has been shown to be toxic to rod photoreceptors if the retina is exposed to high light levels over a long period of time (reviewed in [4. Penn, J. S. and D. H. Anderson, Effects of light history on the rat retina. Progress in Retinal Research, ed. C. G. Osborne N N. 1992, NY: Pergamon Press. 75-98]); and oxidative stress has been implicated as the main trigger for cell death. In particular, oxidative damage has been detected by immimohistochemistry, detecting the presence of oxidized and tyrosine-phosphorylated proteins [5. Tanito, M., et al., Attenuation of retinal photooxidative damage in thioredoxin transgenic mice. Neurosci Lett, 2002. 326(2): p. 142-6] as well as the upregulation of endogenous antioxidants such as thioredoxin and glutathione peroxidase [5. Tanito, M., et al., Attenuation of retinal photooxidative damage in thioredoxin transgenic mice. Neurosci Lett, 2002. 326(2): p. 142-6; 6. Ohira, A., et al., Glutathione peroxidase induced in rat retinas to counteract photic injury. Invest Ophthalmol Vis Sci, 2003. 44(3): p. 1230-6]. Likewise, exogenous antioxidants have been found to protect the rodent retina from light damage [7. Li, Z. Y., et al., Amelioration of photic injury in rat retina by ascorbic acid: a histopathologic study. Invest Ophthalmol Vis Sci, 1985. 26(11): p. 1589-98; 8. Noell, W. K., et al., Ascorbate and dietary protective mechanisms in retinal light damage of rats: electrophysiological, histological and DNA measurements. Prog Clin Biol Res, 1987. 247: p. 469-83]. Addition indirect evidence for the involvement of oxidative stress in photoreceptor degeneration has been provided by treatment of photodamaged retinas with antioxidants such as dimethylthiourea [9. Specht, S., et al., Damage to rat retinal DNA induced in vivo by visible light. Photochem Photobiol, 1999. 69(1): p. 91-8], or the treatment of N-methyl-N-nitrosourea (MNU)-challenged rats with the antioxidant DHA [10. Moriguchi, K., et al., Suppression of N-methyl-N-nitrosourea-induced photoreceptor apoptosis in rats by docosahexaenoic acid. Ophthalmic Res, 2004. 36(2): p. 98-105].

Photoreceptors from albino animals are very sensitive to constant light, lacking the RPE pigment to protect them. Fluorescent light at an illuminance of approximately 115-175 ft-c is sufficient to reduce the numbers of photoreceptors by 50% within 10 days and to 1 row within 2-3 weeks in young adult (3-month old) albino mice [11. Faktorovich, E. G., et al., Basic fibroblast growth factor and local injury protect photoreceptors from light damage in the rat. J. Neurosci., 1992. 12(9): p. 3554-3567; 12. Rohrer, B., et al., Lack of p75 receptor does not protect photoreceptors from light-induced cell death. Exp Eye Res, 2003. 76(1): p. 125-9].

To test the potential therapeutic efficacy, eyedrops were formulated (see Material and Methods), applied them twice daily throughout the period of light exposure, and assessed their effect on the light-induced degeneration of photoreceptor cells morphologically and electrophysiologically, 10 days after the onset of the CL exposure.

In control BALB/c mice, constant light resulted in the elimination of ˜50% of the photoreceptors (average retina score: 4.3±0.25 rows of photoreceptors), whereas the mice treated with CB11 eyedrops retained significantly more photoreceptors cells (5.4±0.36 rows of photoreceptors; P<0.001, FIG. 26).

Likewise, ERG analysis confirmed that while after light-damage, the ERG consisted of only a measurable b-wave (50.2±4.8 mV) and no a-wave in the BALB/c animals, the ERG of the treated mice exhibited a significantly more preserved b-wave (64±5.1 mV) (P<0.05).

### 11. Example 12

### Methods Used to Collect CB11 and CB12 Data

a) Constant Light Exposure.

Photoreceptors from albino animals are very sensitive to constant light, lacking the RPE pigment to protect them. Fluorescent light at an illuminance of approximately 115-175 ft-c is sufficient to reduce the numbers of photoreceptors to 1 row within 2-3 weeks in young adult (3-month old) albino mice [1. Faktorovich, E. G., et al., Basic fibroblast growth factor and local injury protect photoreceptors from light damage in the rat. J. Neurosci., 1992. 12(9): p. 3554-3567; 2. Rohrer, B., et al., Lack of p75 receptor does not protect photoreceptors from light-induced cell death. Exp Eye Res, 2003. 76(1): p. 125-9].

b) Electroretinography

Mice were anesthetized using xylazine and ketamine. Pupils were dilated with a drop of phenylephrine HCl (2.5%) and tropicamide (1%). Body temperature was stabilized via a DC-powered heating pad, and held at 37° C. A needle ground electrode was placed in the tail and a reference needle electrode in the forehead. ERG responses were measured using a contact lens containing a gold-ring electrode [3. Bayer, A. U., et al., Evaluation of different recording parameters to establish a standard for flash electroretinography in rodents. Vis. Res., 2001. 41(17): p. 2173-2185] held in place by a drop of methyl-cellulose. ERGs were recorded with the UTAS-2000 (LKC Technologies, Inc., Gaithersburg, Md.) system, using a Grass strobe-flash stimulus [4. Gresh, J., et al., Structure-function analysis of rods and cones in juvenile, adult, and aged C57bl/6 and Balb/c mice. Vis Neurosci, 2003. 20(2): p. 211-20]. Stimulus light intensity were controlled using neutral density filters. The responses were recorded at a gain of 2 k using a notch filter at 60 Hz, and are band-pass filtered between 0.1 and 1500 Hz. Stimulus paradigms. The unattenuated strength of the flash in this photostimulator (as calibrated by the manufacturer; in units of time-integrated luminance) is 2.48 photopic cd-s/m² at the dome's inner-surface. Animals were dark-adapted overnight and ERGs were recorded. Rods will be analyzed in response to single-flash stimuli of increasing light intensity, chosen to be within the linear range of the amplification coefficient (i.e., the gain of the biochemical activation stages of the rod signal transduction cascade) in the mouse signal transduction cascade [5. Tyubarsky, A. T. and E. N. Pugh Jr., Recovery phase of the murine rod photoresponse reconstructed from electroretinographic recordings. J. Neurosci., 1996. 16(2): p. 563-571]. The single-flash responses were an average of at least 3 flashes with an inter-stimulus interval (ISI) of 15 seconds to 2 minute (lowest intensity to highest, respectively). The different ISIs ensure that ERG amplitudes at a given intensity were identical between the first and the last flash. Data analysis. For all ERG recordings, a-wave amplitude were measured from baseline to a-wave trough; b-wave amplitude was measured from a-wave trough or baseline to peak of b-wave, and implicit time were measured from onset of stimulus to a-wave trough or b-wave peak.

c) Retinotypic cultures

All chemicals used for organ cultures were tissue culture grade and were purchased from Invitrogen (Carlsbad, Calif.). Retina-RPE (retina pigment-epithelium) cultures were grown by means of the interface technique according to published protocols [6. Rohrer, B. and J. M. Ogilvie, Retarded outer segment development in TrkB knockout mouse retina organ culture. Mol Vis, 2003. 9: p. 18-23; Ogilvie, J. M., et al., A reliable method for organ culture of neonatal mouse retina with long-term survival. J. Neurosci. Methods, 1999. 87(1): p. 57-65; Pinzon-Duarte, G., et al., Cell differentiation, synaptogenesis, and influence of the retinal pigment epithelium in a rat neonatal organotypic retina culture. Vision Res, 2000. 40(25): p. 3455-65] with modifications. All preparations were performed under a laminar flow hood. Pups were deeply anesthetized by hypothermia and decapitated. Heads were rinsed in 70% ethanol and eyeballs collected and placed in ice-cold Hanks balanced salt solution plus glucose (6.5 g/L). To collect the retina with RPE, eyes were incubated in 1 mL of media containing cystein (0.035 mg) and papain (20 Units) at 37° C. for 15 minutes. Enzymatic activity was stopped by adding media plus 10% fetal calf serum. The anterior chamber was removed, followed by the lens and vitreous. Using a pair of #5 forceps, the retina with the RPE attached was then carefully dissected free from the choroid and sclera. Relaxing cuts were made into the retina-RPE sandwich to flatten the tissues. The tissues were then transferred to the upper compartment of a Costar Transwell chamber using a drop of Neurobasal medium (Invitrogen), RPE-layer face-down. The drop of fluid was used to flatten-out the retina, by gently spreading the drop of liquid with the fused-end of a glass Pasteur pipette. Neurobasal media supplemented with 1% Ni and 2% B-27 supplements were placed in the lower compartment. The cultures were kept in an incubator (5% CO₂, balanced air, 100% humidity, at 37° C.). The medium was changed every two days at which time agents were replenished. No antimicotics or antibiotics were required.

d) Histology

Semi-thin plastic and frozen or paraffin sections were used.

Semi-Thin Plastic Sections.

Animals were deeply anaesthetized with CO₂ and perfused transcardially with Karnofsky fixative (2% paraformaldehyde, 4% glutaraldehyde in phosphate buffered saline (PBS), pH 7.4) [4. Gresh, J., et al., Structure-function analysis of rods and cones in juvenile, adult, and aged C57bl/6 and Balb/c mice. Vis Neurosci, 2003. 20(2): p. 211-20]. Eyes were hemisected through three landmarks (superior and inferior oblique, optic nerve) to guarantee the same orientation in all eyes. After tissue osmication (2% in PB, for 1 hour), dehydration (quick rinses in 50%, 75%, 95%, 100% and 1 hour in absolute ethanol) and propylene oxide treatment (10 min) eyecups were embedded in Epon/Araldite and cured at 80° C. for 6-8 hours. Semithin (1 μm) sections were cut on a microtome, stained with toluidine blue solution (1% tol blue, 1% borax in dH₂O) and coverslipped using DPX mounting medium. In this preparation, rows of photoreceptor nuclei can be counted reliably. It also allows the investigation of OS arrangements (orderly or disarrayed), and an approximation of inner and outer segment length.

Frozen sections. Frozen sections were performed as described previously [9. Rohrer, B., et al., Role of neurotrophin receptor TrkB in the maturation of rod photoreceptors and establishment of synaptic transmission to the inner retina. J. Neurosci., 1999. 19(20): p. 8919-8930]. Tissue was fixed in 4% paraformaldehyde and sectioned using a cryostat. After the slides were washed in PBS, and stained with toluidine blue solution (1% tol blue, 1% borax in dH₂O) there were coverslipped using aqueous mounting media.

Cell Counts.

Photoreceptor layers were counted as described previously, [2. Rohrer, B., et al., Lack of p75 receptor does not protect photoreceptors from light-induced cell death. Exp Eye Res, 2003. 76(1): p. 125-9] counting in two locations in the central retina (superior and inferior, within 350 m of the optic nerve head) and two in the peripheral retina (superior and inferior, within 350 μm of the ciliary body). At each location 3 measurements were obtained, which were averaged to provide a single value for each area.

### H. REFERENCES

Abbott, B. J., Fukuda, D. S., Dorman, D. E., Occolowitz, J. L., Debono, M., and Farhner, L. (1979) Antimicrob. Agents Chemother 16(6), 808-812
Ablonczy Z, Crouch R K, Goletz P W, Redmond T M, Knapp D R, Ma J-x, Rohrer B: 11-cis retinal reduces constitutive opsin phosphorylation and improves quantum catch in retinoid deficient mouse rod photoreceptors. J Biol Chem 2002, 277(43):40491-40498.
Acosta M L, Fletcher E L, Azizoglu S, Foster L E, Farber D B, Kalloniatis M: Early markers of retinal degeneration in rd/rd mice. Mol Vis 2005, 11:717-728.
Bayer, A. U., et al., Evaluation of different recording parameters to establish a standard for flash electroretinography in rodents. Vis. Res., 2001. 41(17): p. 2173-2185.
Cingolani C, Rogers B, Lu L, Kachi S, Shen J, Campochiaro P A: Retinal degeneration from oxidative damage. Free Radic Biol Med 2006, 40(4):660-669.
Cochemé, H. M., and Murphy, M. P. (2008) J. Biol. Chem. 283(4), 1786-1798 Eklund S E, Taylor D, Kozlov E, Prokop A, Cliffel D E: A microphysiometer for simultaneous measurement of changes in extracellular glucose, lactate, oxygen, and acidification rate. Anal Chem 2004, 76(3):519-527.
Faktorovich, E. G., et al., Basic fibroblast growth factor and local injury protect photoreceptors from light damage in the rat. J. Neurosci., 1992. 12(9): p. 3554-3567.
Farber D B, Lolley R N: Cyclic guanosine monophosphate: elevation in degenerating photoreceptor cells of the C3H mouse retina. Science 1974, 186:449-451.
Farber D B: From mice to men: the cyclic GMP phosphodiesterase gene in vision and disease. The Proctor Lecture. Invest Ophthalmol Vis Sci 1995, 36(2):263-275.
Fox D A, Poblenz A T, He L: Calcium overload triggers rod photoreceptor apoptotic cell death in chemical-induced and inherited retinal degenerations. Ann NY Acad Sci 1999, 893:282-285.
Fukushima, T., Tanaka, K., Lim, H., and Moriyama, M. (2002) Environ. Health Prevent. Med. 7, 89-94
Graymore C: Metabolism of the Developing Retina. 7. Lactic Dehydrogenase Isoenzyme in the Normal and Degenerating Retina. a Preliminary Communication. Exp Eye Res 1964, 89:5-8.
Gresh J, Goletz P W, Crouch R K, Rohrer B: Structure-function analysis of rods and cones in juvenile, adult, and aged C57bl/6 and Balb/c mice. Vis Neurosci 2003, 20(2):211-220.
Hajnóczky, G., Davies, E., and Madesh, M. (2003) Biochem. Biophys. Res. Commun. 304, 445-454
Hassan, H. M. (1984) Methods Enzymol. 105, 523-532
Houston J G, Banks M N, Binnie A, Brenner S, O'Connell J, Petrillo E W: Case study: impact of technology investment on lead discovery at Bristol-Myers Squibb, 1998-2006. Drug Discov Today 2008, 13(1-2):44-51.
Jeon C J, Strettoi E, Masland R H: The major cell populations of the mouse retina. J Neurosci 1998, 18(21):8936-8946.
Koutalos, Y., and Yau, K.-W. (1996) Trends Neurosci. 19, 73-81
Kunchithapautham K, Rohrer B: Apoptosis and Autophagy in Photoreceptors Exposed to Oxidative Stress. Autophagy 2007, 3(5).
Lenz E M, Wilson I D: Analytical strategies in metabonomics. J Proteome Res 2007, 6(2):443-458.
Li, Z. Y., et al., Amelioration of photic injury in rat retina by ascorbic acid: a histopathologic study. Invest Ophthalmol Vis Sci, 1985. 26(11): p. 1589-98.
Lipton, S. A., Rasmussen, H., and Dowling, J. E. (1977) J. Gen. Physiol. 70, 771-791
Lohr H R, Kuntchithapautham K, Sharma A K, Rohrer B: Multiple, parallel cellular suicide mechanisms participate in photoreceptor cell death. Exp Eye Res 2006, 83(2):380-389.
Lyubarsky, A. L. and E. N. Pugh Jr., Recovery phase of the murine rod photoresponse reconstructed from electroretinographic recordings. J. Neurosci., 1996. 16(2): p. 563-571.
Mattson M P, Kroemer G: Mitochondria in cell death: novel targets for neuroprotection and cardioprotection. Trends Mol Med 2003, 9(5):196-205.
Medrano C J, Fox D A. (1995) Exp. Eye Res. 61(3):273-84.
Medrano C J, Fox D A: Oxygen consumption in the rat outer and inner retina: light- and pharmacologically-induced inhibition. Exp Eye Res 1995, 61(3):273-284.
Moriguchi, K., et al., Suppression of N-methyl-N-nitrosourea-induced photoreceptor apoptosis in rats by docosahexaenoic acid. Ophthalmic Res, 2004. 36(2): p. 98-105.
Nicholas P C, Kim D, Crews F T, Macdonald J M: (1)H NMR-Based Metabolomic Analysis of Liver, Serum, and Brain Following Ethanol Administration in Rats. Chem Res Toxicol 2007.
Noell, W. K., et al., Ascorbate and dietary protective mechanisms in retinal light damage of rats: electrophysiological, histological and DNA measurements. Prog Clin Biol Res, 1987. 247: p. 469-83.
Ogilvie, J. M., et al., A reliable method for organ culture of neonatal mouse retina with long-term survival. J. Neurosci. Methods, 1999. 87(1): p. 57-65.
Ohira, A., et al., Glutathione peroxidase induced in rat retinas to counteract photic injury. Invest Ophthalmol Vis Sci, 2003. 44(3): p. 1230-6.
Orrenius, S., Zhivotovsky, B., and Nicotera, P. (2003) Nat. Rev. Mol. Cell. Biol. 4, 552-565
Penn, J. S. and D. H. Anderson, Effects of light history on the rat retina. Progress in Retinal Research, ed. C. G. Osborne N N. 1992, N Y: Pergamon Press. 75-98.
Perlman Z E, Slack M D, Feng Y, Mitchison T J, Wu L F, Altschuler S J: Multidimensional drug profiling by automated microscopy. Science 2004, 306(5699):1194-1198.
Pierce E A, Quinn T, Meehan T, McGee T L, Berson E L, Dryja T P: Mutations in a gene encoding a new oxygen-regulated photoreceptor protein cause dominant retinitis pigmentosa. Nat Genet 1999, 22(3):248-254.
Pierce E A: Pathways to photoreceptor cell death in inherited retinal degenerations. Bioessays 2001, 23(7):605-618.
Pinzon-Duarte, G., et al., Cell differentiation, synaptogenesis, and influence of the retinal pigment epithelium in a rat neonatal organotypic retina culture. Vision Res, 2000. 40(25): p. 3455-65.
Richards A, Emondi A A, Rohrer B: Long-term ERG analysis in the partially light-damaged mouse retina reveals regressive and compensatory changes. Vis Neurosci 2006, 23(1):91-97.
Rohrer B, Blanco R, Marc R E, Lloyd M B, Bok D, Schneeweis D M, Reichardt L F: Functionally intact glutamate-mediated signaling in bipolar cells of the TRKB knockout mouse retina. Vis Neurosci 2004, 21(5):703-713.
Rohrer B, Korenbrot J I, LaVail M M, Reichardt L F, Xu B: Role of neurotrophin receptor TrkB in the maturation of rod photoreceptors and establishment of synaptic transmission to the inner retina. J Neurosci 1999, 19(20):8919-8930.
Rohrer B, Matthes M T, LaVail M M, Reichardt L F: Lack of p75 receptor does not protect photoreceptors from light-induced cell death. Exp Eye Res 2003, 76(1):125-129.
Rohrer, B. and J. M. Ogilvie, Retarded outer segment development in TrkB knockout mouse retina organ culture. Mol Vis, 2003. 9: p. 18-23.
Sanvicens N, Gomez-Vicente V, Masip I, Messeguer A, Cotter T G: Oxidative stress-induced apoptosis in retinal photoreceptor cells is mediated by calpains and caspases and blocked by the oxygen radical scavenger CR-6. J Biol Chem 2004, 279(38):39268-39278.
Schnellmann R G: Measurement of oxygen consumption, vol. 1 (Part B) In vitro toxicity indicators; 1998.
Sharma A K, Rohrer B: Calcium-induced calpain mediates apoptosis via caspase-3 in a mouse photoreceptor cell line. J Biol Chem 2004, 279(34):35564-35572.
Sharma A K, Rohrer B: Sustained elevation of intracellular cGMP causes oxidative stress triggering calpain-mediated apoptosis in photoreceptor degeneration. Curr Eye Res 2007, 32(3):259-269.
Specht, S., et al., Damage to rat retinal DNA induced in vivo by visible light. Photochem Photobiol, 1999. 69(1): p. 91-8.
Stone J, Maslim J, Valter-Kocsi K, Mervin K, Bowers F, Chu Y, Barnett N, Provis J, Lewis G, Fisher S K et al: Mechanisms of photoreceptor death and survival in mammalian retina. Prog Retin Eye Res 1999, 18(6):689-735.
Tan E, Ding X Q, Saadi A, Agarwal N, Naash M I, Al-Ubaidi M R: Expression of cone-photoreceptor-specific antigens in a cell line derived from retinal tumors in transgenic mice. Invest Ophthalmol Vis Sci 2004, 45(3):764-768.
Tanito, M., et al., Attenuation of retinal photooxidative damage in thioredoxin transgenic mice. Neurosci Lett, 2002. 326(2): p. 142-6.
Travis G H: Mechanisms of cell death in the inherited retinal degenerations. Am J Hum Genet 1998, 62(3):503-508.
Usachev, Y., and Verkhratsky, A. (1995) Cell Calcium 17(3), 197-206
Vingolo E M, De Mattia G, Giusti C, Forte R, Laurenti O, Pannarale M R: Treatment of nonproliferative diabetic retinopathy with Defibrotide in noninsulin-dependent diabetes mellitus: a pilot study. Acta Ophthalmol Scand 1999, 77(3):315-320.
Wenzel A, Grimm C, Samardzija M, Reme C E: Molecular mechanisms of light-induced photoreceptor apoptosis and neuroprotection for retinal degeneration. Prog Retin Eye Res 2005, 24(2):275-306.
Wiley C, Beeson C: Continuous measurement of glucose utilization in heart myoblasts. Anal Biochem 2002, 304(2):139-146.
Winkler B S, Pourcho R G, Starnes C, Slocum J, Slocum N: Metabolic mapping in mammalian retina: a biochemical and 3H-2-deoxyglucose autoradiographic study. Exp Eye Res 2003, 77(3):327-337.
Winkler B S, Starnes C A, Sauer M W, Firouzgan Z, Chen S C: Cultured retinal neuronal cells and Muller cells both show net production of lactate. Neurochem Int 2004, 45(2-3):311-320.
Yarfitz, S., and Hurley, J. B. (1994) J. Biol. Chem. 269(20), 14329-14332
Zhang, X., Feng, Q., and Cote, R. H. (2005) Invest. Ophthalmol. Vis. Sci. 46(9), 3060-3066

1-70. (canceled)

71. A compound of formula (II): 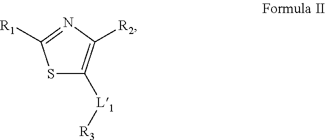
wherein:
R₁ is phenyl or pyridyl;
R₂ is phenyl or pyridyl;
R₃ is phenyl or pyridyl;
L′₁ is selected from the group consisting of alkyl, amine and amide, said L′₁ optionally substituted with one or more substituents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, and —S(O)₂NR¹⁰¹R¹⁰²;
R¹⁰¹ and R¹⁰² are each independently selected from the group consisting of hydrogen, C₃ to C₂₀ alkyl, alkenyl, alkynyl, cycloalkyl, aryl, heterocycloalkyl and heteroaryl; and
wherein each R¹⁰¹ and R¹⁰² are independently unsubstituted or substituted with one or more substituents independently selected from the group consisting of halogen; hydroxyl; cyano; nitro; amino; alkylamino; dialkylamino; alkyl unsubstituted or substituted with one or more halogen or alkoxy or aryloxy; aryl unsubstituted or substituted with one or more halogen or alkoxy or alkyl or trihaloalkyl; heterocycloalkyl unsubstituted or substituted with aryl or heteroaryl or ═O or alkyl unsubstituted or substituted with hydroxyl; cycloalkyl unsubstituted or substituted with hydroxyl; heteroaryl unsubstituted or substituted with one or more halogen or alkoxy or alkyl or trihaloalkyl; haloalkyl; hydroxyalkyl; carboxy; alkoxy; aryloxy; alkoxycarbonyl; aminocarbonyl; alkylaminocarbonyl and dialkylaminocarbonyl,
or a pharmaceutically acceptable salt thereof.

72. The compound according to claim 71, wherein R₁ is pyridyl.

73. The compound according to claim 71, wherein R₂ is phenyl.

74. The compound according to claim 71, wherein R₃ is phenyl.

75. The compound according to claim 71, wherein L′₁ is —CH₂NH—, —CH₂CH₂—, —C(O)NH—, —CH(OH)CH₂—, —C═C—, —C═N— or —CH₂O—.

76. The compound according to claim 71, wherein said compound is: 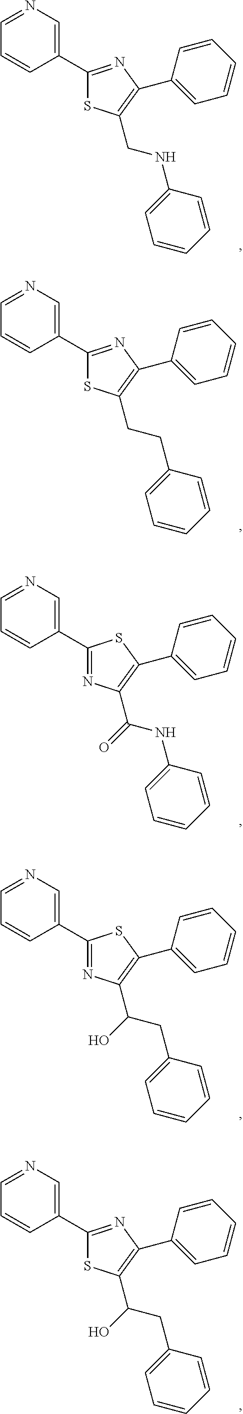 [SMILES: c1ccc(NCc2sc(-c3cccnc3)nc2-c2ccccc2)cc1 ; c1ccc(CCc2sc(-c3cccnc3)nc2-c2ccccc2)cc1 ; O=C(Nc1ccccc1)c1nc(-c2cccnc2)sc1-c1ccccc1 ; OC(Cc1ccccc1)c1nc(-c2cccnc2)sc1-c1ccccc1 ; OC(Cc1ccccc1)c1sc(-c2cccnc2)nc1-c1ccccc1] 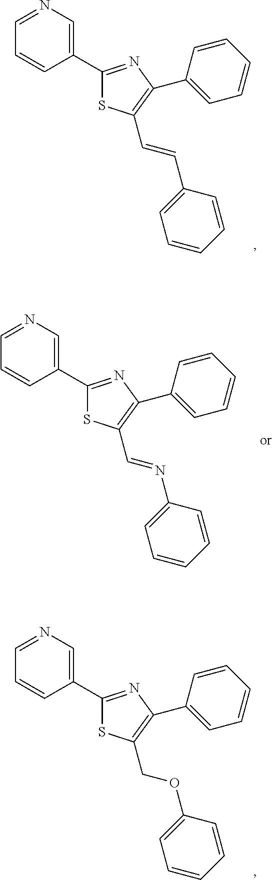 [SMILES: C(=C\c1sc(-c2cccnc2)nc1-c1ccccc1)/c1ccccc1 ; C(=N\c1ccccc1)/c1sc(-c2cccnc2)nc1-c1ccccc1 ; c1ccc(OCc2sc(-c3cccnc3)nc2-c2ccccc2)cc1]
or a pharmaceutically acceptable salt thereof.

77. A pharmaceutical composition, comprising a therapeutically effective amount of a compound according to claim 71 and a pharmaceutically acceptable excipient.
